(19) United States
(12) Patent Application Publication
(10) Pub. No.: US 2025/0325388 A1
(43) Pub. Date: Oct. 23, 2025

(54) SEALING CAVAL DOCKING IMPLANTS

(71) Applicant: EDWARDS LIFESCIENCES CORPORATION, Irvine, CA (US)

(72) Inventors: Michael G. Valdez, Riverside, CA (US); Omar Fawzi Azanki, Coto de Caza, CA (US); David Lyle Leibowitz, Capistrano Beach, CA (US); Anne Bernadette Aragon Alcasid, Costa Mesa, CA (US); Tiana (formerly Thu Thi Anh) Tran, Westminster, CA (US); John Charles Laschinger, Laguna Beach, CA (US); Leonardo Paim Nicolau Da Costa, Tampa, FL (US); Meredith Gage, Newport Beach, CA (US); Sean Timothy O'keeffe, Tustin, CA (US); Robin Wooding Eckert, Irvine, CA (US)

(73) Assignee: EDWARDS LIFESCIENCES CORPORATION, Irvine, CA (US)

(21) Appl. No.: 19/252,125

(22) Filed: Jun. 27, 2025

Related U.S. Application Data
(63) Continuation of application No. PCT/US2024/010728, filed on Jan. 8, 2024.
(60) Provisional application No. 63/479,270, filed on Jan. 10, 2023.
(60) Provisional application No. 63/488,515, filed on Mar. 5, 2023.

(51) Int. Cl. A61F 2/856

(52) U.S. Cl. CPC A61F 2/856; A61F 2230/0006; A61F 2230/0091; A61F 2250/0003; A61F 2250/0036; A61F 2250/0069

(57) ABSTRACT

A cardiac implant device comprises a frame configured for placement at least partially within a blood vessel of a heart, a skirt configured to at least partially enclose the frame, and two or more arms extending from a distal end of the frame.

### RELATED APPLICATION

This application is a continuation of PCT Patent Application No. PCT/US2024/010728 filed on Jan. 8, 2024, which application claims priority to U.S. Provisional Application No. 63/479,270, filed Jan. 10, 2023, and entitled SEALING CAVAL DOCKING IMPLANTS and to U.S. Provisional Application No. 63/488,515, filed Mar. 5, 2023, and entitled CAVAL VALVE WITH DISTAL ARMS, the disclosures of which are hereby incorporated by reference in their entirety.

### BACKGROUND

### Field

The present disclosure generally relates to the field of a medical implant devices.

### Description of Related Art

Various medical procedures involve the implantation of medical implant devices within the anatomy of the heart. Certain physiological parameters associated with such anatomy, such as fluid pressure, can have an impact on patient health prospects.

### SUMMARY

Described herein are one or more methods and/or devices to facilitate monitoring of physiological parameter(s) associated with certain chambers and/or vessels of the heart, such as the left atrium, using one or more sensor implant devices.

Some implementations of the present disclosure relate to a cardiac implant device including: a frame configured for placement at least partially within a blood vessel of a heart; a skirt configured to at least partially enclose the frame; and two or more arms extending from a distal end of the frame.

In some aspects, the techniques described herein relate to a cardiac implant device, wherein the two or more arms are at least partially curved.

In some aspects, the techniques described herein relate to a cardiac implant device, wherein the two or more arms have a wavy form.

In some aspects, the techniques described herein relate to a cardiac implant device or claim 3, wherein the two or more arms form a bulb.

In some aspects, the techniques described herein relate to a cardiac implant device, wherein the two or more arms are configured to straighten in response to blood pressure through the frame.

In some aspects, the techniques described herein relate to a cardiac implant device, wherein the skirt is further configured to at least partially enclose the two or more arms.

In some aspects, the techniques described herein relate to a cardiac implant device including: a frame configured for placement at least partially within a blood vessel of a heart; and a skirt configured to improve a seal of the frame within the blood vessel.

In some aspects, the techniques described herein relate to a cardiac implant device, wherein the skirt includes a relatively thin portion and a relatively thick portion.

In some aspects, the techniques described herein relate to a cardiac implant device, wherein the relatively thick portion has a zigzag form.

In some aspects, the techniques described herein relate to a cardiac implant device or claim 10, wherein the relatively thick portion forms an elongate column extending longitudinally along the frame.

In some aspects, the techniques described herein relate to a cardiac implant device, wherein the skirt includes multiple relatively thin portions and multiple relatively thick portions, and wherein the relatively thin portions and the relatively thick portions are positioned in an alternating manner.

In some aspects, the techniques described herein relate to a cardiac implant device, wherein the relatively thick portion includes an elongate form and extends laterally relative to the frame.

In some aspects, the techniques described herein relate to a cardiac implant device, wherein the relatively thick portion has a half dome shape.

In some aspects, the techniques described herein relate to a cardiac implant device, further including one or more flaps coupled to an exterior surface of the skirt.

In some aspects, the techniques described herein relate to a cardiac implant device, wherein the one or more flaps are arranged in series forming a helical pattern around the skirt.

In some aspects, the techniques described herein relate to a cardiac implant device, wherein the skirt includes a base coupled to a distal end of the frame and further includes a free end.

In some aspects, the techniques described herein relate to a cardiac implant device, wherein the frame includes a lateral extension configured for placement within a branching blood vessel.

In some aspects, the techniques described herein relate to a cardiac implant device, wherein the skirt has a ring shape.

In some aspects, the techniques described herein relate to a cardiac implant device, wherein the skirt includes a rigid portion and a flexible portion.

In some aspects, the techniques described herein relate to a cardiac implant device, wherein the skirt includes one or more apertures disposed at a distal end of the skirt.

For purposes of summarizing the disclosure, certain aspects, advantages, and novel features have been described. It is to be understood that not necessarily all such advantages may be achieved in accordance with any particular example. Thus, the disclosed examples may be carried out in a manner that achieves or optimizes one advantage or group of advantages as taught herein without necessarily achieving other advantages as may be taught or suggested herein.

### BRIEF DESCRIPTION OF THE DRAWINGS

Various examples are depicted in the accompanying drawings for illustrative purposes and should in no way be interpreted as limiting the scope of the inventions. In addition, various features of different disclosed examples can be combined to form additional examples, which are part of this disclosure. Throughout the drawings, reference numbers may be reused to indicate correspondence between reference elements.

FIG. 1 illustrates an example representation of a human heart in accordance with one or more examples.

FIGS. 2A and 2B illustrate an example stent and/or docking device configured for docking one or more prosthetic valves and comprising a distal sealing element at or near a distal end of the stent configured to increase surface friction at the distal end of the stent in accordance with one or more examples.

FIG. 3 provides a side view of an example stent and/or docking device configured for docking one or more prosthetic valves and comprising a distal sealing element at or near a distal end of the stent configured to increase surface friction at the distal end of the stent in accordance with one or more examples.

FIG. 4 provides a top view of an example stent and/or docking device configured for docking one or more prosthetic valves and comprising a distal sealing element 404 at or near a distal end of the stent configured to increase surface friction at the distal end of the stent in accordance with one or more examples.

FIG. 5 provides a side view of an example stent and/or docking device configured for docking one or more prosthetic valves and comprising a distal sealing element at or near a distal end of the stent configured to increase surface friction at the distal end of the stent in accordance with one or more examples.

FIG. 6 provides a side view of an example stent and/or docking device configured for docking one or more prosthetic valves and comprising a distal sealing element at or near a distal end of the stent configured to increase surface friction at the distal end 607 of the stent in accordance with one or more examples.

FIG. 7 provides a side view of an example stent and/or docking device configured for docking one or more prosthetic valves and comprising a distal sealing element at or near a distal end of the stent configured to increase surface friction at the distal end of the stent in accordance with one or more examples.

FIGS. 8A and 8B illustrate an example stent and/or docking devices (e.g., docking stents and/or docking stations) configured for docking one or more prosthetic valves in accordance with one or more examples.

FIGS. 9A and 9B illustrate an example stent and/or docking devices (e.g., docking stents and/or docking stations) configured for docking one or more prosthetic valves in accordance with one or more examples.

FIG. 10 illustrates an example stent and/or docking device (e.g., docking stent and/or docking station) configured for docking one or more prosthetic valves in accordance with one or more examples.

FIGS. 11A-11C illustrate an example stent and/or docking device (e.g., docking stent and/or docking station) configured for docking one or more prosthetic valves in accordance with one or more examples.

FIGS. 12A-12D illustrate components of an example sealing element for use with various stents and/or docking stations, including those described herein, in accordance with one or more examples.

FIG. 13 illustrates an example stent comprising a stent body (e.g., frame) and a sealing element, in accordance with one or more examples.

FIGS. 14A and 14B illustrate another example stent and/or docking station configured for placement withing one or more blood vessels and/or for docking one or more prosthetic valves in accordance with one or more examples.

FIGS. 15A and 15B illustrate an example stent and/or docking device configured to be implanted at various blood vessels, valves, and/or other anatomies of a heart in accordance with one or more examples.

FIG. 16 illustrates another example stent and/or docking device configured to be implanted at various blood vessels, valves, and/or other anatomies of a heart in accordance with one or more examples.

FIG. 17 illustrates another example stent and/or docking device configured to be implanted at various blood vessels, valves, and/or other anatomies of a heart in accordance with one or more examples.

FIG. 18 illustrates an example flat pattern of an example frame of a stent and/or docking device in accordance with one or more examples.

FIG. 19 illustrates an example stent and/or docking device configured for docking one or more prosthetic valves and comprising a distal sealing element at or near a distal end of the stent configured to increase surface friction at the distal end of the stent in accordance with one or more examples.

FIG. 20 illustrates a sealing element and/or a frame of a sealing element for use with various stents and/or implants described herein and/or otherwise.

FIGS. 21A and 21B illustrate an implantable device with tissue covered and/or inwardly-biased distal arms configured to allow blood to flow in at least one direction and/or in only one direction in accordance with one or more examples.

FIGS. 22A and 22B illustrate an implantable device with tissue covered and/or inwardly-biased distal arms configured to allow blood to flow in at least one direction and/or in only one direction in accordance with one or more examples.

FIG. 23 illustrates an example device implanted at a junction between an inferior vena cava and a right atrium of a heart in accordance with one or more examples.

FIG. 24 provides a flowchart illustrating an example process for delivering various blood flow devices described herein, including the devices described in FIGS. 21-23.

FIG. 25 illustrates an example delivery system for delivering an implant (e.g., stent and/or docking station) configured to form a seal with surrounding anatomy, in accordance with one or more examples.

FIGS. 26A-26B illustrate an example implant comprising an interior balloon configured to form and/or facilitate a seal between a frame of the implant and the surrounding anatomy, in accordance with one or more examples.

FIG. 26B illustrates the implant with the balloon in an inflated state.

FIGS. 27A-27B illustrate an example implant comprising an exterior balloon configured to form a seal between a frame of the implant and the surrounding anatomy.

FIGS. 28A-28B illustrate an example implant configured to mitigate and/or prevent backflow (e.g., from the right atrium into the IVC) in accordance with one or more examples.

FIG. 29 illustrates an example frame configured to support and/or provide a skeleton for one or more covers in accordance with one or more examples.

FIG. 30 illustrates an example docking system for docking one or more prosthetic valves in accordance with one or more examples.

FIG. 31 illustrates an example docking stent configured for docking one or more prosthetic valves in accordance with one or more examples.

FIG. 32 illustrates an example docking stent comprising one or more proximal arms and/or distal arms and configured for docking one or more prosthetic valves in accordance with one or more examples.

FIG. 33 illustrates an example docking stent comprising one or more proximal arms and/or distal arms deployed at an RA/IVC junction in accordance with one or more examples.

FIGS. 34A and 34B illustrate an example docking stent comprising an adjustable sealing skirt in accordance with one or more examples.

FIG. 35 illustrates a docking stent comprising a sealing portion configured to close gaps between the stent and the surrounding tissue, in accordance with one or more examples.

FIG. 36 illustrates an example sealing portion comprising terry-cloth and/or other material comprising loops and/or fibers, in accordance with one or more examples.

FIG. 37 illustrates an example sealing portion comprising tufts added to the sealing portion material using multifilament thread or sutures, in accordance with one or more examples.

FIG. 38 illustrates an example sealing portion comprising a polymer coating configured to react to blood to expand and/or fill gaps between a stent and surrounding tissue, in accordance with one or more examples.

FIG. 39 illustrates a docking stent comprising a sealing portion configured to close gaps between the stent and the surrounding tissue, in accordance with one or more examples.

### DETAILED DESCRIPTION

The headings provided herein are for convenience only and do not necessarily affect the scope or meaning of the claimed invention.

Although certain preferred examples and examples are disclosed below, inventive subject matter extends beyond the specifically disclosed examples to other alternative examples and/or uses and to modifications and equivalents thereof. Thus, the scope of the claims that may arise herefrom is not limited by any of the particular examples described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain examples; however, the order of description should not be construed to imply that these operations are order dependent. Additionally, the structures, systems, and/or devices described herein may be embodied as integrated components or as separate components. For purposes of comparing various examples, certain aspects and advantages of these examples are described. Not necessarily all such aspects or advantages are achieved by any particular example. Thus, for example, various examples may be carried out in a manner that achieves or optimizes one advantage or group of advantages as taught herein without necessarily achieving other aspects or advantages as may also be taught or suggested herein.

Certain reference numbers are re-used across different figures of the figure set of the present disclosure as a matter of convenience for devices, components, systems, features, and/or modules having features that may be similar in one or more respects. However, with respect to any of the examples disclosed herein, re-use of common reference numbers in the drawings does not necessarily indicate that such features, devices, components, or modules are identical or similar. Rather, one having ordinary skill in the art may be informed by context with respect to the degree to which usage of common reference numbers can imply similarity between referenced subject matter. Use of a particular reference number in the context of the description of a particular figure can be understood to relate to the identified device, component, aspect, feature, module, or system in that particular figure, and not necessarily to any devices, components, aspects, features, modules, or systems identified by the same reference number in another figure. Furthermore, aspects of separate figures identified with common reference numbers can be interpreted to share characteristics or to be entirely independent of one another.

Certain standard anatomical terms of location are used herein to refer to the anatomy of animals, and namely humans, with respect to the preferred examples. Although certain spatially relative terms, such as “outer,” “inner,” “upper,” “lower,” “below,” “above,” “vertical,” “horizontal,” “top,” “bottom,” and similar terms, are used herein to describe a spatial relationship of one device/element or anatomical structure to another device/element or anatomical structure, it is understood that these terms are used herein for case of description to describe the positional relationship between element(s)/structures(s), as illustrated in the drawings. It should be understood that spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa.

The present disclosure relates to systems, devices, and methods for delivery of prosthetic heart valves and/or for managing blood flow within a heart. In some implementations, the present disclosure relates to cardiac implant devices that incorporate or are associated with prosthetic valves. The term “associated with” is used herein according to its broad and ordinary meaning. For example, where a first feature, element, component, device, or member is described as being “associated with” a second feature, element, component, device, or member, such description should be understood as indicating that the first feature, element, component, device, or member is physically coupled, attached, or connected to, integrated with, embedded at least partially within, or otherwise physically related to the second feature, element, component, device, or member, whether directly or indirectly. Certain examples are disclosed herein in the context of cardiac implant devices. However, although certain principles disclosed herein are particularly applicable to the anatomy of the heart, it should be understood that sensor implant devices in accordance with the present disclosure may be implanted in, or configured for implantation in, any suitable or desirable anatomy.

In some examples, an implant device may comprise one or more markers, including radiopaque markers, to facilitate visualization and/or location of the sensor device within a patient's body. A marker may be embedded within a frame and/or sealing element of the implant of the device.

Some implementations of the present disclosure relate to stents and/or similar devices configured for managing blood flow through one or more blood vessels. Some examples relate to stents and/or similar devices comprising prosthetic valves and/or prosthetic valve components (e.g., leaflets) and/or configured for docking a prosthetic valve.

Some examples comprise distal flared ends and/or compressible flanges disposed at or near distal ends of frames of example stent devices. In some examples, a portion of an implant (e.g., a flange and/or distal end) may be configured to extend out of a blood vessel and/or at least partially along a floor of a chamber (e.g., right atrium) of the heart. Some components of example implants may be at least partially enclosed and/or covered by a covering comprising one or more cloths, fabrics, and/or other materials. In some examples, a covering may be configured to increase frictional contact and/or increase friction between an implant and the native anatomy.

Some example implants may comprise various features and/or components configured to minimize contact and/or blockage of one or more branching blood vessels (e.g., hepatic veins). For example, one or more implants may comprise inwardly bent walls and/or inward bulges at or near a midsection of the implant such that the implant recedes from the walls of a main blood vessel and/or from an inflow junction of one or more branching blood vessels. In some examples, an implant may comprise a network of wires and/or struts configured to form cells having any suitable shapes and/or sizes. In some examples, one or more cells at or near a midsection of the implant may be larger than cells at other portions of the implant.

While some implants described herein may be configured to receive and/or dock one or more prosthetic valves, some example implants may comprise incorporated valves and/or valve components (e.g., leaflets). For example, any of the implants described herein may comprise coupled and/or affixed leaflets configured to extend across lumens of the implants (e.g., at distal ends of the implants). In some examples, leaflets may extend from a frame of the implant. The frame may have a compressible, expandible, and/or flexible structure.

Example implants can comprise various features for anchoring to the native anatomy. In some examples, an implant may comprise one or more inward and/or outward extending arms. Some example anchoring arms may extend longitudinally and/or laterally from a frame of the implant. In some examples, one or more anchoring arms may comprise various anchoring features, which can include barbed ends and/or similar features.

The present disclosure relates to implants in one or more blood vessels, including inferior vena cava and/or superior vena cava. Some example implants may comprise laser-cut nitinol tube frames and/or a sealing skirt attached at one end (e.g., at a distal end configured to extend into the right atrium and/or other chamber). Some implants may be relatively long and/or may comprise a sealing skirt that extends a sufficient distance such that, for some patients, the sealing skirt can at least partially occlude one or more branching blood vessels (e.g., the hepatic veins). Because of the variation in exactly where the hepatic veins and/or other branching blood vessels enter the vena cava and/or other blood vessels, some implants described herein may be applicable for variably sized and/or shaped anatomies.

### Cardiac Physiology

The anatomy of the heart is described below to assist in the understanding of certain inventive concepts disclosed herein. In humans and other vertebrate animals, the heart generally comprises a muscular organ having four pumping chambers, wherein the flow thereof is at least partially controlled by various heart valves, namely, the aortic, mitral (or bicuspid), tricuspid, and pulmonary valves. The valves may be configured to open and close in response to a pressure gradient present during various stages of the cardiac cycle (e.g., relaxation and contraction) to at least partially control the flow of blood to a respective region of the heart and/or to blood vessels (e.g., pulmonary, aorta, etc.).

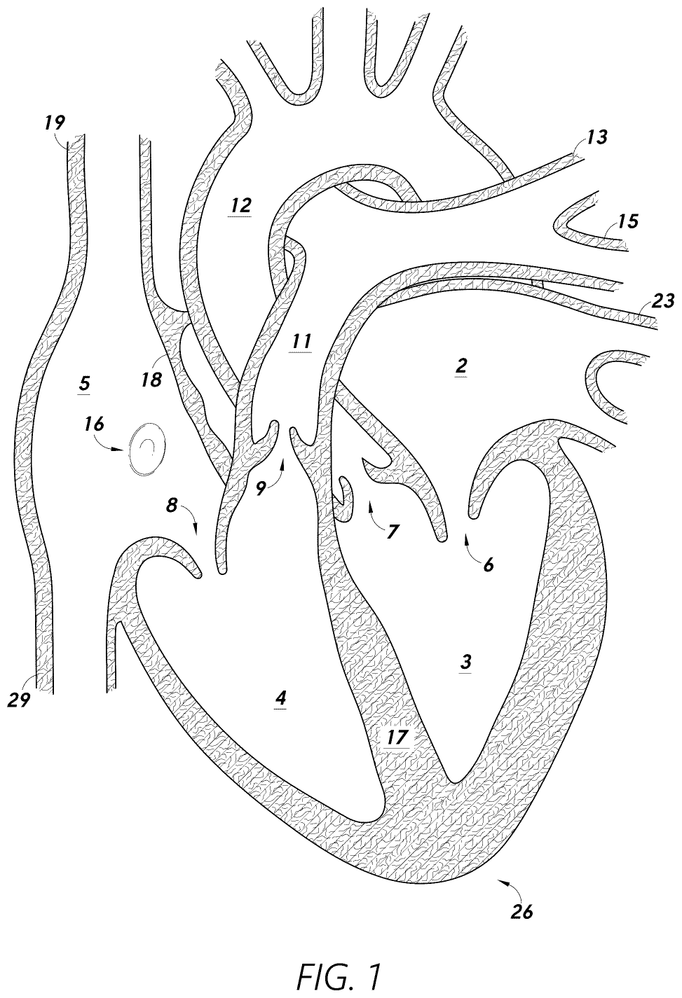

FIG. 1 illustrates an example representation of a heart 1 having various features relevant to certain examples of the present inventive disclosure. The heart 1 includes four chambers, namely the left atrium 2, the left ventricle 3, the right ventricle 4, and the right atrium 5. In terms of blood flow, blood generally flows from the right ventricle 4 into the pulmonary artery 11 via the pulmonary valve 9, which separates the right ventricle 4 from the pulmonary artery 11 and is configured to open during systole so that blood may be pumped toward the lungs and close during diastole to prevent blood from leaking back into the heart from the pulmonary artery 11. The pulmonary artery 11 carries deoxygenated blood from the right side of the heart to the lungs. The pulmonary artery 11 includes a pulmonary trunk and left 15 and right 13 pulmonary arteries that branch off of the pulmonary trunk, as shown. The pulmonary veins 23 carry blood from the lungs to the left atrium 2.

In addition to the pulmonary valve 9, the heart 1 includes three additional valves for aiding the circulation of blood therein, including the tricuspid valve 8, the aortic valve 7, and the mitral valve 6. The tricuspid valve 8 separates the right atrium 5 from the right ventricle 4. The tricuspid valve 8 generally has three cusps or leaflets and may generally close during ventricular contraction (i.e., systole) and open during ventricular expansion (i.e., diastole). The mitral valve 6 generally has two cusps/leaflets and separates the left atrium 2 from the left ventricle 3. The mitral valve 6 is configured to open during diastole so that blood in the left atrium 2 can flow into the left ventricle 3, and, when functioning properly, closes during systole to prevent blood from leaking back into the left atrium 2. The aortic valve 7 separates the left ventricle 3 from the aorta 12. The aortic valve 7 is configured to open during systole to allow blood leaving the left ventricle 3 to enter the aorta 12, and close during diastole to prevent blood from leaking back into the left ventricle 3.

The heart valves may generally comprise a relatively dense fibrous ring, referred to herein as the annulus, as well as a plurality of leaflets or cusps attached to the annulus. Generally, the size of the leaflets or cusps may be such that when the heart contracts the resulting increased blood pressure produced within the corresponding heart chamber forces the leaflets at least partially open to allow flow from the heart chamber. As the pressure in the heart chamber subsides, the pressure in the subsequent chamber or blood vessel may become dominant and press back against the leaflets. As a result, the leaflets/cusps come in apposition to each other, thereby closing the flow passage. Dysfunction of a heart valve and/or associated leaflets (e.g., pulmonary valve dysfunction) can result in valve leakage and/or other health complications.

The atrioventricular (i.e., mitral and tricuspid) heart valves may further comprise a collection of chordae tendineae and papillary muscles (not shown) for securing the leaflets of the respective valves to promote and/or facilitate proper coaptation of the valve leaflets and prevent prolapse thereof. The papillary muscles, for example, may generally comprise finger-like projections from the ventricle wall. The valve leaflets are connected to the papillary muscles by the chordae tendineae. A wall of muscle, referred to as the septum, separates the left-side chambers from the right-side chambers. In particular, an atrial septum wall portion 18 (referred to herein as the “atrial septum,” “interatrial septum,” or “septum”) separates the left atrium 2 from the right atrium 5, whereas a ventricular septum wall portion 17 (referred to herein as the “ventricular septum,” “interventricular septum,” or “septum”) separates the left ventricle 3 from the right ventricle 4. The inferior tip 26 of the heart 1 is referred to as the apex and is generally located on or near the midclavicular line, in the fifth intercostal space.

The coronary sinus 16 comprises a collection of veins joined together to form a large vessel that collects blood from the heart muscle (myocardium). The ostium of the coronary sinus, which can be guarded at least in part by a Thebesian valve in some patients, is open to the right atrium 5, as shown. The coronary sinus runs along a posterior aspect of the left atrium 2 and delivers less-oxygenated blood to the right atrium 5. The coronary sinus generally runs transversely in the left atrioventricular groove on the posterior side of the heart.

Any of several access pathways in the heart 1 may be utilized for maneuvering guidewires and catheters in and around the heart 1 to deploy implants and/or devices of the present application. For instance, access may be from above via either the subclavian vein or jugular vein into the superior vena cava (SVC) 19, right atrium 5, and from there into the coronary sinus 16. Alternatively, the access path may start in the femoral vein and through the inferior vena cava (IVC) 14 into the heart 1. Other access routes may also be used, and each can utilize a percutaneous incision through which the guidewire and catheter are inserted into the vasculature, normally through a sealed introducer, and from there the physician can control the distal ends of the devices from outside the body.

### Sealing Caval Implant Devices

Heart valves (including transcatheter valves) can be deployed independently and/or using one or more docking devices (i.e., docking stations). Some docking devices can comprise an expandable frame, one or more scaling portions, and/or a valve seat and/or mount for the heart valve. The expandable frame can be configured to conform to an interior shape of a portion of the circulatory system when expanded inside the circulatory system. Some docking devices can be deployed in the inferior vena cava (IVC), superior vena cava (SVC), both the IVC and SVC, and/or other areas of the anatomy.

Heart valves and/or docking devices can be used in various treatments, which can include treating patients with symptomatic heart failure and signs of peripheral and venous congestion resulting from reverse caval flow.

Examples described herein relate to stents and/or docking devices (e.g., docking stents and/or docking stations) configured for docking one or more prosthetic valves. In some examples, a docking device may be configured for deployment at a junction (e.g., inflow junction) of the right atrium (RA) and IVC. However, example devices (e.g., docking devices) and/or stents described herein can be configured for deployment at other areas of the anatomy.

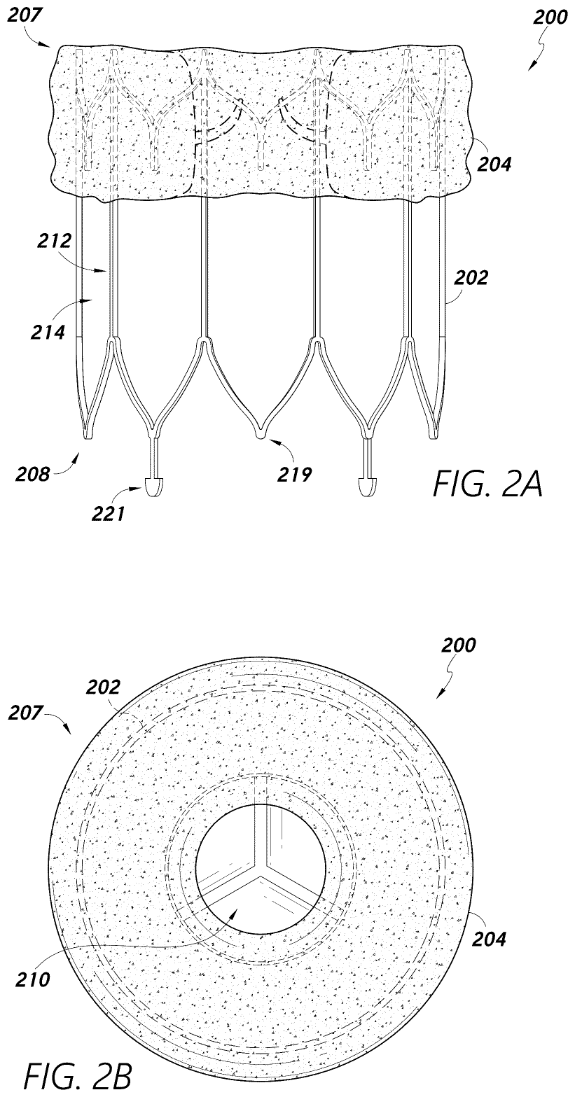

FIGS. 2A and 2B illustrate an example stent 200 and/or docking device configured for docking one or more prosthetic valves and comprising a distal sealing element 204 at or near a distal end 207 of the stent 200 configured to increase surface friction at the distal end 207 of the stent in accordance with one or more examples. FIG. 2A provides a side view of the stent 200 and FIG. 2B provides a top view of the stent 200. In some examples, the sealing element 204 can comprise a thick fabric and/or cloth configured to at least partially enclose the distal end 207. The stent 200 may further comprise a frame 202 having a generally cylindrical and/or tubular form. In some examples, the frame 202 may comprise a network of one or more wires 212 (e.g., struts and/or cords) forming one or more cells 214 between the wires 212.

The sealing element 204 and/or cloth may have a generally thick structure and/or may increase a diameter of the frame 202 and/or may extend from the frame 202. The sealing element 204 may be configured to improve sealing of the stent 200 at the distal end 207. In some examples, the sealing element 204 may have a fibrous and/or fuzzy texture to increase friction between the stent 200 and a blood vessel and/or blood vessel junction (e.g., at or near the RA/IVC junction).

While the stent 200 is shown comprising a sealing element 204 only at the distal end 207 of the stent 200, the stent 200 may comprise multiple sealing elements 204 and/or the sealing element 204 may be configured to extend further along the frame 202. In some examples, the sealing element 204 may be disposed at an outer surface of the frame 202. The sealing element 204 may have a ring and/or generally circular shape and/or may be configured to extend along a full circumference of the frame 202.

As shown in FIG. 2B, the sealing element 204 may be configured to extend at least partially over a leading end of the frame 202 and/or to extend onto an inner surface of the frame 202. In some examples, the sealing element 204 may be configured to increase friction and/or grip with a prosthetic valve placed into an inner lumen 210 of the frame 202. For example, the sealing element 204 may increase a width of the stent 200 and/or may be configured to extend from the inner surface of the frame 202 to provide a contact point for one or more prosthetic valves placed into the frame 202. The sealing element 204 may be configured to pinch the prosthetic valve to securely hold the prosthetic valve.

The stent 200 may be configured for delivery and/or placement at an SVC and/or IVC of a heart. For example, the stent 200 may be configured for placement at or near an in-flow junction of the SVC and/or IVC to the right atrium of the heart. The stent 200 may be configured for antegrade delivery (e.g., via the femoral vein) and/or retrograde delivery (e.g., via the subclavian and/or jugular vein).

The frame 202 may be at least partially composed of one or more shape memory alloys (e.g., Nitinol) and/or may be laser-cut into a desired form. In some examples, the frame 202 and/or sealing element 204 may have a generally flared, mushroom, and/or funnel shape and/or form at or near the distal end 207 of the stent 200. For example, the distal end 207 of the stent 200 may be flared and/or conical in shape to extend outward into the right atrium and/or to form a grip onto a rim of the right atrium. In some examples, the sealing element 204 may be configured to extend beyond the frame 202 and/or may form an extension of the frame 202 to extend further laterally and/or longitudinally relative to the frame 202. The frame 202 may further comprise a proximal end 208 having a straight or flared form.

The stent 200 may be configured to present minimal blockage of one or more hepatic veins and/or other branching blood vessels. For example, a heart may generally include one or more hepatic veins branching into the IVC at or near a junction between the IVC and the right atrium. In some examples, the sealing element 204 may not extend along a complete length of the frame 202. For example, the sealing element 204 may extend from the distal end 207 of the stent 200 to a midsection and/or midpoint of the stent 200. The stent 200 may be configured such that, when implanted at the junction between the IVC and the right atrium, the sealing element 204 may not extend over the one or more hepatic veins and/or branching blood vessels. In some examples, the frame 202 may at least partially extend over the hepatic veins and/or branching blood vessels and/or may allow blood flow from the branching blood vessels through cells 214 of the frame 202.

In some examples, the stent 200 may comprise one or more radiopaque markers configured to facilitate delivery and/or use of the stent 200. For example, one or more markers may be disposed along the sealing element 204 to assist with aligning and/or docking one or more prosthetic valves at the stent 200 and/or sealing element 204. In another example, the stent 200 may comprise one or more markers at the frame 202 and/or sealing element 204 at or near the distal end 207 to facilitate positioning of the stent 200 and/or distal end 207 at the atrium junction and/or other anatomy.

In some examples, the stent 200 may be configured to cause native anatomy to conform to a size and/or shape of the stent 200. For example, the frame 202, sealing element 204, and/or distal end 207 of the stent 200 may be configured to press outwardly against the native tissue to create a larger opening to support a prosthetic valve. In some examples, the stent 200 may comprise one or more bulbs configured to press outwardly against walls of a blood vessel and/or may be configured to correspondingly press walls of the blood vessel outwardly. The term “bulb” is used herein in accordance with its plain and ordinary meaning and may refer to any outward- and/or inward-extending portion of a frame 202. In some examples, the frame 202 may increase in diameter at the bulb and/or may decrease in diameter on either side of the bulb.

The frame 202 may be coupled to the sealing element 204 and/or may be configured to mate with the sealing element 204. In some examples, one or more sutures and/or other attachment features (e.g., clips, hooks, notches, stitches, adhesives, etc.) may be used to form an attachment between the frame 202 and the sealing element 204.

The stent 200 may comprise various components and/or features at the proximal end 208 of the frame 202. For example, the frame 202 may comprise one or more edges 219 formed by the wires 212 of the frame 202. The frame 202 can comprise edges 219 extending variable and/or different lengths. In some examples, the frame 202 can comprise one or more anchors 221 configured to facilitate attachment to one or more delivery devices (e.g., catheters), native anatomy, and/or other components of the stent 200.

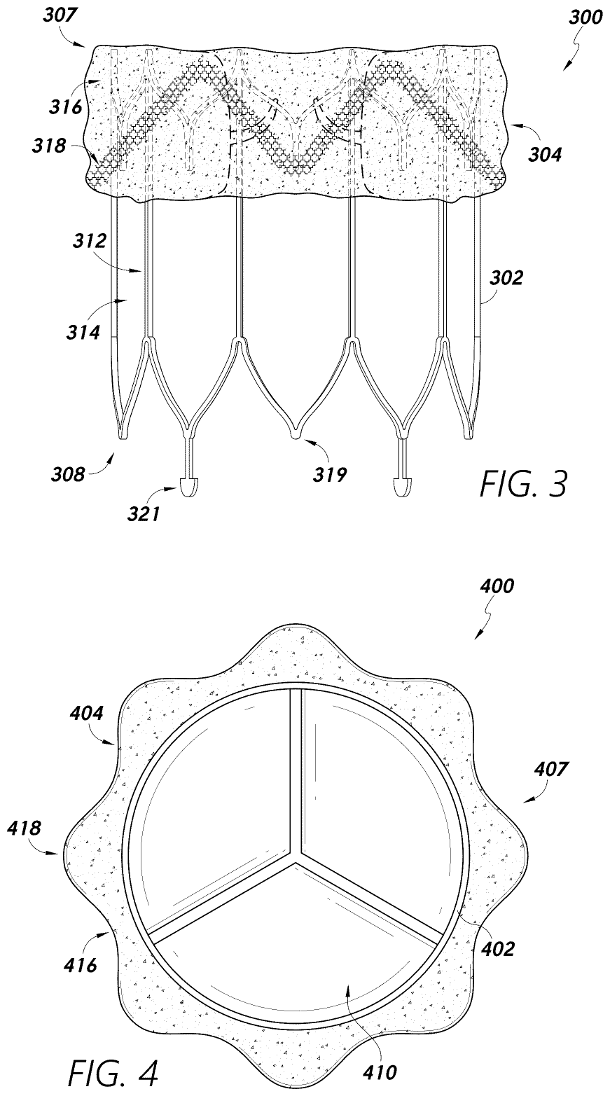

FIG. 3 provides a side view of an example stent 300 and/or docking device configured for docking one or more prosthetic valves and comprising a distal scaling element 304 at or near a distal end 307 of the stent 300 configured to increase surface friction at the distal end 307 of the stent in accordance with one or more examples. The stent 300 may further comprise a frame 302 having a generally cylindrical and/or tubular form. In some examples, the frame 302 may comprise a network of one or more wires 312 and/or cords forming one or more cells 314 between the wires 312.

The sealing element 304 may comprise a cloth and/or fabric having a variable thickness. For example, the sealing element 304 may comprise a first portion 316 having a relatively thin structure and/or may comprise a second portion 318 having a relatively thick structure. In some examples, the second portion 318 may extend from the first portion 316. The first portion 316 may comprise a different material than the second portion 318 and/or the second portion 318 may comprise a thicker and/or denser portion of a common material of the first portion 316.

The second portion 318 may comprise any suitable pattern and/or form. In the example shown in FIG. 3, the second portion 318 may comprise a zig-zag form and/or may have a repeating form across the circumference of the sealing element 304. The first portion 316 may extend across portions of the sealing element 304 between the second portion 318.

The second portion 318 and/or first portion 316 may comprise a cloth and/or fabric having a generally thick and/or thin structure and/or may increase a diameter of the frame 302 and/or may extend from the frame 302. The sealing element 304 may be configured to improve sealing of the stent 300 at the distal end 307. In some examples, the sealing element 304 may have a fibrous and/or fuzzy texture to increase friction between the stent 300 and a blood vessel and/or blood vessel junction (e.g., at or near the RA/IVC junction).

While the stent 300 is shown comprising a sealing element 304 only at the distal end 307 of the stent 300, the stent 300 may comprise multiple sealing elements 304 and/or the sealing element 304 may be configured to extend further along the frame 302. In some examples, the sealing element 304 may be disposed at an outer surface of the frame 302. The sealing element 304 may have a ring and/or generally circular shape and/or may be configured to extend along a full circumference of the frame 302.

The sealing element 304 may be configured to extend at least partially over a leading end of the frame 302 and/or to extend onto an inner surface of the frame 302. In some examples, the sealing element 304 may be configured to increase friction and/or grip with a prosthetic valve placed into an inner lumen of the frame 302. For example, the scaling element 304 may increase a width of the stent 300 and/or may be configured to extend from the inner surface of the frame 302 to provide a contact point for one or more prosthetic valves placed into the frame 302. The sealing element 304 may be configured to pinch the prosthetic valve to securely hold the prosthetic valve.

The stent 300 may be configured for delivery and/or placement at an SVC and/or IVC of a heart. For example, the stent 300 may be configured for placement at or near an in-flow junction of the SVC and/or IVC to the right atrium of the heart. The stent 300 may be configured for antegrade delivery (e.g., via the femoral vein) and/or retrograde delivery (e.g., via the subclavian and/or jugular vein).

The frame 302 may be at least partially composed of one or more shape memory alloys (e.g., Nitinol) and/or may be laser-cut into a desired form. In some examples, the frame 302 and/or sealing element 304 may have a generally flared, mushroom, and/or funnel shape and/or form at or near the distal end 307 of the stent 300. For example, the distal end 307 of the stent 300 may be flared and/or conical in shape to extend outward into the right atrium and/or to form a grip onto a rim of the right atrium. In some examples, the sealing element 304 may be configured to extend beyond the frame 302 and/or may form an extension of the frame 302 to extend further laterally and/or longitudinally relative to the frame 302. The frame 302 may further comprise a proximal end 308 having a straight or flared form.

The stent 300 may be configured to present minimal blockage of one or more hepatic veins and/or other branching blood vessels. For example, a heart may generally include one or more hepatic veins branching into the IVC at or near a junction between the IVC and the right atrium. In some examples, the sealing element 304 may not extend along a complete length of the frame 302. For example, the sealing element 304 may extend from the distal end 307 of the stent 300 to a midsection and/or midpoint of the stent 300. The stent 300 may be configured such that, when implanted at the junction between the IVC and the right atrium, the sealing element 304 may not extend over the one or more hepatic veins and/or branching blood vessels. In some examples, the frame 302 may at least partially extend over the hepatic veins and/or branching blood vessels and/or may allow blood flow from the branching blood vessels through cells 314 of the frame 302.

In some examples, the stent 300 may comprise one or more radiopaque markers configured to facilitate delivery and/or use of the stent 300. For example, one or more markers may be disposed along the sealing element 304 to assist with aligning and/or docking one or more prosthetic valves at the stent 300 and/or sealing element 304. In another example, the stent 300 may comprise one or more markers at the frame 302 and/or scaling element 304 at or near the distal end 307 to facilitate positioning of the stent 300 and/or distal end 307 at the atrium junction and/or other anatomy.

In some examples, the stent 300 may be configured to cause native anatomy to conform to a size and/or shape of the stent 300. For example, the frame 302, scaling element 304, and/or distal end 307 of the stent 300 may be configured to press outwardly against the native tissue to create a larger opening to support a prosthetic valve. In some examples, the stent 300 may comprise one or more bulbs configured to press outwardly against walls of a blood vessel and/or may be configured to correspondingly press walls of the blood vessel outwardly. The term “bulb” is used herein in accordance with its plain and ordinary meaning and may refer to any outward- and/or inward-extending portion of a frame 302. In some examples, the frame 302 may increase in diameter at the bulb and/or may decrease in diameter on either side of the bulb.

The frame 302 may be coupled to the sealing element 304 and/or may be configured to mate with the sealing element 304. In some examples, one or more sutures and/or other attachment features (e.g., clips, hooks, notches, stitches, adhesives, etc.) may be used to form an attachment between the frame 302 and the sealing element 304.

The stent 300 may comprise various components and/or features at the proximal end 308 of the frame 302. For example, the frame 302 may comprise one or more edges 319 formed by the wires 312 of the frame 302. The frame 302 can comprise edges 319 extending variable and/or different lengths. In some examples, the frame 302 can comprise one or more anchors 321 configured to facilitate attachment to one or more delivery devices (e.g., catheters), native anatomy, and/or other components of the stent 300.

FIG. 4 provides a top view of an example stent 400 and/or docking device configured for docking one or more prosthetic valves and comprising a distal scaling element 404 at or near a distal end of the stent 400 configured to increase surface friction at the distal end of the stent in accordance with one or more examples. The stent 400 may further comprise a frame 402 having a generally cylindrical and/or tubular form. In some examples, the frame 402 may comprise a network of one or more wires and/or cords forming one or more cells between the wires.

The sealing element 404 may comprise a cloth and/or fabric having a variable thickness. For example, the sealing element 404 may comprise a first portion 416 having a relatively thin structure and/or may comprise a second portion 418 having a relatively thick structure. In some examples, the second portion 418 may extend from the first portion 416. The first portion 416 may comprise a different material than the second portion 418 and/or the second portion 418 may comprise a thicker and/or denser portion of a common material of the first portion 416.

The second portion 418 may comprise any suitable pattern and/or form. In the example shown in FIG. 4, the second portion 418 may comprise one or more longitudinally extending portions and/or columns extending along a length of the frame 402. In some examples, areas of the second portion 418 may extend generally diagonally along a length of the frame 402. A thickness and/or width of the sealing element 404 may increase and/or decrease gradually between areas of the first portion 416 and areas of the second portion 318. The sealing element 404 may comprise a repeating pattern and/or areas of the first portion 416 may be disposed on either side by areas of the second portion 418.

The sealing element 404, first portion 416, and/or second portion 418 may comprise any suitable material(s), which can include tissues, polymers, polyethylene terephthalate (PET), polyester, cotton, and/or other suitable materials. The sealing element 404 may comprise an outer jacket enclosing and/or surround at least a portion (e.g., midsection and/or distal end 407) of the frame 402. The first portion 416 may extend across portions of the sealing element 404 between the second portion 418.

The second portion 418 and/or first portion 416 may comprise a cloth and/or fabric having a generally thick and/or thin structure and/or may increase a diameter of the frame 402 and/or may extend from the frame 402. The sealing element 404 may be configured to improve sealing of the stent 400 at the distal end. In some examples, the sealing element 404 may have a fibrous and/or fuzzy texture to increase friction between the stent 400 and a blood vessel and/or blood vessel junction (e.g., at or near the RA/IVC junction).

While the stent 400 is shown comprising a sealing element 404 only at the distal end 407 of the stent 400, the stent 400 may comprise multiple sealing elements 404 and/or the sealing element 404 may be configured to extend further along the frame 402. In some examples, the sealing element 404 may be disposed at an outer surface of the frame 402. The sealing element 404 may have a ring and/or generally circular shape and/or may be configured to extend along a full circumference of the frame 402.

The sealing element 404 may be configured to extend at least partially over a leading end of the frame 402 and/or to extend onto an inner surface of the frame 402. In some examples, the sealing element 404 may be configured to increase friction and/or grip with a prosthetic valve placed into an inner lumen 410 of the frame 402. For example, the sealing element 404 may increase a width of the stent 400 and/or may be configured to extend from the inner surface of the frame 402 to provide a contact point for one or more prosthetic valves placed into the frame 402. The sealing element 404 may be configured to pinch the prosthetic valve to securely hold the prosthetic valve.

The stent 400 may be configured for delivery and/or placement at an SVC and/or IVC of a heart. For example, the stent 400 may be configured for placement at or near an in-flow junction of the SVC and/or IVC to the right atrium of the heart. The stent 400 may be configured for antegrade delivery (e.g., via the femoral vein) and/or retrograde delivery (e.g., via the subclavian and/or jugular vein).

The frame 402 may be at least partially composed of one or more shape memory alloys (e.g., Nitinol) and/or may be laser-cut into a desired form. In some examples, the frame 402 and/or sealing element 404 may have a generally flared, mushroom, and/or funnel shape and/or form at or near the distal end 407 of the stent 400. For example, the distal end 407 of the stent 400 may be flared and/or conical in shape to extend outward into the right atrium and/or to form a grip onto a rim of the right atrium. In some examples, the sealing element 404 may be configured to extend beyond the frame 402 and/or may form an extension of the frame 402 to extend further laterally and/or longitudinally relative to the frame 402. The frame 402 may further comprise a proximal end having a straight or flared form.

The stent 400 may be configured to present minimal blockage of one or more hepatic veins and/or other branching blood vessels. For example, a heart may generally include one or more hepatic veins branching into the IVC at or near a junction between the IVC and the right atrium. In some examples, the sealing element 404 may not extend along a complete length of the frame 402. For example, the sealing element 404 may extend from the distal end 407 of the stent 400 to a midsection and/or midpoint of the stent 400. The stent 400 may be configured such that, when implanted at the junction between the IVC and the right atrium, the sealing element 404 may not extend over the one or more hepatic veins and/or branching blood vessels. In some examples, the frame 402 may at least partially extend over the hepatic veins and/or branching blood vessels and/or may allow blood flow from the branching blood vessels through cells of the frame 402.

In some examples, the stent 400 may comprise one or more radiopaque markers configured to facilitate delivery and/or use of the stent 400. For example, one or more markers may be disposed along the sealing element 404 to assist with aligning and/or docking one or more prosthetic valves at the stent 400 and/or sealing element 404. In another example, the stent 400 may comprise one or more markers at the frame 402 and/or scaling element 404 at or near the distal end 407 to facilitate positioning of the stent 400 and/or distal end 407 at the atrium junction and/or other anatomy.

In some examples, the stent 400 may be configured to cause native anatomy to conform to a size and/or shape of the stent 400. For example, the frame 402, sealing element 404, and/or distal end 407 of the stent 400 may be configured to press outwardly against the native tissue to create a larger opening to support a prosthetic valve. In some examples, the stent 400 may comprise one or more bulbs configured to press outwardly against walls of a blood vessel and/or may be configured to correspondingly press walls of the blood vessel outwardly. The term “bulb” is used herein in accordance with its plain and ordinary meaning and may refer to any outward- and/or inward-extending portion of a frame 402. In some examples, the frame 402 may increase in diameter at the bulb and/or may decrease in diameter on either side of the bulb.

The frame 402 may be coupled to the sealing element 404 and/or may be configured to mate with the sealing element 404. In some examples, one or more sutures and/or other attachment features (e.g., clips, hooks, notches, stitches, adhesives, etc.) may be used to form an attachment between the frame 402 and the sealing element 404.

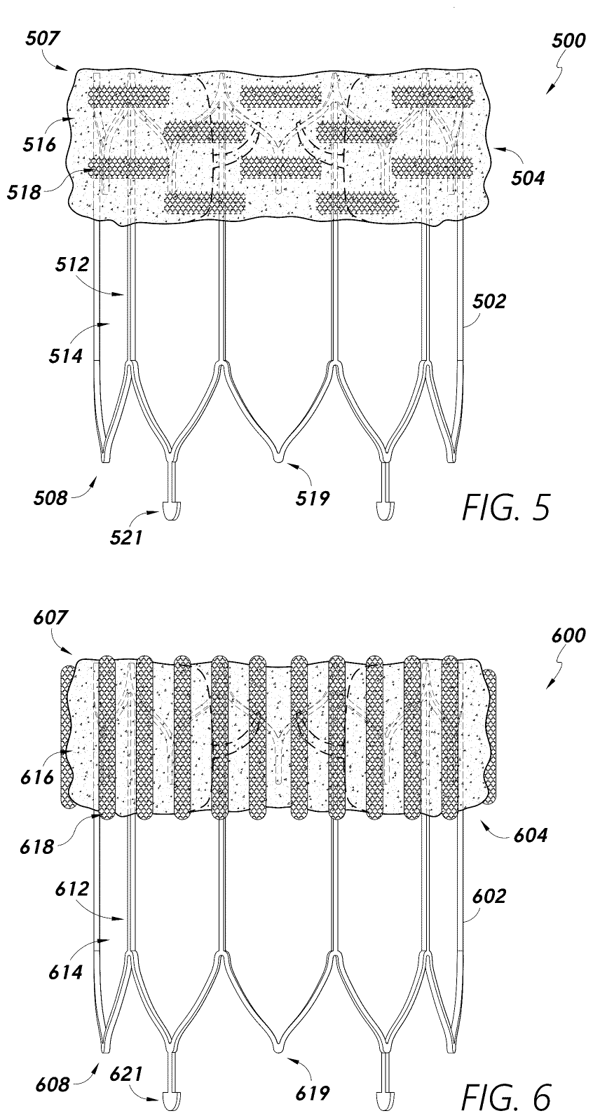

FIG. 5 provides a side view of an example stent 500 and/or docking device configured for docking one or more prosthetic valves and comprising a distal scaling element 504 at or near a distal end 507 of the stent 500 configured to increase surface friction at the distal end 507 of the stent in accordance with one or more examples. The stent 500 may further comprise a frame 502 having a generally cylindrical and/or tubular form. In some examples, the frame 502 may comprise a network of one or more wires 512 and/or cords forming one or more cells 514 between the wires 512.

The sealing element 504 may comprise a cloth and/or fabric having a variable thickness. For example, the sealing element 504 may comprise a first portion 516 having a relatively thin structure and/or may comprise one or more second portions 518 having a relatively thick structure. In some examples, the second portions 518 may extend from the first portion 516. The first portion 516 may comprise a different material than the second portion 518 and/or the second portions 518 may comprise a thicker and/or denser portion of a common material of the first portion 516.

The second portions 518 may be detached from each other and/or may be arranged along the first portion 516 in any suitable pattern and/or form. In the example shown in FIG. 5, the second portions 518 comprise rectangular and/or ovular shapes (e.g., bricks) disposed at various positions along the first portion 516, however the second portions 518 can have different shapes. The second portions 518 may be aligned in any suitable manner. In some examples, the second portions 518 may form multiple rows along a length of the frame 502 and/or first portion 516 and/or second portions 518 may be staggered row-to-row, similar to a brick formation. The first portion 516 may extend across portions of the sealing element 504 between the second portions 518.

The second portion 518 and/or first portion 516 may comprise a cloth and/or fabric having a generally thick and/or thin structure and/or may increase a diameter of the frame 502 and/or may extend from the frame 502. The sealing element 504 may be configured to improve sealing of the stent 500 at the distal end 507. In some examples, the sealing element 504 may have a fibrous and/or fuzzy texture to increase friction between the stent 500 and a blood vessel and/or blood vessel junction (e.g., at or near the RA/IVC junction).

While the stent 500 is shown comprising a sealing element 504 only at the distal end 507 of the stent 500, the stent 500 may comprise multiple sealing elements 504 and/or the sealing element 504 may be configured to extend further along the frame 502. In some examples, the sealing element 504 may be disposed at an outer surface of the frame 502. The sealing element 504 may have a ring and/or generally circular shape and/or may be configured to extend along a full circumference of the frame 502.

The sealing element 504 may be configured to extend at least partially over a leading end of the frame 502 and/or to extend onto an inner surface of the frame 502. In some examples, the sealing element 504 may be configured to increase friction and/or grip with a prosthetic valve placed into an inner lumen of the frame. For example, the sealing element 504 may increase a width of the stent 500 and/or may be configured to extend from the inner surface of the frame to provide a contact point for one or more prosthetic valves placed into the frame 502. The sealing element 504 may be configured to pinch the prosthetic valve to securely hold the prosthetic valve.

The stent 500 may be configured for delivery and/or placement at an SVC and/or IVC of a heart. For example, the stent 500 may be configured for placement at or near an in-flow junction of the SVC and/or IVC to the right atrium of the heart. The stent 500 may be configured for antegrade delivery (e.g., via the femoral vein) and/or retrograde delivery (e.g., via the subclavian and/or jugular vein).

The frame 502 may be at least partially composed of one or more shape memory alloys (e.g., Nitinol) and/or may be laser-cut into a desired form. In some examples, the frame 502 and/or sealing element 504 may have a generally flared, mushroom, and/or funnel shape and/or form at or near the distal end 507 of the stent 500. For example, the distal end 507 of the stent 500 may be flared and/or conical in shape to extend outward into the right atrium and/or to form a grip onto a rim of the right atrium. In some examples, the sealing element 504 may be configured to extend beyond the frame 502 and/or may form an extension of the frame 502 to extend further laterally and/or longitudinally relative to the frame 502. The frame 502 may further comprise a proximal end 508 having a straight or flared form.

The stent 500 may be configured to present minimal blockage of one or more hepatic veins and/or other branching blood vessels. For example, a heart may generally include one or more hepatic veins branching into the IVC at or near a junction between the IVC and the right atrium. In some examples, the sealing element 504 may not extend along a complete length of the frame 502. For example, the sealing element 504 may extend from the distal end 507 of the stent 500 to a midsection and/or midpoint of the stent 500. The stent 500 may be configured such that, when implanted at the junction between the IVC and the right atrium, the sealing element 504 may not extend over the one or more hepatic veins and/or branching blood vessels. In some examples, the frame 502 may at least partially extend over the hepatic veins and/or branching blood vessels and/or may allow blood flow from the branching blood vessels through cells 514 of the frame 502.

In some examples, the stent 500 may comprise one or more radiopaque markers configured to facilitate delivery and/or use of the stent 500. For example, one or more markers may be disposed along the sealing element 504 to assist with aligning and/or docking one or more prosthetic valves at the stent 500 and/or sealing element 504. In another example, the stent 500 may comprise one or more markers at the frame 502 and/or sealing element 504 at or near the distal end 507 to facilitate positioning of the stent 500 and/or distal end 507 at the atrium junction and/or other anatomy.

In some examples, the stent 500 may be configured to cause native anatomy to conform to a size and/or shape of the stent 500. For example, the frame 502, sealing element 504, and/or distal end 507 of the stent 500 may be configured to press outwardly against the native tissue to create a larger opening to support a prosthetic valve. In some examples, the stent 500 may comprise one or more bulbs configured to press outwardly against walls of a blood vessel and/or may be configured to correspondingly press walls of the blood vessel outwardly. The term “bulb” is used herein in accordance with its plain and ordinary meaning and may refer to any outward- and/or inward-extending portion of a frame 502. In some examples, the frame 502 may increase in diameter at the bulb and/or may decrease in diameter on either side of the bulb.

The frame 502 may be coupled to the sealing element 504 and/or may be configured to mate with the sealing element 504. In some examples, one or more sutures and/or other attachment features (e.g., clips, hooks, notches, stitches, adhesives, etc.) may be used to form an attachment between the frame 502 and the scaling element 504.

The stent 500 may comprise various components and/or features at the proximal end 508 of the frame 502. For example, the frame 502 may comprise one or more edges 519 formed by the wires 512 of the frame 502. The frame 502 can comprise edges 519 extending variable and/or different lengths. In some examples, the frame 502 can comprise one or more anchors 521 configured to facilitate attachment to one or more delivery devices (e.g., catheters), native anatomy, and/or other components of the stent 500.

FIG. 6 provides a side view of an example stent 600 and/or docking device configured for docking one or more prosthetic valves and comprising a distal scaling element 604 at or near a distal end 607 of the stent 600 configured to increase surface friction at the distal end 607 of the stent in accordance with one or more examples. The stent 600 may further comprise a frame 602 having a generally cylindrical and/or tubular form. In some examples, the frame 602 may comprise a network of one or more wires 612 and/or cords forming one or more cells 614 between the wires 612.

The sealing element 604 may comprise a cloth and/or fabric having a variable thickness. For example, the sealing element 604 may comprise a first portion 616 having a relatively thin structure and/or may comprise one or more second portions 618 having a relatively thick structure. In some examples, the second portions 618 may extend from the first portion 616. The first portion 616 may comprise a different material than the second portion 618 and/or the second portions 618 may comprise a thicker and/or denser portion of a common material of the first portion 616.

The second portions 618 may comprise any suitable pattern and/or form. In the example shown in FIG. 6, the second portions 618 may comprise one or more longitudinally extending portions and/or columns extending along a length of the frame 602. In some examples, second portions 618 may extend generally diagonally along a length of the frame 602. A thickness and/or width of the sealing element 604 may increase and/or decrease gradually between areas of the first portion 616 and areas of the second portion 318. The sealing element 604 may comprise a repeating pattern and/or areas of the first portion 616 may be disposed on either side by areas of the second portion 618.

The sealing element 604, first portion 616, and/or second portion 618 may comprise any suitable material(s), which can include tissues, polymers, PET, polyester, cotton, and/or other suitable materials. The sealing element 604 may comprise an outer jacket enclosing and/or surround at least a portion (e.g., midsection and/or distal end 607) of the frame 602. The first portion 616 may extend across portions of the sealing element 604 between the second portion 618.

The second portion 618 and/or first portion 616 may comprise a cloth and/or fabric having a generally thick and/or thin structure and/or may increase a diameter of the frame 602 and/or may extend from the frame 602. The sealing element 604 may be configured to improve sealing of the stent 600 at the distal end 607. In some examples, the sealing element 604 may have a fibrous and/or fuzzy texture to increase friction between the stent 600 and a blood vessel and/or blood vessel junction (e.g., at or near the RA/IVC junction).

While the stent 600 is shown comprising a sealing element 604 only at the distal end 607 of the stent 600, the stent 600 may comprise multiple sealing elements 604 and/or the sealing element 604 may be configured to extend further along the frame 602. In some examples, the sealing element 604 may be disposed at an outer surface of the frame 602. The sealing element 604 may have a ring and/or generally circular shape and/or may be configured to extend along a full circumference of the frame 602.

The sealing element 604 may be configured to extend at least partially over a leading end of the frame 602 and/or to extend onto an inner surface of the frame 602. In some examples, the sealing element 604 may be configured to increase friction and/or grip with a prosthetic valve placed into an inner lumen of the frame. For example, the sealing element 604 may increase a width of the stent 600 and/or may be configured to extend from the inner surface of the frame to provide a contact point for one or more prosthetic valves placed into the frame 602. The sealing element 604 may be configured to pinch the prosthetic valve to securely hold the prosthetic valve.

The stent 600 may be configured for delivery and/or placement at an SVC and/or IVC of a heart. For example, the stent 600 may be configured for placement at or near an in-flow junction of the SVC and/or IVC to the right atrium of the heart. The stent 600 may be configured for antegrade delivery (e.g., via the femoral vein) and/or retrograde delivery (e.g., via the subclavian and/or jugular vein).

The frame 602 may be at least partially composed of one or more shape memory alloys (e.g., Nitinol) and/or may be laser-cut into a desired form. In some examples, the frame 602 and/or sealing element 604 may have a generally flared, mushroom, and/or funnel shape and/or form at or near the distal end 607 of the stent 600. For example, the distal end 607 of the stent 600 may be flared and/or conical in shape to extend outward into the right atrium and/or to form a grip onto a rim of the right atrium. In some examples, the scaling element 604 may be configured to extend beyond the frame 602 and/or may form an extension of the frame 602 to extend further laterally and/or longitudinally relative to the frame 602. The frame 602 may further comprise a proximal end 608 having a straight or flared form.

The stent 600 may be configured to present minimal blockage of one or more hepatic veins and/or other branching blood vessels. For example, a heart may generally include one or more hepatic veins branching into the IVC at or near a junction between the IVC and the right atrium. In some examples, the sealing element 604 may not extend along a complete length of the frame 602. For example, the sealing element 604 may extend from the distal end 607 of the stent 600 to a midsection and/or midpoint of the stent 600. The stent 600 may be configured such that, when implanted at the junction between the IVC and the right atrium, the sealing element 604 may not extend over the one or more hepatic veins and/or branching blood vessels. In some examples, the frame 602 may at least partially extend over the hepatic veins and/or branching blood vessels and/or may allow blood flow from the branching blood vessels through cells 614 of the frame 602.

In some examples, the stent 600 may comprise one or more radiopaque markers configured to facilitate delivery and/or use of the stent 600. For example, one or more markers may be disposed along the sealing element 604 to assist with aligning and/or docking one or more prosthetic valves at the stent 600 and/or sealing element 604. In another example, the stent 600 may comprise one or more markers at the frame 602 and/or scaling element 604 at or near the distal end 607 to facilitate positioning of the stent 600 and/or distal end 607 at the atrium junction and/or other anatomy.

In some examples, the stent 600 may be configured to cause native anatomy to conform to a size and/or shape of the stent 600. For example, the frame 602, sealing element 604, and/or distal end 607 of the stent 600 may be configured to press outwardly against the native tissue to create a larger opening to support a prosthetic valve. In some examples, the stent 600 may comprise one or more bulbs configured to press outwardly against walls of a blood vessel and/or may be configured to correspondingly press walls of the blood vessel outwardly. The term “bulb” is used herein in accordance with its plain and ordinary meaning and may refer to any outward- and/or inward-extending portion of a frame 602. In some examples, the frame 602 may increase in diameter at the bulb and/or may decrease in diameter on either side of the bulb.

The frame 602 may be coupled to the sealing element 604 and/or may be configured to mate with the sealing element 604. In some examples, one or more sutures and/or other attachment features (e.g., clips, hooks, notches, stitches, adhesives, etc.) may be used to form an attachment between the frame 602 and the sealing element 604.

The stent 600 may comprise various components and/or features at the proximal end 608 of the frame 602. For example, the frame 602 may comprise one or more edges 619 formed by the wires 612 of the frame 602. The frame 602 can comprise edges 619 extending variable and/or different lengths. In some examples, the frame 602 can comprise one or more anchors 621 configured to facilitate attachment to one or more delivery devices (e.g., catheters), native anatomy, and/or other components of the stent 600.

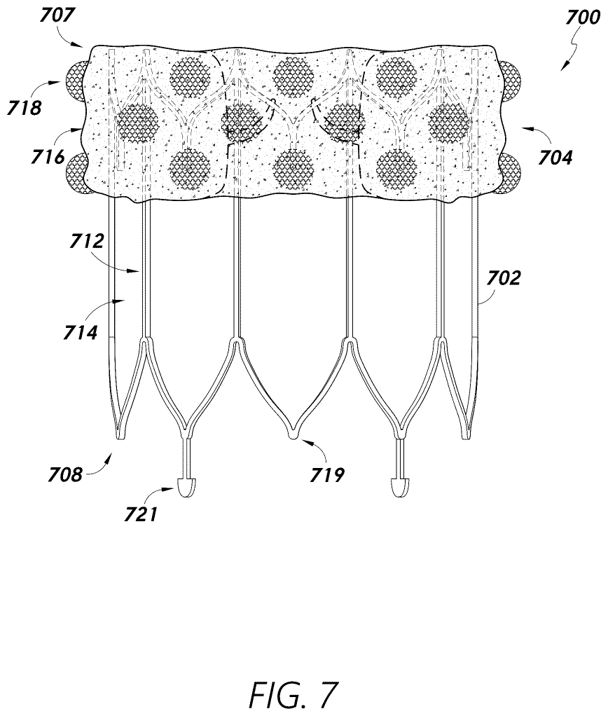

FIG. 7 provides a side view of an example stent 700 and/or docking device configured for docking one or more prosthetic valves and comprising a distal sealing element 704 at or near a distal end 707 of the stent 700 configured to increase surface friction at the distal end 707 of the stent in accordance with one or more examples. The stent 700 may further comprise a frame 702 having a generally cylindrical and/or tubular form. In some examples, the frame 702 may comprise a network of one or more wires 712 and/or cords forming one or more cells 714 between the wires 712.

The sealing element 704 may comprise a cloth and/or fabric having a variable thickness. For example, the sealing element 704 may comprise a first portion 716 having a relatively thin structure and/or may comprise one or more second portions 718 having a relatively thick structure. In some examples, the second portions 718 may extend from the first portion 716. The first portion 716 may comprise a different material than the second portion 718 and/or the second portions 718 may comprise a thicker and/or denser portion of a common material of the first portion 716.

The second portions 718 may be detached from each other and/or may be arranged along the first portion 716 in any suitable pattern and/or form. In the example shown in FIG. 7, the second portions 718 comprise half dome and/or semi-spherical shape disposed at various positions along the first portion 716, however the second portions 718 can have different shapes. The second portions 718 may be aligned in any suitable manner. In some examples, the second portions 718 may form multiple rows along a length of the frame 702 and/or first portion 716 and/or second portions 718 may be staggered row-to-row, similar to a brick formation. The first portion 716 may extend across portions of the sealing element 704 between the second portions 718.

The second portion 718 and/or first portion 716 may comprise a cloth and/or fabric having a generally thick and/or thin structure and/or may increase a diameter of the frame 702 and/or may extend from the frame 702. The sealing element 704 may be configured to improve sealing of the stent 700 at the distal end 707. In some examples, the scaling element 704 may have a fibrous and/or fuzzy texture to increase friction between the stent 700 and a blood vessel and/or blood vessel junction (e.g., at or near the RA/IVC junction).

While the stent 700 is shown comprising a sealing element 704 only at the distal end 707 of the stent 700, the stent 700 may comprise multiple scaling elements 704 and/or the sealing element 704 may be configured to extend further along the frame 702. In some examples, the scaling element 704 may be disposed at an outer surface of the frame 702. The sealing element 704 may have a ring and/or generally circular shape and/or may be configured to extend along a full circumference of the frame 702.

The sealing element 704 may be configured to extend at least partially over a leading end of the frame 702 and/or to extend onto an inner surface of the frame 702. In some examples, the sealing element 704 may be configured to increase friction and/or grip with a prosthetic valve placed into an inner lumen of the frame 702. For example, the sealing element 704 may increase a width of the stent 700 and/or may be configured to extend from the inner surface of the frame 702 to provide a contact point for one or more prosthetic valves placed into the frame 702. The sealing element 704 may be configured to pinch the prosthetic valve to securely hold the prosthetic valve.

The stent 700 may be configured for delivery and/or placement at an SVC and/or IVC of a heart. For example, the stent 700 may be configured for placement at or near an in-flow junction of the SVC and/or IVC to the right atrium of the heart. The stent 700 may be configured for antegrade delivery (e.g., via the femoral vein) and/or retrograde delivery (e.g., via the subclavian and/or jugular vein).

The frame 702 may be at least partially composed of one or more shape memory alloys (e.g., Nitinol) and/or may be laser-cut into a desired form. In some examples, the frame 702 and/or sealing element 704 may have a generally flared, mushroom, and/or funnel shape and/or form at or near the distal end 707 of the stent 700. For example, the distal end 707 of the stent 700 may be flared and/or conical in shape to extend outward into the right atrium and/or to form a grip onto a rim of the right atrium. In some examples, the sealing element 704 may be configured to extend beyond the frame 702 and/or may form an extension of the frame 702 to extend further laterally and/or longitudinally relative to the frame 702. The frame 702 may further comprise a proximal end 708 having a straight or flared form.

The stent 700 may be configured to present minimal blockage of one or more hepatic veins and/or other branching blood vessels. For example, a heart may generally include one or more hepatic veins branching into the IVC at or near a junction between the IVC and the right atrium. In some examples, the sealing element 704 may not extend along a complete length of the frame 702. For example, the sealing element 704 may extend from the distal end 707 of the stent 700 to a midsection and/or midpoint of the stent 700. The stent 700 may be configured such that, when implanted at the junction between the IVC and the right atrium, the sealing element 704 may not extend over the one or more hepatic veins and/or branching blood vessels. In some examples, the frame 702 may at least partially extend over the hepatic veins and/or branching blood vessels and/or may allow blood flow from the branching blood vessels through cells 714 of the frame 702.

In some examples, the stent 700 may comprise one or more radiopaque markers configured to facilitate delivery and/or use of the stent 700. For example, one or more markers may be disposed along the sealing element 704 to assist with aligning and/or docking one or more prosthetic valves at the stent 700 and/or sealing element 704. In another example, the stent 700 may comprise one or more markers at the frame 702 and/or scaling element 704 at or near the distal end 707 to facilitate positioning of the stent 700 and/or distal end 707 at the atrium junction and/or other anatomy.

In some examples, the stent 700 may be configured to cause native anatomy to conform to a size and/or shape of the stent 700. For example, the frame 702, sealing element 704, and/or distal end 707 of the stent 700 may be configured to press outwardly against the native tissue to create a larger opening to support a prosthetic valve. In some examples, the stent 700 may comprise one or more bulbs configured to press outwardly against walls of a blood vessel and/or may be configured to correspondingly press walls of the blood vessel outwardly. The term “bulb” is used herein in accordance with its plain and ordinary meaning and may refer to any outward- and/or inward-extending portion of a frame 702. In some examples, the frame 702 may increase in diameter at the bulb and/or may decrease in diameter on either side of the bulb.

The frame 702 may be coupled to the sealing element 704 and/or may be configured to mate with the sealing element 704. In some examples, one or more sutures and/or other attachment features (e.g., clips, hooks, notches, stitches, adhesives, etc.) may be used to form an attachment between the frame 702 and the sealing element 704.

The stent 700 may comprise various components and/or features at the proximal end 708 of the frame 702. For example, the frame 702 may comprise one or more edges 719 formed by the wires 712 of the frame 702. The frame 702 can comprise edges 719 extending variable and/or different lengths. In some examples, the frame 702 can comprise one or more anchors 721 configured to facilitate attachment to one or more delivery devices (e.g., catheters), native anatomy, and/or other components of the stent 700.

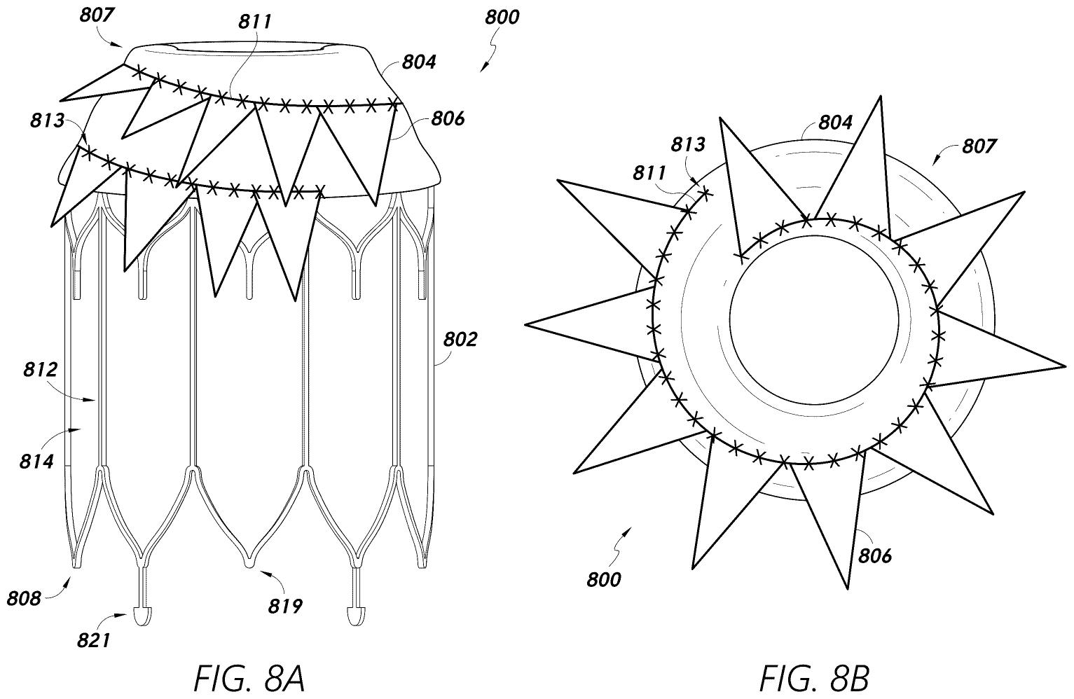

FIGS. 8A and 8B illustrate an example stent 800 and/or docking devices (e.g., docking stents and/or docking stations) configured for docking one or more prosthetic valves in accordance with one or more examples. FIG. 8A provides a side view of the stent 800 and FIG. 8B provides a top view of the stent 800. The stent 800 may comprise one or more sealing elements, which can include one or more flaps 806 (e.g., flags). The one or more flaps 806 may be configured to attach to the stent 800. For example, the one or more flaps 806 may be stitched, sewn, and/or otherwise coupled to a sealing portion 804 and/or frame 802 of the stent 800.

The one or more flaps 806 can be at least partially composed of fabric (e.g., PET, etc.) and/or any suitable materials. A flap 806 can have any suitable shape. In the example shown in FIGS. 8A and 8B, the one or more flaps 806 have a triangular shape, however other shapes may be suitable. In some examples, the one or more flaps 806 can have a variable width and/or can decrease in width from a fixed end (e.g., sutured end) of a flap 806 to a free end of the flap 806. The one or more flaps 806 may have a generally soft and/or flexible form and/or may be configured to be moved by blood flow around the stent 800. For example, blood flow along the one or more flaps 806 may cause the free ends of the one or more flaps 806 to extend away from the frame 802 and/or sealing portion 804. In some examples, the one or more flaps 806 may be configured to wrinkle and/or bunch in response to friction with anatomy around the stent 800 and/or may be configured to facilitate anchoring of the stent 800. The one or more flaps 806 may be coupled to an outer surface of the frame 802 and/or sealing portion 804 to allow the flaps 806 to interact with native anatomy around the stent 800.

In some examples, the one or more flaps 806 may be coupled in series around a circumference of the stent 800. The one or more flaps 806 may form a helical pattern along at least a portion of the stent 800. In some examples, the stent 800 may comprise a distal end 807 and/or distal portion having a generally tapered form. The one or more flaps 806 may be configured to extend at least partially along the distal end 807 and/or other portions of the stent 800. The stent 800 may have a generally cylindrical and/or tubular stent body.

The stent 800 may be configured for delivery and/or placement at an SVC and/or IVC of a heart. For example, the stent 800 may be configured for placement at or near an in-flow junction of the SVC and/or IVC to the right atrium of the heart. The stent 800 may be configured for antegrade delivery (e.g., via the femoral vein) and/or retrograde delivery (e.g., via the subclavian and/or jugular vein).

The frame 802 may be at least partially composed of one or more shape memory alloys (e.g., Nitinol) and/or may be laser-cut into a desired form. In some examples, the frame 802 and/or sealing element 804 may have a generally flared, mushroom, and/or funnel shape and/or form at or near the distal end 807 of the stent 800. For example, the distal end 807 of the stent 800 may be flared and/or conical in shape to extend outward into the right atrium and/or to form a grip onto a rim of the right atrium. In some examples, the scaling element 804 may be configured to extend beyond the frame 802 and/or may form an extension of the frame 802 to extend further laterally and/or longitudinally relative to the frame 802. The frame 802 may further comprise a proximal end 808 having a straight or flared form.

The stent 800 may be configured to present minimal blockage of one or more hepatic veins and/or other branching blood vessels. For example, a heart may generally include one or more hepatic veins branching into the IVC at or near a junction between the IVC and the right atrium. In some examples, the sealing element 804 may not extend along a complete length of the frame 802. For example, the sealing element 804 may extend from the distal end 807 of the stent 800 to a midsection and/or midpoint of the stent 800. The stent 800 may be configured such that, when implanted at the junction between the IVC and the right atrium, the sealing element 804 may not extend over the one or more hepatic veins and/or branching blood vessels. In some examples, the frame 802 may at least partially extend over the hepatic veins and/or branching blood vessels and/or may allow blood flow from the branching blood vessels through cells 814 of the frame 802.

In some examples, the stent 800 may comprise one or more radiopaque markers configured to facilitate delivery and/or use of the stent 800. For example, one or more markers may be disposed along the sealing element 804 to assist with aligning and/or docking one or more prosthetic valves at the stent 800 and/or sealing element 804. In another example, the stent 800 may comprise one or more markers at the frame 802 and/or sealing element 804 at or near the distal end 807 to facilitate positioning of the stent 800 and/or distal end 807 at the atrium junction and/or other anatomy.

In some examples, the stent 800 may be configured to cause native anatomy to conform to a size and/or shape of the stent 800. For example, the frame 802, sealing element 804, and/or distal end 807 of the stent 800 may be configured to press outwardly against the native tissue to create a larger opening to support a prosthetic valve. In some examples, the stent 800 may comprise one or more bulbs configured to press outwardly against walls of a blood vessel and/or may be configured to correspondingly press walls of the blood vessel outwardly. The term “bulb” is used herein in accordance with its plain and ordinary meaning and may refer to any outward- and/or inward-extending portion of a frame 802. In some examples, the frame 802 may increase in diameter at the bulb and/or may decrease in diameter on either side of the bulb.

The frame 802 may be coupled to the sealing element 804 and/or may be configured to mate with the sealing element 804. In some examples, one or more sutures 811 and/or other attachment features (e.g., clips, hooks, notches, stitches, adhesives, etc.) may be used to form an attachment between the frame 802 and the sealing element 804. For example a suture 811 may extend in a helical pattern around the sealing element 804 and/or stent 800 and/or may be stitched to the sealing element 804, stent 800, and/or one or more flaps 806 via one or more stiches 813.

The stent 800 may comprise various components and/or features at the proximal end 808 of the frame 802. For example, the frame 802 may comprise one or more edges 819 formed by the wires 812 of the frame 802. The frame 802 can comprise edges 819 extending variable and/or different lengths. In some examples, the frame 802 can comprise one or more anchors 821 configured to facilitate attachment to one or more delivery devices (e.g., catheters), native anatomy, and/or other components of the stent 800.

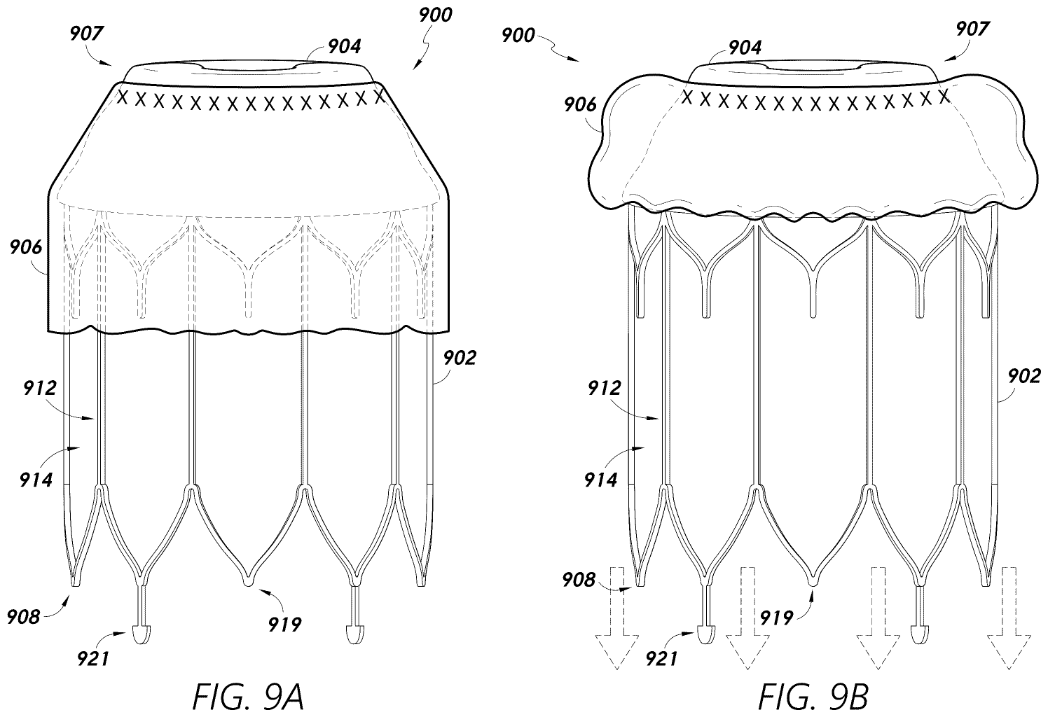

FIGS. 9A and 9B illustrate an example stent 900 and/or docking devices (e.g., docking stents and/or docking stations) configured for docking one or more prosthetic valves in accordance with one or more examples. FIG. 9A provides a side view of the stent 900 in a relaxed form and FIG. 9B provides a side view of the stent 900 in an expanded form. The stent 900 may comprise one or more sealing elements, which can include a skirt 906. The skirt 906 may be configured to attach to the stent 900. For example, the skirt 906 may be stitched, sewn, and/or otherwise coupled to a sealing portion 904 and/or frame 902 of the stent 900.

The skirt 906 can be at least partially composed of fabric (e.g., PET, etc.) and/or any suitable materials. In the example shown in FIGS. 9A and 9B, the skirt 906 may comprise a sheet of material with an opening in the center configured to align with a similar opening through the frame 902 and/or sealing portion 904. The skirt 906 may have a generally soft and/or flexible form and/or may be configured to be moved by blood flow around the stent 900. For example, blood flow along the skirt 906 and/or movement of the stent 900 may cause the free ends of the skirt 906 to extend towards the coupled end of the skirt 906. As a result, the skirt 906 may be configured to balloon, parachute, and/or expand outwardly and/or trap blood flow inside the skirt 906, as shown in FIG. 9B. Expansion of the skirt 906 may be configured to increase anchoring and/or friction between the skirt 906 and native anatomy around the stent 900. In some examples, the skirt 906 may be configured to wrinkle and/or bunch in response to friction with anatomy around the stent 900 and/or may be configured to facilitate anchoring of the stent 900. The skirt 906 may be coupled to an outer surface of the frame 902 and/or sealing portion 904 to allow the skirt 906 to interact with native anatomy around the stent 900.

The stent 900 may be configured for delivery and/or placement at an SVC and/or IVC of a heart. For example, the stent 900 may be configured for placement at or near an in-flow junction of the SVC and/or IVC to the right atrium of the heart. The stent 900 may be configured for antegrade delivery (e.g., via the femoral vein) and/or retrograde delivery (e.g., via the subclavian and/or jugular vein).

The frame 902 may be at least partially composed of one or more shape memory alloys (e.g., Nitinol) and/or may be laser-cut into a desired form. In some examples, the frame 902 and/or sealing element 904 may have a generally flared, mushroom, and/or funnel shape and/or form at or near the distal end 907 of the stent 900. For example, the distal end 907 of the stent 900 may be flared and/or conical in shape to extend outward into the right atrium and/or to form a grip onto a rim of the right atrium. In some examples, the sealing element 904 may be configured to extend beyond the frame 902 and/or may form an extension of the frame 902 to extend further laterally and/or longitudinally relative to the frame 902. The frame 902 may further comprise a proximal end 908 having a straight or flared form.

The stent 900 may be configured to present minimal blockage of one or more hepatic veins and/or other branching blood vessels. For example, a heart may generally include one or more hepatic veins branching into the IVC at or near a junction between the IVC and the right atrium. In some examples, the sealing element 904 may not extend along a complete length of the frame 902. For example, the sealing element 904 may extend from the distal end 907 of the stent 900 to a midsection and/or midpoint of the stent 900. The stent 900 may be configured such that, when implanted at the junction between the IVC and the right atrium, the sealing element 904 may not extend over the one or more hepatic veins and/or branching blood vessels. In some examples, the frame 902 may at least partially extend over the hepatic veins and/or branching blood vessels and/or may allow blood flow from the branching blood vessels through cells 914 of the frame 902.

In some examples, the stent 900 may comprise one or more radiopaque markers configured to facilitate delivery and/or use of the stent 900. For example, one or more markers may be disposed along the sealing element 904 to assist with aligning and/or docking one or more prosthetic valves at the stent 900 and/or sealing element 904. In another example, the stent 900 may comprise one or more markers at the frame 902 and/or sealing element 904 at or near the distal end 907 to facilitate positioning of the stent 900 and/or distal end 907 at the atrium junction and/or other anatomy.

In some examples, the stent 900 may be configured to cause native anatomy to conform to a size and/or shape of the stent 900. For example, the frame 902, sealing element 904, and/or distal end 907 of the stent 900 may be configured to press outwardly against the native tissue to create a larger opening to support a prosthetic valve. In some examples, the stent 900 may comprise one or more bulbs configured to press outwardly against walls of a blood vessel and/or may be configured to correspondingly press walls of the blood vessel outwardly. The term “bulb” is used herein in accordance with its plain and ordinary meaning and may refer to any outward- and/or inward-extending portion of a frame 902. In some examples, the frame 902 may increase in diameter at the bulb and/or may decrease in diameter on either side of the bulb.

The frame 902 may be coupled to the sealing element 904 and/or may be configured to mate with the sealing element 904. In some examples, one or more sutures and/or other attachment features (e.g., clips, hooks, notches, stitches, adhesives, etc.) may be used to form an attachment between the frame 902 and the sealing element 904.

The stent 900 may comprise various components and/or features at the proximal end 908 of the frame 902. For example, the frame 902 may comprise one or more edges 919 formed by the wires 912 of the frame 902. The frame 902 can comprise edges 919 extending variable and/or different lengths. In some examples, the frame 902 can comprise one or more anchors 921 configured to facilitate attachment to one or more delivery devices (e.g., catheters), native anatomy, and/or other components of the stent 900.

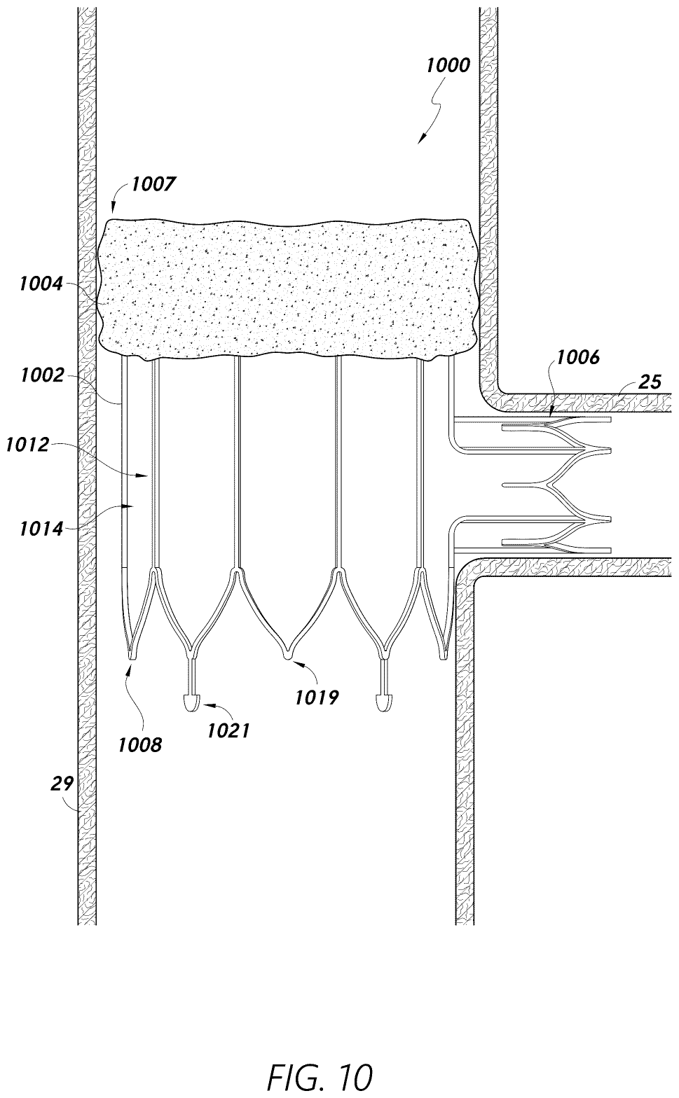

FIG. 10 illustrates an example stent 1000 and/or docking device (e.g., docking stent and/or docking station) configured for docking one or more prosthetic valves in accordance with one or more examples. The stent 1000 may comprise a frame 1002 and/or one or more sealing elements 1004 disposed at distal end 1007 of the stent 1000. The stent 1000 may further comprise one or more laterally extending protrusions 1006 and/or tubes configured to extend laterally from the frame 1002 and/or to anchor into one or more hepatic veins 25.

The stent 1000 may be configured for delivery and/or placement at an SVC and/or IVC of a heart. For example, the stent 1000 may be configured for placement at or near an in-flow junction of the SVC and/or IVC to the right atrium of the heart. The stent 1000 may be configured for antegrade delivery (e.g., via the femoral vein) and/or retrograde delivery (e.g., via the subclavian and/or jugular vein).

The frame 1002 may be at least partially composed of one or more shape memory alloys (e.g., Nitinol) and/or may be laser-cut into a desired form. In some examples, the frame 1002 and/or sealing element 1004 may have a generally flared, mushroom, and/or funnel shape and/or form at or near the distal end 1007 of the stent 1000. For example, the distal end 1007 of the stent 1000 may be flared and/or conical in shape to extend outward into the right atrium and/or to form a grip onto a rim of the right atrium. In some examples, the sealing element 1004 may be configured to extend beyond the frame 1002 and/or may form an extension of the frame 1002 to extend further laterally and/or longitudinally relative to the frame 1002. The frame 1002 may further comprise a proximal end 1008 having a straight or flared form.

The stent 1000 may be configured to present minimal blockage of one or more hepatic veins 25 and/or other branching blood vessels. For example, a heart may generally include one or more hepatic veins 25 branching into the IVC 10 at or near a junction between the IVC and the right atrium. In some examples, the sealing element 1004 may not extend along a complete length of the frame 1002. For example, the sealing element 1004 may extend from the distal end 1007 of the stent 1000 to a midsection and/or midpoint of the stent 1000. The stent 1000 may be configured such that, when implanted at the junction between the IVC and the right atrium, the sealing element 1004 may not extend over the one or more hepatic veins 25 and/or branching blood vessels. In some examples, the frame 1002 may at least partially extend over the hepatic veins 25 and/or branching blood vessels and/or may allow blood flow from the branching blood vessels through cells 1014 of the frame 1002.

In some examples, the stent 1000 may comprise one or more radiopaque markers configured to facilitate delivery and/or use of the stent 1000. For example, one or more markers may be disposed along the sealing element 1004 to assist with aligning and/or docking one or more prosthetic valves at the stent 1000 and/or sealing element 1004. In another example, the stent 1000 may comprise one or more markers at the frame 1002 and/or sealing element 1004 at or near the distal end 1007 to facilitate positioning of the stent 1000 and/or distal end 1007 at the atrium junction and/or other anatomy.

In some examples, the stent 1000 may be configured to cause native anatomy to conform to a size and/or shape of the stent 1000. For example, the frame 1002, scaling element 1004, and/or distal end 1007 of the stent 1000 may be configured to press outwardly against the native tissue to create a larger opening to support a prosthetic valve. In some examples, the stent 1000 may comprise one or more bulbs configured to press outwardly against walls of a blood vessel and/or may be configured to correspondingly press walls of the blood vessel outwardly. The term “bulb” is used herein in accordance with its plain and ordinary meaning and may refer to any outward- and/or inward-extending portion of a frame 1002. In some examples, the frame 1002 may increase in diameter at the bulb and/or may decrease in diameter on either side of the bulb.

The frame 1002 may be coupled to the sealing element 1004 and/or may be configured to mate with the sealing element 1004. In some examples, one or more sutures and/or other attachment features (e.g., clips, hooks, notches, stitches, adhesives, etc.) may be used to form an attachment between the frame 1002 and the sealing element 1004.

The stent 1000 may comprise various components and/or features at the proximal end 1008 of the frame 1002. For example, the frame 1002 may comprise one or more edges 1019 formed by the wires 1012 of the frame 1002. The frame 1002 can comprise edges 1019 extending variable and/or different lengths. In some examples, the frame 1002 can comprise one or more anchors 1021 configured to facilitate attachment to one or more delivery devices (e.g., catheters), native anatomy, and/or other components of the stent 1000.

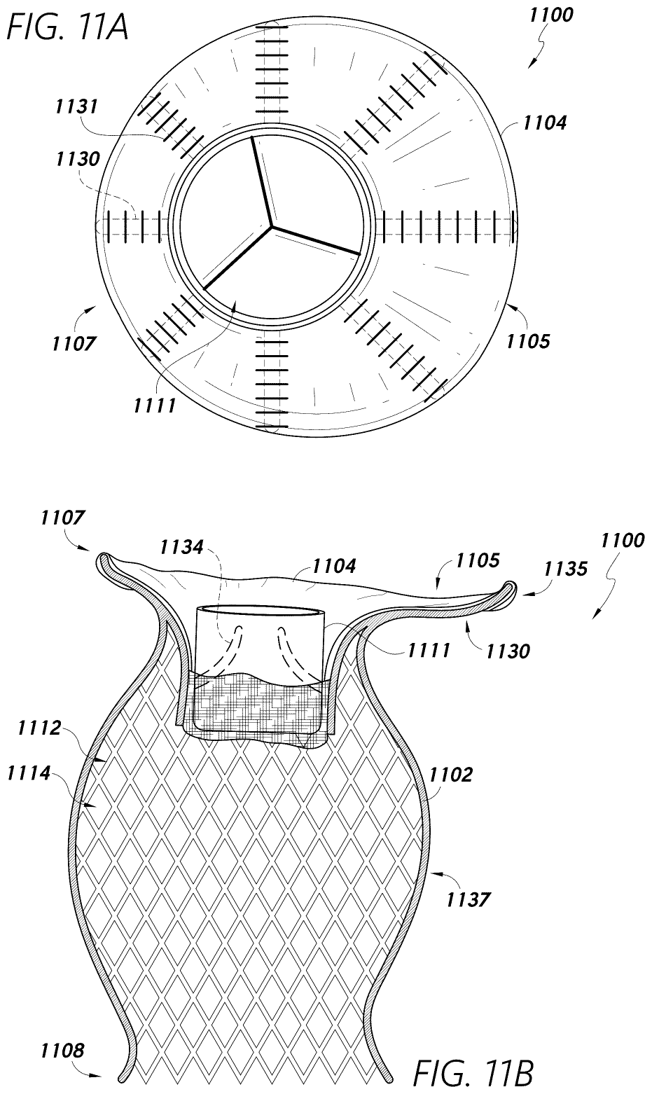

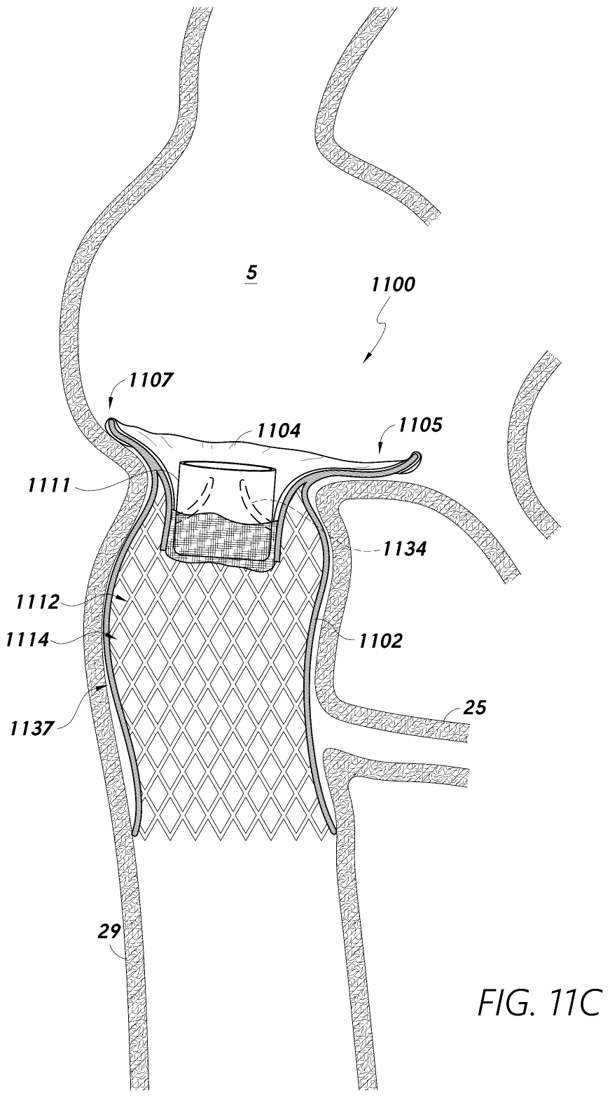

FIGS. 11A-11C illustrate an example stent 1100 and/or docking device (e.g., docking stent and/or docking station) configured for docking one or more prosthetic valves 1111 in accordance with one or more examples. FIG. 11A provides a top view of the stent 1100, FIG. 11B provides a side view of the stent 1100, and FIG. 11C illustrates the stent 1100 disposed at a junction between a right atrium 5 and an IVC 10. The stent 1100 may be configured to dock one or more prosthetic valves 1111, which can comprise one or more leaflets 1134.

Some example docking devices and/or stents can comprise one or more generally symmetrical and/or circular components. In some examples, one or more components can be generally concentrically aligned. However, patient anatomy (e.g., blood vessels, inflow junctions, and/or valves) can often have shapes that are not circular. As a result, the anatomy can be forced into compliance with a generally circular implant. This may have a variety of undesired consequences, which can include tissue erosion.

The stent 1100 can comprise a generally non-circular and/or non-concentric sealing element 1104. The sealing element 1104 may be disposed at a distal end 1107 of the stent 1100 and/or may form a distal flange of the stent 1100. In some examples, the sealing element 1104 can be non-concentric. In some examples, the sealing element 1104 can have a generally circular shape and/or may be at least partially offset from an inner lumen of the frame 1102 of the stent 1100. The stent 1100 may be configured for docking one or more prosthetic valves at the inner lumen of the frame 1102. The sealing element 1104 can have any suitable shape, which can include an elliptical, oval, and/or other curved and/or asymmetric geometry.

The frame 1102 can comprise an inner frame configured to grasp and/or dock the one or more prosthetic valves 1111 and/or an outer frame configured to anchor to the native anatomy. The inner frame and/or outer frame can join at the distal end 1107 of the frame 1102 to form the flared and/or flanged distal end 1107 of the frame 1102. The distal end 1107 can comprise one or more arms 1130 configured to flare upwardly (e.g., distally) and/or outwardly. In some examples, the one or more arms 1130 may be coupled to the sealing element 1104. For example, one or more sutures and/or stiches 1131 may be configured to form one or more attachments between the sealing element 1104 and the one or more arms 1130. The one or more arms 1130 may have variable lengths and/or may be relatively long at or near the protruding edge 1105. In some examples, the sealing element 1104 may be configured to loop around the one or more arms 1130 and/or to form one or more loops 1135 around the arms 1130.

The stent 1100 can be manufactured using any suitable devices and/or processes. In some examples, a generally rectangular sheet of material (e.g., metal) may be laser ablated to form a wire mesh (e.g., frame 1102). The wire mesh can be bent into a cylinder and/or formed into a generally concentric and/or circular shape. Alternatively, rather than using a rectangular sheet of material, one relatively long edge 1105 of a cutout can have a non-linear shape. For example, the edge 1105 shape can resemble an envelope flap. After laser ablation, the wire mesh may be formed into a generally cylinder shape. However, due to the non-linear edge 1105, the flange portion (e.g., sealing element 1104) can include an extended portion and/or can extend outwardly at one side to a greater extent than at other sides.

In some examples, one or more radiopaque markers and/or similar devices may be coupled to the stent 1100 and/or can be used to identify the orientation of the stent 1100 to facilitate implantation and/or orientation of the stent 1100. Accordingly, the sealing element 1104 of the stent 1100 can be oriented to reduce forced compliance of the native tissue. The scaling element 1104 and/or stent 1000 can be rotational and/or the shape of the sealing element 1104 can allow for rotational orientation to avoid blocking of various blood vessels (e.g., the coronary ostium) and/or to align the relatively short portions of the sealing element 1104 around the blood vessel wall(s).

The outer and/or inner cross-sectional outlines of the sealing element 1104 can be round, nearly round, oval, and/or other desirable shape. The inner and/or outer cross-sectional outlines of the sealing element 1104 can be at least partially offset and/or different from one another, resulting in at least one area or region (e.g., the protruding edge 1105) of the sealing element 1104 that is thick relative to another area and/or region of the scaling skirt. For example, the inner and outer cross-sectional shapes may be approximately circular and/or may be at least partially offset from being concentric, resulting in a portion of the sealing element 1104 being thicker. In another example, the inner cross-sectional shape may be circular while the outer shape is elliptical such that two thicker regions may be formed even though the two shapes may be concentric. The inner region of the sealing element 1104 may be attached to the wire mesh frame 1102 of the stent 1100.

The sealing element 1104 (e.g., sealing skirt) may facilitate an improved seal without causing deformation of the anatomical structure shape of the annulus and/or native tissue. Furthermore, the offset sealing element 1104 element may be useful in obtaining a good seal when the native valves exhibit calcification. The stent 1100 can be configured to be balloon-expandable and/or self-expanding. Additionally, the stent 1100 can be configured to be partially deployed to visualize the orientation of the protruding edge 1105, possibly through the use of radiopaque markers. When partially deployed, the stent 1100 can be manipulated to place the thickened area of the sealing element 1104 in the correct orientation prior to final deployment of the device.

The stent 1100 may be configured for delivery and/or placement at an SVC and/or IVC of a heart. For example, the stent 1100 may be configured for placement at or near an in-flow junction of the SVC and/or IVC to the right atrium of the heart. The stent 1100 may be configured for antegrade delivery (e.g., via the femoral vein) and/or retrograde delivery (e.g., via the subclavian and/or jugular vein).

The frame 1102 may be at least partially composed of one or more shape memory alloys (e.g., Nitinol) and/or may be laser-cut into a desired form. In some examples, the frame 1102 and/or sealing element 1104 may have a generally flared, mushroom, and/or funnel shape and/or form at or near the distal end 1107 of the stent 1100. For example, the distal end 1107 of the stent 1100 may be flared and/or conical in shape to extend outward into the right atrium and/or to form a grip onto a rim of the right atrium. In some examples, the sealing element 1104 may be configured to extend beyond the frame 1102 and/or may form an extension of the frame 1102 to extend further laterally and/or longitudinally relative to the frame 1102. The frame 1102 may further comprise a proximal end 1108 having a straight or flared form. In some examples, the frame 1102 may comprise a bulge 1137 having an increased diameter relative to other portions of the frame 1102 to facilitate anchoring of the stent 1100.

The stent 1100 may be configured to present minimal blockage of one or more hepatic veins and/or other branching blood vessels. For example, a heart may generally include one or more hepatic veins branching into the IVC at or near a junction between the IVC and the right atrium. In some examples, the sealing element 1104 may not extend along a complete length of the frame 1102. For example, the sealing element 1104 may extend from the distal end 1107 of the stent 1100 to a midsection and/or midpoint of the stent 1100. The stent 1100 may be configured such that, when implanted at the junction between the IVC and the right atrium, the sealing element 1104 may not extend over the one or more hepatic veins and/or branching blood vessels. In some examples, the frame 1102 may at least partially extend over the hepatic veins and/or branching blood vessels and/or may allow blood flow from the branching blood vessels through cells 1114 of the frame 1102. The frame 1102 can comprise a network of wires and/or struts 1112 forming the cells 1114 of the frame 1102.

In some examples, the stent 1100 may comprise one or more radiopaque markers configured to facilitate delivery and/or use of the stent 1100. For example, one or more markers may be disposed along the sealing element 1104 to assist with aligning and/or docking one or more prosthetic valves at the stent 1100 and/or sealing element 1104. In another example, the stent 1100 may comprise one or more markers at the frame 1102 and/or sealing element 1104 at or near the distal end 1107 to facilitate positioning of the stent 1100 and/or distal end 1107 at the atrium junction and/or other anatomy.

In some examples, the stent 1100 may be configured to cause native anatomy to conform to a size and/or shape of the stent 1100. For example, the frame 1102, scaling element 1104, and/or distal end 1107 of the stent 1100 may be configured to press outwardly against the native tissue to create a larger opening to support a prosthetic valve.

The frame 1102 may be coupled to the sealing element 1104 and/or may be configured to mate with the sealing element 1104. In some examples, one or more sutures and/or other attachment features (e.g., clips, hooks, notches, stitches, adhesives, etc.) may be used to form an attachment between the frame 1102 and the sealing element 1104.

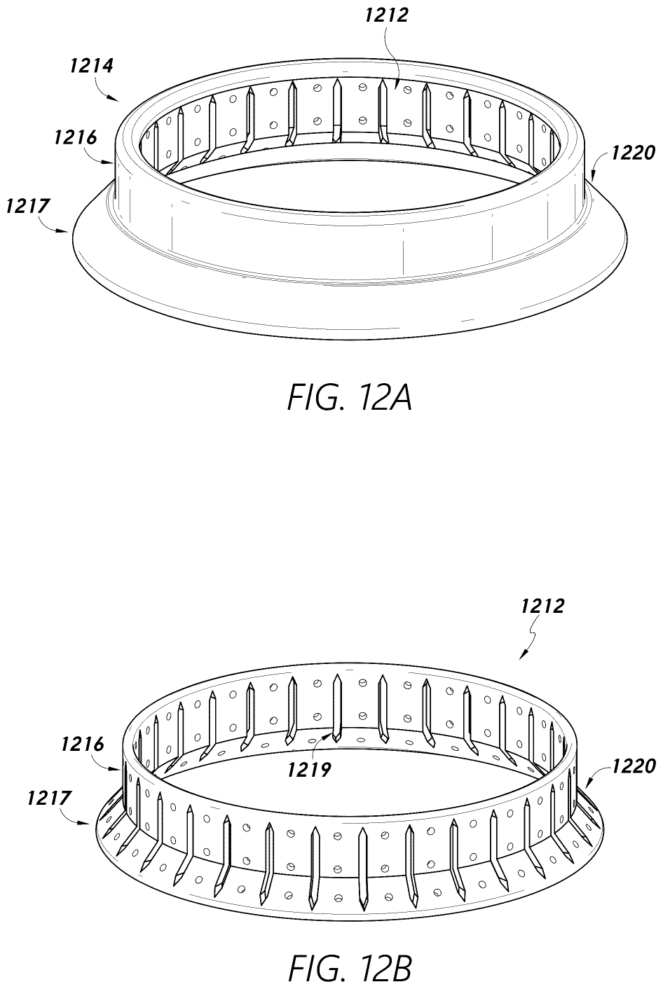

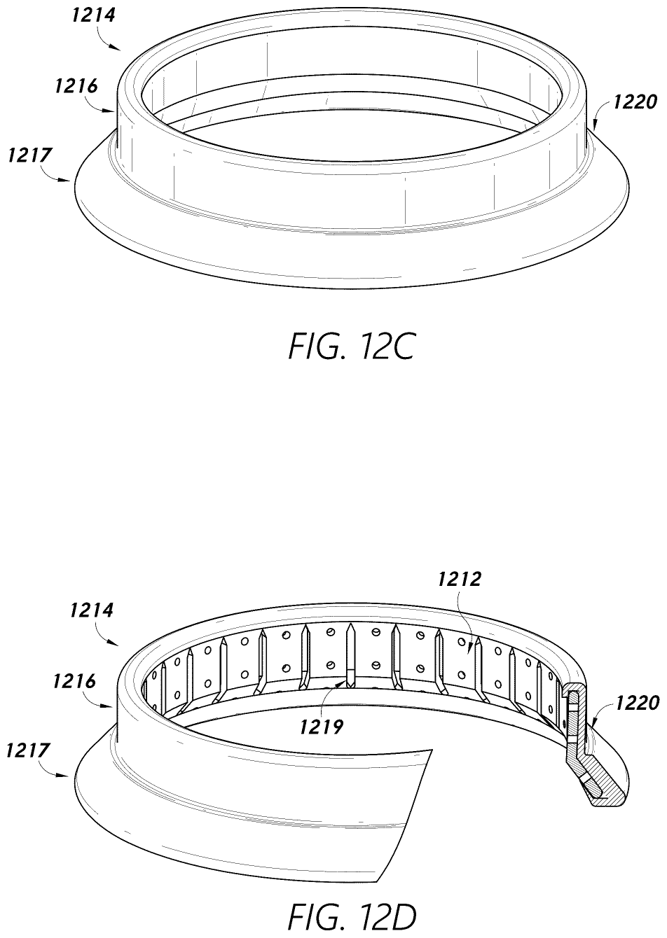

FIGS. 12A-12D illustrate components of an example sealing element 1214 for use with various stents and/or docking stations, including those described herein, in accordance with one or more examples. FIG. 12A illustrates an assembled sealing element 1214, FIG. 12B illustrates a frame 1212 of the sealing element 1214, FIG. 12C illustrates a skirt 1214 of the sealing element 1214, and FIG. 12D provides a cross-sectional view of the sealing element 1214.

Some prosthetic valves and/or docking stations can include a skirt configured to seal the area between the heart tissue and the prosthetic valves. However, in some instances leakage past the prosthetic valves can occur. The sealing element 1214 can be coupled to and/or used in combination with such prosthetic valves and/or stents to improve sealing of the prosthetic valves and/or stents.

The sealing element 1214 can comprise a ring-shaped sealing device having a metallic portion (e.g., frame 1212) and/or a sealing portion (e.g., skirt 1214). In some examples, the sealing element 1214 may be expandable. For example, the frame 1212 may be configured to compress prior to delivery to facilitate placement within a catheter and/or other delivery device. Upon delivery to a target location, the frame 1212 may be expanded to a desired size and/or shape to effectively form a seal between one or more prosthetic valves, stents, docking devices, and/or the anatomy. The skirt 1214 may have a generally soft and/or elastic form and/or may be configured to stretch and/or relax in response to movement of the frame 1212.

The sealing element 1214 may have a structure similar to a stent. For example, the frame 1212 may comprise wires, struts, cords, and/or similar components interconnected in a network and/or configured to expand to form cells between the various components and/or compress to close the cells.

The frame 1212 and/or skirt 1214 can be at least partially collapsible and/or may be generally cylindrical and/or ring shaped. A height of the frame 1212 and/or skirt 1214 may be less than a radius and/or diameter of the frame 1212 and/or skirt 1214. The frame 1212 and/or skirt 1214 can have a bent and/or flared distal end 1217 forming a flange and/or configured to facilitate anchoring and/or sealing of the sealing element 1214. The distal end 1217 may have a diameter that is greater than the diameters of other portions of the scaling element 1214. In some examples, the frame 1212 may comprise any suitable material(s) and/or may be at least partially composed of one or more shape memory alloys (e.g., Nitinol) and/or self-expanding material(s). The frame 1212 may be at least partially composed of stainless steel and/or various balloon-expandable material(s). In some examples, the frame 1212 may be generally solid and/or may comprise through slots 1219 and/or perforations. The frame 1212 may comprise one or more wire-like components.

The skirt 1214 may be composed of any suitable materials and/or may comprise PET, fabric, and/or other similar materials suitable for implantation and/or sealing. The skirt 1214 may be configured to at least partially cover an outer surface of the frame 1212, including the cylindrical body 1216 and/or flanged distal end 1217. In some examples, the skirt 1214 can wrap around proximal and/or distal ends of the frame 1212 and/or can extend at least partially over an inner surface of the frame 1212.

Delivery of the sealing element 1214 may be performed in any suitable method and/or process. In some examples, a stent and/or prosthetic valve may be first deployed into native anatomy at a target location within a patient's body. After the stent and/or valve is deployed, the sealing element 1214 can be positioned at a target location while in a compressed form before allowing self-expansion and/or balloon expansion of the scaling element 1214. Correct positioning of the sealing element 1214 may involve placing the sealing element 1214 such that the cylindrical portion 1216 of the sealing element 1214 is interior to the frame of the prosthetic valve and/or stent and/or such that a distal end of the prosthetic valve and/or stent is located at the bend point 1220 (e.g., juncture) between the cylinder 1216 and the flange 1217. Once properly positioned, the sealing element 1214 may be expanded and/or the skirt 1214 may be pressed against the anatomy to ensure the seal. Implantation of the sealing element 1214 can be performed immediately after implantation of the prosthetic valve and/or stent and/or at a time later if it is determined that leakage is occurring.

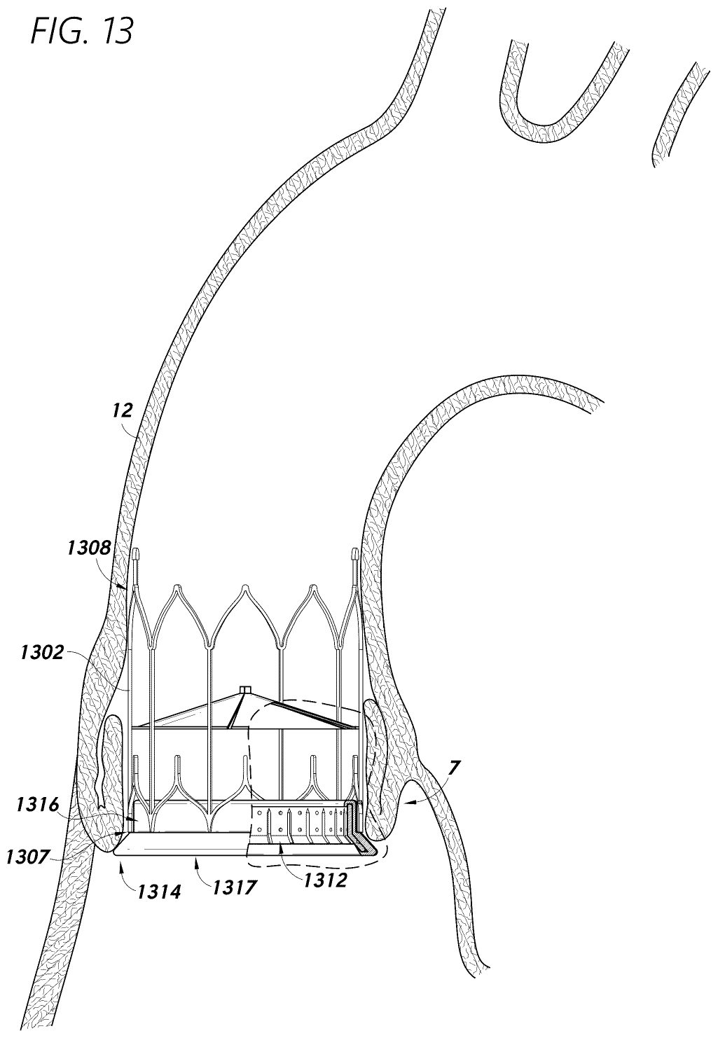

FIG. 13 illustrates an example stent comprising a stent body 1302 (e.g., frame) and a sealing element 1314, in accordance with one or more examples. The stent may be configured for delivery at various locations within a patient's body. In the example shown in FIG. 13, the stent is disposed at least partially within the aorta 12 and/or may be configured to provide a prosthetic aortic valve 7. However, the stent may be configured for delivery to other locations, including the IVC, SVC, and/or other blood vessels and/or chambers of the heart.

The stent is shown in an expanded form. At least some portions of the stent may be delivered in an at least partially compressed form. For example, the sealing element 1314 may comprise a frame 1312 configured to be expanded and/or the stent body 1302 may be configured to be expanded. The stent body 1302 and the sealing element 1314 may be separate components, may be coupled together, and/or may be extensions of a common device.

In some examples, the sealing element 1314 may be configured to expand at least partially beyond a blood vessel (e.g., the aorta 12) in which the stent body 1302 is disposed. For example, the sealing element 1314 may comprise a distal end 1317 configured to flare outwardly to form a lip and/or to grasp a rim of the valve and/or blood vessel.

The frame 1302 may comprise a proximal end 1308 configured to extend at least partially into the aorta 12 and/or other blood vessel. A distal end 1307 of the frame 1302 may be configured to be disposed at the valve 7 and/or at least partially beyond the aorta 12 and/or valve 7. In some examples, the distal end 1307 may be configured to be aligned with a bend point of the sealing element 1314. The sealing element 1314 may comprise a generally cylindrical portion 1316 configured to be disposed at least partially and/or fully within the frame 1302 of the stent and/or the flared and/or distal end 1317 of the sealing element 1314 may be configured to extend at least partially beyond the frame 1302 of the stent.

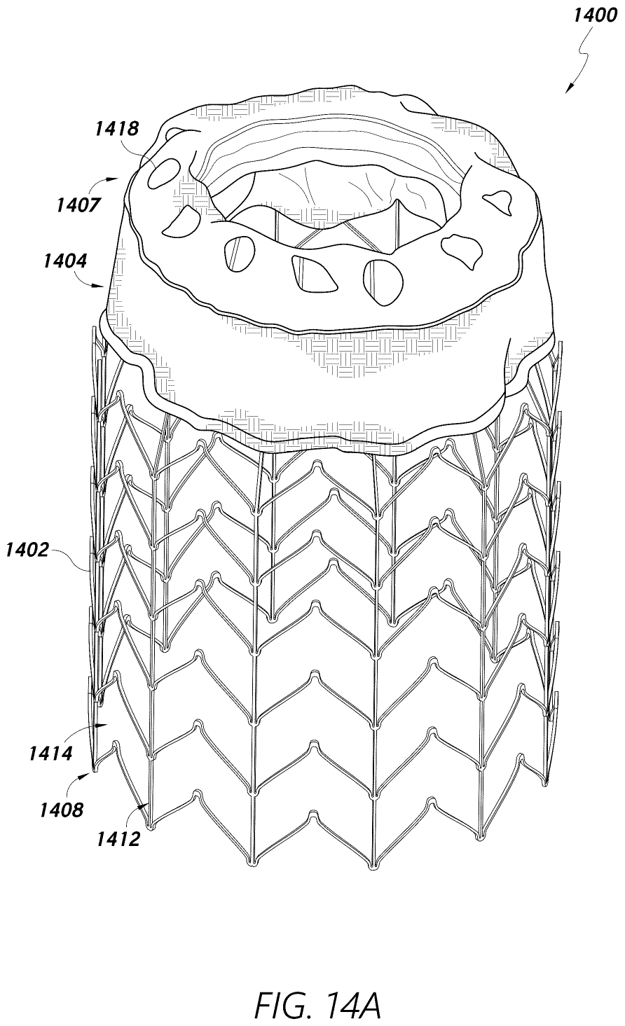

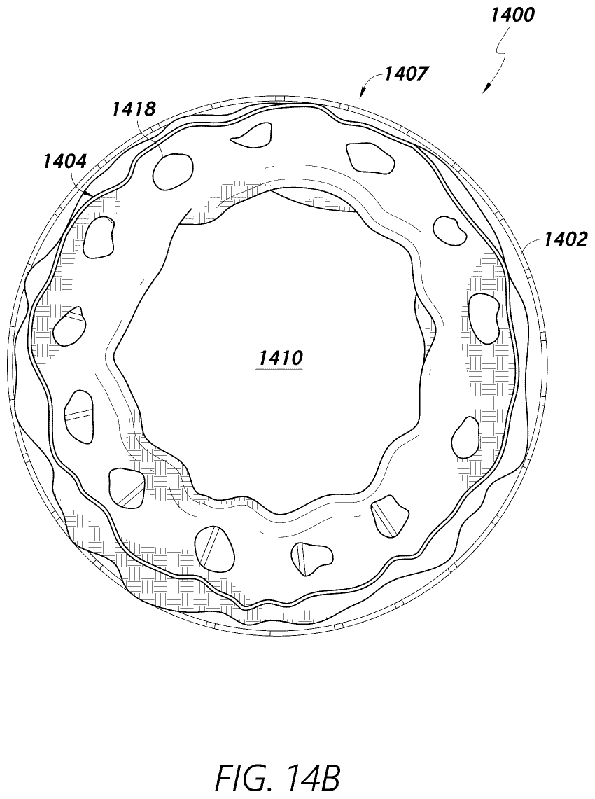

FIGS. 14A and 14B illustrate another example stent 1400 and/or docking station configured for placement withing one or more blood vessels and/or for docking one or more prosthetic valves in accordance with one or more examples. FIG. 14A provides a side view of the stent 1400 and FIG. 14B provides a top view of the stent 1400.

The stent 1400 can comprise a sealing element 1404 (e.g., scaling skirt) at or near a distal end 1407 and/or inlet side of the stent 1400. The stent 1400 can additionally comprise a frame 1402 configured to support and/or couple to the sealing element 1404. The frame 1402 may be at least partially composed of wires, cords, struts, and/or similar components and/or may be at least partially expandable and/or compressible. In some examples, the sealing element 1404 and/or frame 1402 may comprise an inner lumen 1410 configured to allow a portion of blood to flow through the sealing element 1404 and/or frame 1402. Blood flow through the inner lumen 1410 and/or apertures 1418 may cause inflation of the sealing element 1404 to improve sealing of the sealing element 1404 and/or stent 1400 against patient anatomy.

The sealing element 1404 may be stitched to the frame 1402 and/or implant framework along an inlet side of the stent 1400. The sealing element 1404 can cover at least a portion of an outer surface of the frame 1402 and/or can wrap at least partially around one or more ends (e.g., the distal end 1407) of the frame 1402 and/or can cover at least a portion of an inner surface of the frame 1402. The sealing element 1404 can be coupled to the frame 1402 by any suitable means. For examples, stitches near both ends of the sealing element 1404 can hold the sealing element 1404 in place on the frame 1402. The sealing element 1404 may be designed to provide a seal between the stent 1400 and/or frame 1402 and the patient's anatomy to prevent blood leakage past the stent 1400. The sealing element 1404 may be sufficiently long to create an adequate seal and/or sufficiently short to avoid blockage of the hepatic vein.

The sealing element 1404 may comprise one or more apertures 1418 (e.g., holes and/or openings) along one or more portions (e.g., the inlet edge) of the stent 1400. Blood flowing through the apertures 1418 can create pressure on the interior of the sealing element 1404, thereby pressing it against the anatomy and improving the sealing ability of the skirt. The apertures 1418 can have any suitable shape and/or size and/or the sealing element 1404 can have any number of apertures 1418. In the example shown in FIGS. 14A and 14B, the one or more apertures 1418 are aligned in series along a circumference of the sealing element 1404 and/or stent 1400. However, the one or more apertures 1418 can have any suitable alignment and/or position(s).

The stent 1400 may be configured for delivery and/or placement at an SVC and/or IVC of a heart. For example, the stent 1400 may be configured for placement at or near an in-flow junction of the SVC and/or IVC to the right atrium of the heart. The stent 1400 may be configured for antegrade delivery (e.g., via the femoral vein) and/or retrograde delivery (e.g., via the subclavian and/or jugular vein).

The frame 1402 may be at least partially composed of one or more shape memory alloys (e.g., Nitinol) and/or may be laser-cut into a desired form. In some examples, the frame 1402 and/or sealing element 1404 may have a generally flared, mushroom, and/or funnel shape and/or form at or near the distal end 1407 of the stent 1400. For example, the distal end 1407 of the stent 1400 may be flared and/or conical in shape to extend outward into the right atrium and/or to form a grip onto a rim of the right atrium. In some examples, the sealing element 1404 may be configured to extend beyond the frame 1402 and/or may form an extension of the frame 1402 to extend further laterally and/or longitudinally relative to the frame 1402. The frame 1402 may further comprise a proximal end 1408 having a straight or flared form.

The stent 1400 may be configured to present minimal blockage of one or more hepatic veins and/or other branching blood vessels. For example, a heart may generally include one or more hepatic veins branching into the IVC at or near a junction between the IVC and the right atrium. In some examples, the sealing element 1404 may not extend along a complete length of the frame 1402. For example, the sealing element 1404 may extend from the distal end 1407 of the stent 1400 to a midsection and/or midpoint of the stent 1400. The stent 1400 may be configured such that, when implanted at the junction between the IVC and the right atrium, the sealing element 1404 may not extend over the one or more hepatic veins and/or branching blood vessels. In some examples, the frame 1402 may at least partially extend over the hepatic veins and/or branching blood vessels and/or may allow blood flow from the branching blood vessels through cells 1414 of the frame 1402. The frame 1402 may comprise a network of wires and/or struts 1412 forming the cells 1414.

In some examples, the stent 1400 may comprise one or more radiopaque markers configured to facilitate delivery and/or use of the stent 1400. For example, one or more markers may be disposed along the sealing element 1404 to assist with aligning and/or docking one or more prosthetic valves at the stent 1400 and/or sealing element 1404. In another example, the stent 1400 may comprise one or more markers at the frame 1402 and/or scaling element 1404 at or near the distal end 1407 to facilitate positioning of the stent 1400 and/or distal end 1407 at the atrium junction and/or other anatomy.

In some examples, the stent 1400 may be configured to cause native anatomy to conform to a size and/or shape of the stent 1400. For example, the frame 1402, sealing element 1404, and/or distal end 1407 of the stent 1400 may be configured to press outwardly against the native tissue to create a larger opening to support a prosthetic valve. In some examples, the stent 1400 may comprise one or more bulbs configured to press outwardly against walls of a blood vessel and/or may be configured to correspondingly press walls of the blood vessel outwardly. The term “bulb” is used herein in accordance with its plain and ordinary meaning and may refer to any outward- and/or inward-extending portion of a frame 1402. In some examples, the frame 1402 may increase in diameter at the bulb and/or may decrease in diameter on either side of the bulb.

The frame 1402 may be coupled to the sealing element 1404 and/or may be configured to mate with the sealing element 1404. In some examples, one or more sutures and/or other attachment features (e.g., clips, hooks, notches, stitches, adhesives, etc.) may be used to form an attachment between the frame 1402 and the sealing element 1404.

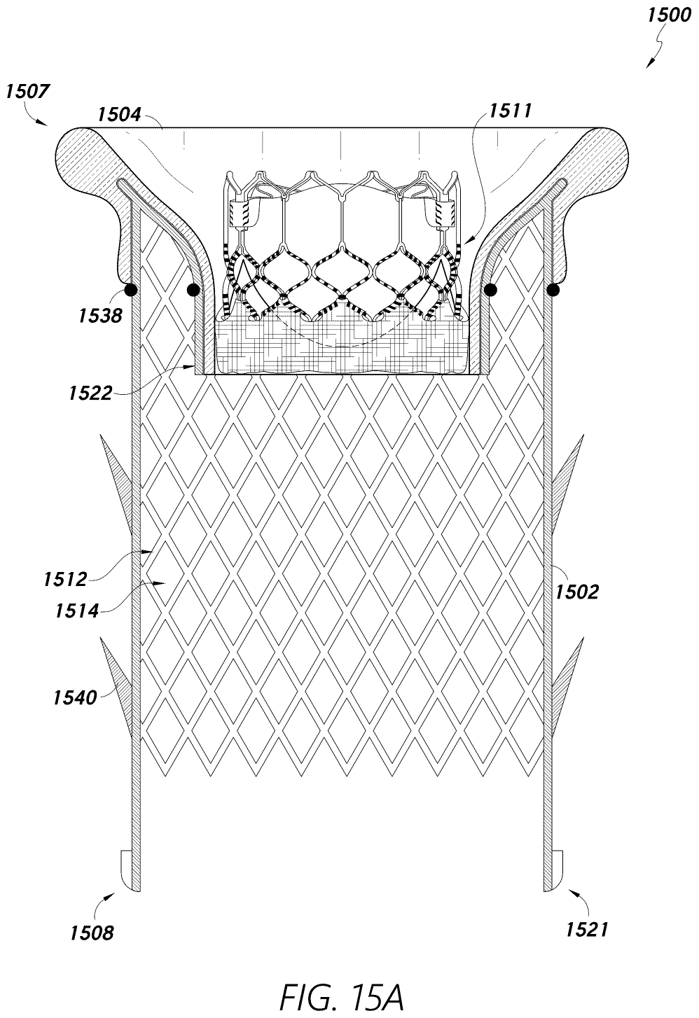

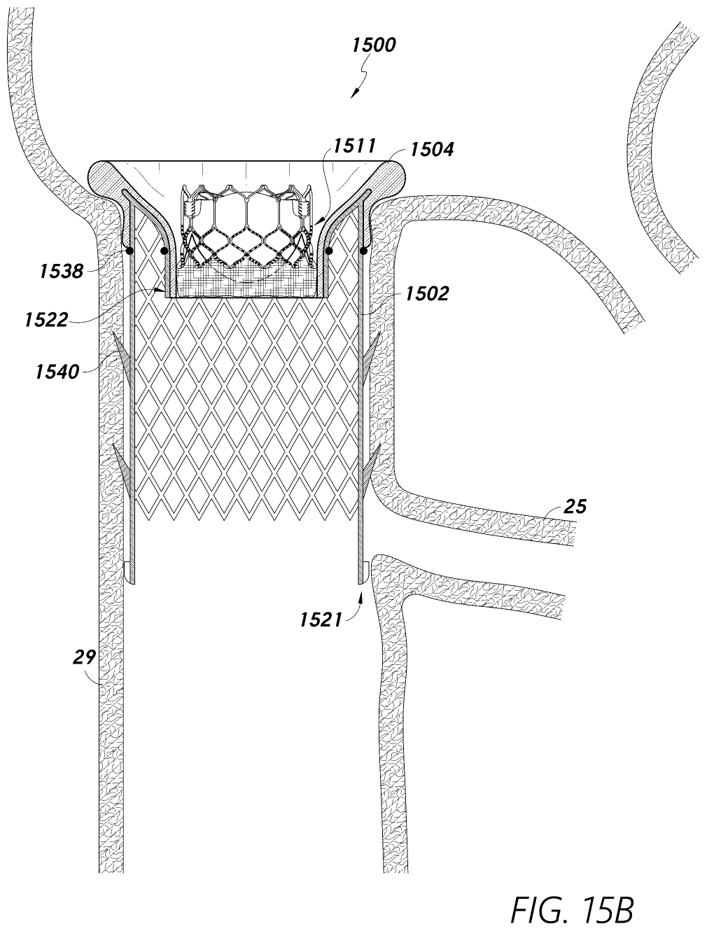

FIGS. 15A and 15B illustrate an example stent 1500 and/or docking device configured to be implanted at various blood vessels, valves, and/or other anatomies of a heart in accordance with one or more examples. FIG. 15A provides a side view of the stent 1500 and FIG. 15B provides a side view of the stent 1500 implanted at an inflow junction of the IVC 29 and the left atrium 5. However, the stent 1500 may be configured for delivery and/or deployment at other areas. The stent 1500 is shown with a prosthetic valve 1511 docked at least partially within an inner lumen of the stent 1500. However, the stent 1500 may be configured for delivery and/or use separate from the prosthetic valve 1511. In some examples, the frame 1502 may comprise an inner frame 1522 configured to facilitate docking of the prosthetic valve 1511.

The stent 1500 can comprise a frame 1502 and/or a sealing element 1504. The frame 1502 can be at least partially expandable and/or self-expanding. The sealing element 1504 may be composed of any suitable material(s), which can include PET and/or various fabrics. The sealing element 1504 can be disposed at or near a distal end 1507 of the frame 1502 and/or stent 1500. The sealing element 1504 can extend at least partially outwardly from an end of the stent 1500 and/or can form a generally soft flange to enhance sealing and/or prevent leakage.

The sealing element 1504 may be coupled to an outer surface of the frame 1502. In some examples, the stent 1500 may comprise coincident radiopaque markers 1538 along the sealing element 1504 to facilitate visualization of the sealing element 1504. The sealing element 1504 may be formed of PET fabric and/or similar material(s). The one or more radiopaque markers 1538 can serve to ensure that one or more branching blood vessels (e.g., hepatic veins) are not blocked by the sealing element 1504 and that the stent 1500 is properly positioned within the anatomy.

In some examples, the prosthetic valve 1511 can be prepositioned within the stent 1500 prior to crimping and insertion into a delivery system. The sealing element 1504 can form a seal between the frame 1502 and the implanted valve 1511.

The stent 1500 and/or frame 1502 can comprise one or more protruding anchors 1540 configured to facilitate anchoring of the stent 1500. For example, the one or more anchors 1540 can extend outwardly from the body of the frame 1502 and/or can comprise wire and/or struts bent outwardly. In some examples, the stent 1500 can comprise one or more proximal anchors 1521 configured to facilitate attachment of the frame 1502 to one or more delivery devices.

The stent 1500 may be configured for delivery and/or placement at an SVC and/or IVC of a heart. For example, the stent 1500 may be configured for placement at or near an in-flow junction of the SVC and/or IVC to the right atrium of the heart. The stent 1500 may be configured for antegrade delivery (e.g., via the femoral vein) and/or retrograde delivery (e.g., via the subclavian and/or jugular vein).

The frame 1502 may be at least partially composed of one or more shape memory alloys (e.g., Nitinol) and/or may be laser-cut into a desired form. In some examples, the frame 1502 and/or sealing element 1504 may have a generally flared, mushroom, and/or funnel shape and/or form at or near the distal end 1507 of the stent 1500. For example, the distal end 1507 of the stent 1500 may be flared and/or conical in shape to extend outward into the right atrium and/or to form a grip onto a rim of the right atrium. In some examples, the sealing element 1504 may be configured to extend beyond the frame 1502 and/or may form an extension of the frame 1502 to extend further laterally and/or longitudinally relative to the frame 1502. The frame 1502 may further comprise a proximal end 1508 having a straight or flared form.

The stent 1500 may be configured to present minimal blockage of one or more hepatic veins and/or other branching blood vessels. For example, a heart may generally include one or more hepatic veins branching into the IVC at or near a junction between the IVC and the right atrium. In some examples, the sealing element 1504 may not extend along a complete length of the frame 1502. For example, the sealing element 1504 may extend from the distal end 1507 of the stent 1500 to a midsection and/or midpoint of the stent 1500. The stent 1500 may be configured such that, when implanted at the junction between the IVC and the right atrium, the sealing element 1504 may not extend over the one or more hepatic veins and/or branching blood vessels. In some examples, the frame 1502 may at least partially extend over the hepatic veins and/or branching blood vessels and/or may allow blood flow from the branching blood vessels through cells 1514 of the frame 1502.

In some examples, the stent 1500 may comprise one or more radiopaque markers 1538 configured to facilitate delivery and/or use of the stent 1500. For example, one or more markers 1538 may be disposed along the sealing element 1504 to assist with aligning and/or docking one or more prosthetic valves at the stent 1500 and/or sealing element 1504. In another example, the stent 1500 may comprise one or more markers 1538 at the frame 1502 and/or scaling element 1504 at or near the distal end 1507 to facilitate positioning of the stent 1500 and/or distal end 1507 at the atrium junction and/or other anatomy.

In some examples, the stent 1500 may be configured to cause native anatomy to conform to a size and/or shape of the stent 1500. For example, the frame 1502, sealing element 1504, and/or distal end 1507 of the stent 1500 may be configured to press outwardly against the native tissue to create a larger opening to support a prosthetic valve. In some examples, the stent 1500 may comprise one or more bulbs configured to press outwardly against walls of a blood vessel and/or may be configured to correspondingly press walls of the blood vessel outwardly. The term “bulb” is used herein in accordance with its plain and ordinary meaning and may refer to any outward- and/or inward-extending portion of a frame 1502. In some examples, the frame 1502 may increase in diameter at the bulb and/or may decrease in diameter on either side of the bulb.

The frame 1502 may be coupled to the sealing element 1504 and/or may be configured to mate with the sealing element 1504. In some examples, one or more sutures and/or other attachment features (e.g., clips, hooks, notches, stitches, adhesives, etc.) may be used to form an attachment between the frame 1502 and the sealing element 1504.

The stent 1500 may comprise various components and/or features at the proximal end 1508 of the frame 1502. For example, the frame 1502 may comprise one or more edges formed by the wires 1512 of the frame 1502. The frame 1502 can comprise edges extending variable and/or different lengths. In some examples, the frame 1502 can comprise one or more anchors 1521 configured to facilitate attachment to one or more delivery devices (e.g., catheters), native anatomy, and/or other components of the stent 1500.

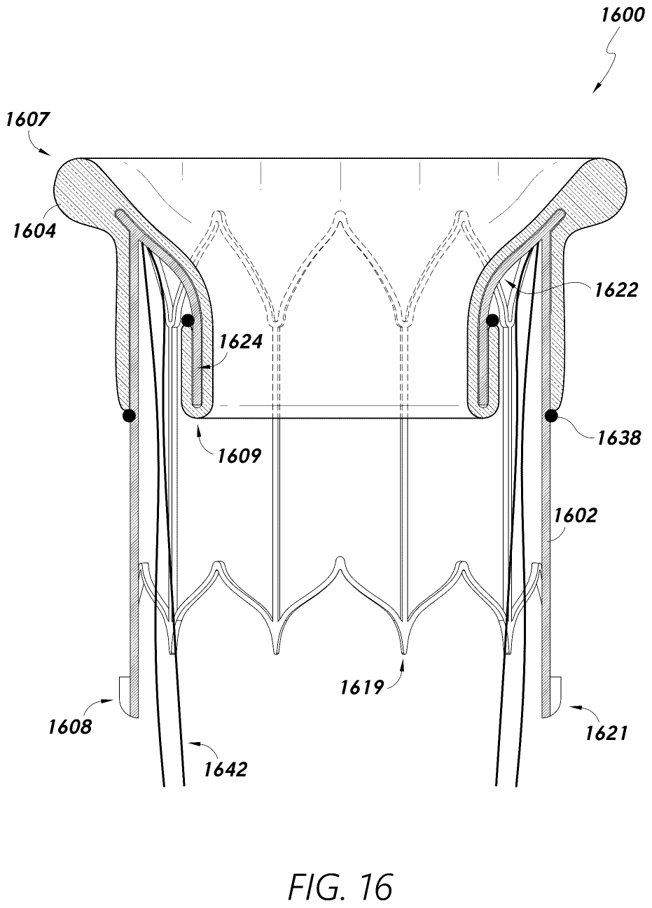

FIG. 16 illustrates another example stent 1600 and/or docking device configured to be implanted at various blood vessels, valves, and/or other anatomies of a heart in accordance with one or more examples. The stent 1600 can comprise a frame 1602 and/or sealing element 1604. In some examples, the sealing element 1604 can comprise a loop 1609 and/or bend to allow the sealing element 1604 to extend at least partially along an outer surface of the frame 1602 and/or along an inner surface of the frame 1602. in the skirt before it continues along the inside of the frame. The inner and/or outer portions of the sealing element 1604 can be attached to one or more ends of the frame 1602 to retain the loop 1609 at the end of the stent 1600. The loop 1609 in the sealing element 1604 can extend outwardly from the stent 1600 to form the sealing element 1604. The sealing element 1604 can continue along the inside of the frame 1602 (e.g., an inner frame 1622) to the end of a standoff 1624 located on the inner frame 1622. The sealing element 1604 can further wrap around the end of the standoff 1624 and/or can attach to the outer surface of the standoff 1624. In some examples, the sealing element 1604 can comprise one or more coincident radiopaque markers 1638 to facilitate placement of the sealing element 1604.

In some examples, the loop 1609 can be filled with one or more fillings. For example, a fuzzy fabric filler can be used inside the loop 1609 to further enhance the sealing ability of the sealing element 1604.

The stent 1600 may be configured for delivery and/or placement at an SVC and/or IVC of a heart. For example, the stent 1600 may be configured for placement at or near an in-flow junction of the SVC and/or IVC to the right atrium of the heart. The stent 1600 may be configured for antegrade delivery (e.g., via the femoral vein) and/or retrograde delivery (e.g., via the subclavian and/or jugular vein).

The frame 1602 may be at least partially composed of one or more shape memory alloys (e.g., Nitinol) and/or may be laser-cut into a desired form. In some examples, the frame 1602 and/or sealing element 1604 may have a generally flared, mushroom, and/or funnel shape and/or form at or near the distal end 1607 of the stent 1600. For example, the distal end 1607 of the stent 1600 may be flared and/or conical in shape to extend outward into the right atrium and/or to form a grip onto a rim of the right atrium. In some examples, the scaling element 1604 may be configured to extend beyond the frame 1602 and/or may form an extension of the frame 1602 to extend further laterally and/or longitudinally relative to the frame 1602. The frame 1602 may further comprise a proximal end 1608 having a straight or flared form.

The stent 1600 may be configured to present minimal blockage of one or more hepatic veins and/or other branching blood vessels. For example, a heart may generally include one or more hepatic veins branching into the IVC at or near a junction between the IVC and the right atrium. In some examples, the sealing element 1604 may not extend along a complete length of the frame 1602. For example, the sealing element 1604 may extend from the distal end 1607 of the stent 1600 to a midsection and/or midpoint of the stent 1600. The stent 1600 may be configured such that, when implanted at the junction between the IVC and the right atrium, the sealing element 1604 may not extend over the one or more hepatic veins and/or branching blood vessels. In some examples, the frame 1602 may at least partially extend over the hepatic veins and/or branching blood vessels and/or may allow blood flow from the branching blood vessels through cells of the frame 1602.

In some examples, the stent 1600 may comprise one or more radiopaque markers 1638 configured to facilitate delivery and/or use of the stent 1600. For example, one or more markers 1638 may be disposed along the sealing element 1604 to assist with aligning and/or docking one or more prosthetic valves at the stent 1600 and/or scaling element 1604. In another example, the stent 1600 may comprise one or more markers 1638 at the frame 1602 and/or scaling element 1604 at or near the distal end 1607 to facilitate positioning of the stent 1600 and/or distal end 1607 at the atrium junction and/or other anatomy.

In some examples, the stent 1600 may be configured to cause native anatomy to conform to a size and/or shape of the stent 1600. For example, the frame 1602, scaling element 1604, and/or distal end 1607 of the stent 1600 may be configured to press outwardly against the native tissue to create a larger opening to support a prosthetic valve. In some examples, the stent 1600 may comprise one or more bulbs configured to press outwardly against walls of a blood vessel and/or may be configured to correspondingly press walls of the blood vessel outwardly. The term “bulb” is used herein in accordance with its plain and ordinary meaning and may refer to any outward- and/or inward-extending portion of a frame 1602. In some examples, the frame 1602 may increase in diameter at the bulb and/or may decrease in diameter on either side of the bulb.

The frame 1602 may be coupled to the sealing element 1604 and/or may be configured to mate with the sealing element 1604. In some examples, one or more sutures and/or other attachment features (e.g., clips, hooks, notches, stitches, adhesives, etc.) may be used to form an attachment between the frame 1602 and the sealing element 1604.

The stent 1600 may comprise various components and/or features at the proximal end 1608 of the frame 1602. For example, the frame 1602 may comprise one or more edges 1619 formed by the wires and/or struts of the frame 1602. The frame 1602 can comprise edges 1619 extending variable and/or different lengths. In some examples, the frame 1602 can comprise one or more anchors 1621 configured to facilitate attachment to one or more delivery devices (e.g., catheters), native anatomy, and/or other components of the stent 1600.

In some examples, the sealing element 1604 can comprise one or more cords 1642 configured to facilitate movement of the sealing element 1604. For example, the one or more cords 1642 may comprise sutures configured to allow surgeons to selectively control loops 1609 formed by the sealing element 1604 and/or to release the loops 1609.

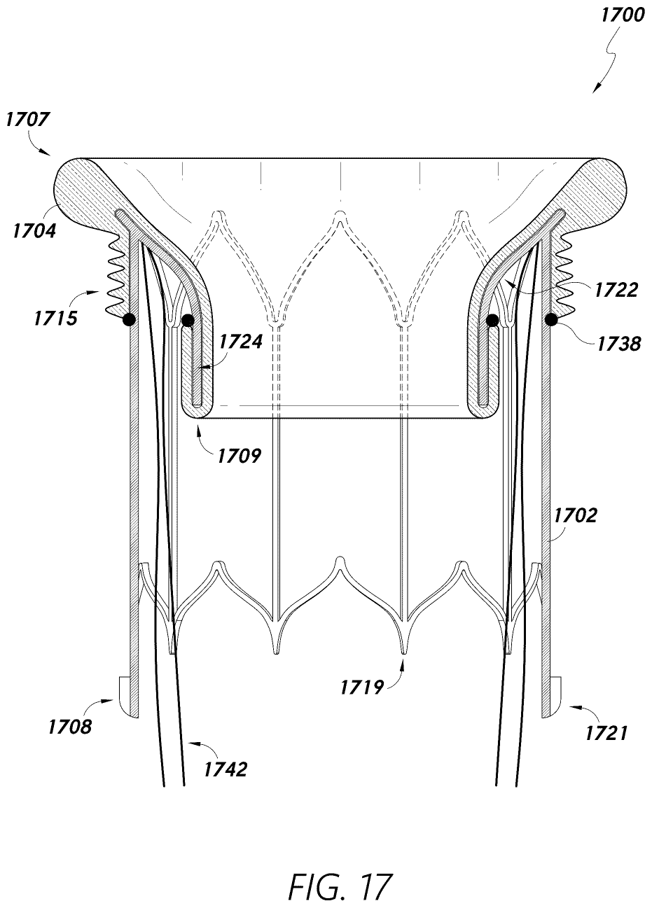

FIG. 17 illustrates another example stent 1700 and/or docking device configured to be implanted at various blood vessels, valves, and/or other anatomies of a heart in accordance with one or more examples. The stent 1700 can comprise a frame 1702 and/or scaling element 1704. In some examples, the sealing element 1704 can be at least partially elastic and/or stretchy. For example, at least a portion 1715 of the sealing element 1704 between an outer surface attachment point and an attachment point at the end of the frame 1702 can be made of an elastic fabric. By using elastic material, the fabric can stretch without breaking when the frame 1702 is crimped and/or can recover its original length when the stent 1700 is deployed.

The stent 1700 can comprise a frame 1702 and/or sealing element 1704. In some examples, the sealing element 1704 can comprise a loop 1709 and/or bend to allow the sealing element 1704 to extend at least partially along an outer surface of the frame 1702 and/or along an inner surface of the frame 1702. in the skirt before it continues along the inside of the frame. The inner and/or outer portions of the sealing element 1704 can be attached to one or more ends of the frame 1702 to retain the loop 1709 at the end of the stent 1700. The loop 1709 in the sealing element 1704 can extend outwardly from the stent 1700 to form the sealing element 1704. The sealing element 1704 can continue along the inside of the frame 1702 (e.g., an inner frame 1722) to the end of a standoff 1724 located on the inner frame 1722. The sealing element 1704 can further wrap around the end of the standoff 1724 and/or can attach to the outer surface of the standoff 1724. In some examples, the sealing element 1704 can comprise one or more coincident radiopaque markers 1738 to facilitate placement of the sealing element 1704.

In some examples, the loop 1709 can be filled with one or more fillings. For example, a fuzzy fabric filler can be used inside the loop 1709 to further enhance the scaling ability of the sealing element 1704.

The stent 1700 may be configured for delivery and/or placement at an SVC and/or IVC of a heart. For example, the stent 1700 may be configured for placement at or near an in-flow junction of the SVC and/or IVC to the right atrium of the heart. The stent 1700 may be configured for antegrade delivery (e.g., via the femoral vein) and/or retrograde delivery (e.g., via the subclavian and/or jugular vein).

The frame 1702 may be at least partially composed of one or more shape memory alloys (e.g., Nitinol) and/or may be laser-cut into a desired form. In some examples, the frame 1702 and/or sealing element 1704 may have a generally flared, mushroom, and/or funnel shape and/or form at or near the distal end 1707 of the stent 1700. For example, the distal end 1707 of the stent 1700 may be flared and/or conical in shape to extend outward into the right atrium and/or to form a grip onto a rim of the right atrium. In some examples, the sealing element 1704 may be configured to extend beyond the frame 1702 and/or may form an extension of the frame 1702 to extend further laterally and/or longitudinally relative to the frame 1702. The frame 1702 may further comprise a proximal end 1708 having a straight or flared form.

The stent 1700 may be configured to present minimal blockage of one or more hepatic veins and/or other branching blood vessels. For example, a heart may generally include one or more hepatic veins branching into the IVC at or near a junction between the IVC and the right atrium. In some examples, the sealing element 1704 may not extend along a complete length of the frame 1702. For example, the sealing element 1704 may extend from the distal end 1707 of the stent 1700 to a midsection and/or midpoint of the stent 1700. The stent 1700 may be configured such that, when implanted at the junction between the IVC and the right atrium, the sealing element 1704 may not extend over the one or more hepatic veins and/or branching blood vessels. In some examples, the frame 1702 may at least partially extend over the hepatic veins and/or branching blood vessels and/or may allow blood flow from the branching blood vessels through cells of the frame 1702.

In some examples, the stent 1700 may comprise one or more radiopaque markers 1738 configured to facilitate delivery and/or use of the stent 1700. For example, one or more markers 1738 may be disposed along the sealing element 1704 to assist with aligning and/or docking one or more prosthetic valves at the stent 1700 and/or sealing element 1704. In another example, the stent 1700 may comprise one or more markers 1738 at the frame 1702 and/or sealing element 1704 at or near the distal end 1707 to facilitate positioning of the stent 1700 and/or distal end 1707 at the atrium junction and/or other anatomy.

In some examples, the stent 1700 may be configured to cause native anatomy to conform to a size and/or shape of the stent 1700. For example, the frame 1702, scaling element 1704, and/or distal end 1707 of the stent 1700 may be configured to press outwardly against the native tissue to create a larger opening to support a prosthetic valve. In some examples, the stent 1700 may comprise one or more bulbs configured to press outwardly against walls of a blood vessel and/or may be configured to correspondingly press walls of the blood vessel outwardly. The term “bulb” is used herein in accordance with its plain and ordinary meaning and may refer to any outward- and/or inward-extending portion of a frame 1702. In some examples, the frame 1702 may increase in diameter at the bulb and/or may decrease in diameter on either side of the bulb.

The frame 1702 may be coupled to the sealing element 1704 and/or may be configured to mate with the sealing element 1704. In some examples, one or more sutures and/or other attachment features (e.g., clips, hooks, notches, stitches, adhesives, etc.) may be used to form an attachment between the frame 1702 and the sealing element 1704.

The stent 1700 may comprise various components and/or features at the proximal end 1708 of the frame 1702. For example, the frame 1702 may comprise one or more edges 1719 formed by the wires of the frame 1702. The frame 1702 can comprise edges 1719 extending variable and/or different lengths. In some examples, the frame 1702 can comprise one or more anchors 1721 configured to facilitate attachment to one or more delivery devices (e.g., catheters), native anatomy, and/or other components of the stent 1700.

In some examples, the sealing element 1704 can comprise one or more cords 1742 configured to facilitate movement of the sealing element 1704. For example, the one or more cords 1742 may comprise sutures configured to allow surgeons to selectively control loops 1709 formed by the sealing element 1704 and/or to release the loops 1709.

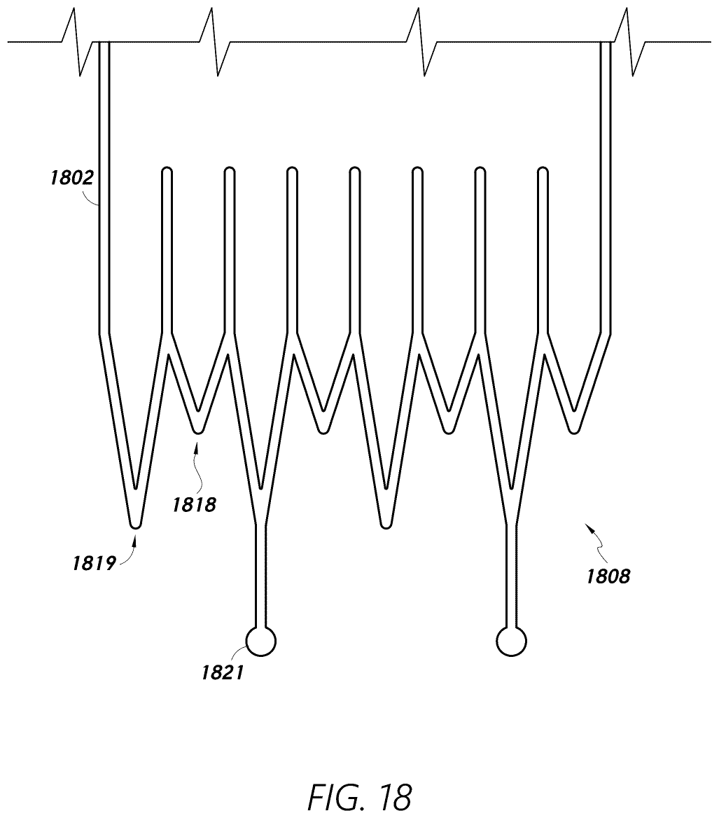

FIG. 18 illustrates an example flat pattern of an example frame 1802 of a stent and/or docking device in accordance with one or more examples. In some examples, the frame 1802 can include relatively long proximal edges 1819 and/or relatively short proximal edges 1818. The differing lengths of the cells can improve stability of the frame 1802 during deployment.

The stent may comprise various components and/or features at the proximal end 1808 of the frame 1802. For example, the frame 1802 may comprise one or more edges formed by the wires of the frame 1802. The frame 1802 can comprise edges extending variable and/or different lengths. In some examples, the frame 1802 can comprise one or more anchors 1821 configured to facilitate attachment to one or more delivery devices (e.g., catheters), native anatomy, and/or other components of the stent.

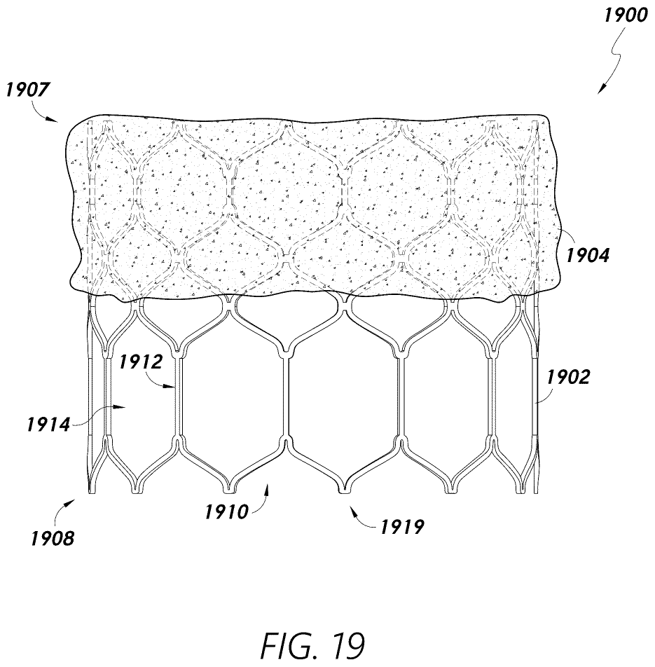

FIG. 19 illustrates an example stent 1900 and/or docking device configured for docking one or more prosthetic valves and comprising a distal sealing element 1904 at or near a distal end 1907 of the stent 1900 configured to increase surface friction at the distal end 1907 of the stent in accordance with one or more examples. In some examples, the sealing element 1904 can comprise a thick fabric and/or cloth configured to at least partially enclose the distal end 1907. The stent 1900 may further comprise a frame 1902 having a generally cylindrical and/or tubular form. In some examples, the frame 1902 may comprise a network of one or more wires 1912 (e.g., struts and/or cords) forming one or more cells 1914 between the wires 1912. The network of wires 1912 may have any suitable form generally diamond-shaped and/or hexagonal cells 1914. In some examples, cells 1914 at or near the distal end 1907 of the stent 1900 may have different forms than cells 1914 at or near the proximal end 1908 of the stent 1900.

The sealing element 1904 and/or cloth may have a generally thick structure and/or may increase a diameter of the frame 1902 and/or may extend from the frame 1902. The sealing element 1904 may be configured to improve sealing of the stent 1900 at the distal end 1907. In some examples, the sealing element 1904 may have a fibrous and/or fuzzy texture to increase friction between the stent 1900 and a blood vessel and/or blood vessel junction (e.g., at or near the RA/IVC junction).

While the stent 1900 is shown comprising a sealing element 1904 only at the distal end 1907 of the stent 1900, the stent 1900 may comprise multiple sealing elements 1904 and/or the sealing element 1904 may be configured to extend further along the frame 1902. In some examples, the sealing element 1904 may be disposed at an outer surface of the frame 1902. The sealing element 1904 may have a ring and/or generally circular shape and/or may be configured to extend along a full circumference of the frame 1902.

The sealing element 1904 may be configured to extend at least partially over a leading end of the frame 1902 and/or to extend onto an inner surface of the frame 1902. In some examples, the sealing element 1904 may be configured to increase friction and/or grip with a prosthetic valve placed into an inner lumen 1910 of the frame 1902. For example, the sealing element 1904 may increase a width of the stent 1900 and/or may be configured to extend from the inner surface of the frame 1902 to provide a contact point for one or more prosthetic valves placed into the frame 1902. The sealing element 1904 may be configured to pinch the prosthetic valve to securely hold the prosthetic valve.

The stent 1900 may be configured for delivery and/or placement at an SVC and/or IVC of a heart. For example, the stent 1900 may be configured for placement at or near an in-flow junction of the SVC and/or IVC to the right atrium of the heart. The stent 1900 may be configured for antegrade delivery (e.g., via the femoral vein) and/or retrograde delivery (e.g., via the subclavian and/or jugular vein).

The frame 1902 may be at least partially composed of one or more shape memory alloys (e.g., Nitinol) and/or may be laser-cut into a desired form. In some examples, the frame 1902 and/or sealing element 1904 may have a generally flared, mushroom, and/or funnel shape and/or form at or near the distal end 1907 of the stent 1900. For example, the distal end 1907 of the stent 1900 may be flared and/or conical in shape to extend outward into the right atrium and/or to form a grip onto a rim of the right atrium. In some examples, the sealing element 1904 may be configured to extend beyond the frame 1902 and/or may form an extension of the frame 1902 to extend further laterally and/or longitudinally relative to the frame 1902. The frame 1902 may further comprise a proximal end 1908 having a straight or flared form.

The stent 1900 may be configured to present minimal blockage of one or more hepatic veins and/or other branching blood vessels. For example, a heart may generally include one or more hepatic veins branching into the IVC at or near a junction between the IVC and the right atrium. In some examples, the sealing element 1904 may not extend along a complete length of the frame 1902. For example, the sealing element 1904 may extend from the distal end 1907 of the stent 1900 to a midsection and/or midpoint of the stent 1900. The stent 1900 may be configured such that, when implanted at the junction between the IVC and the right atrium, the sealing element 1904 may not extend over the one or more hepatic veins and/or branching blood vessels. In some examples, the frame 1902 may at least partially extend over the hepatic veins and/or branching blood vessels and/or may allow blood flow from the branching blood vessels through cells 1914 of the frame 1902.

In some examples, the stent 1900 may comprise one or more radiopaque markers configured to facilitate delivery and/or use of the stent 1900. For example, one or more markers may be disposed along the sealing element 1904 to assist with aligning and/or docking one or more prosthetic valves at the stent 1900 and/or sealing element 1904. In another example, the stent 1900 may comprise one or more markers at the frame 1902 and/or sealing element 1904 at or near the distal end 1907 to facilitate positioning of the stent 1900 and/or distal end 1907 at the atrium junction and/or other anatomy.

In some examples, the stent 1900 may be configured to cause native anatomy to conform to a size and/or shape of the stent 1900. For example, the frame 1902, sealing element 1904, and/or distal end 1907 of the stent 1900 may be configured to press outwardly against the native tissue to create a larger opening to support a prosthetic valve. In some examples, the stent 1900 may comprise one or more bulbs configured to press outwardly against walls of a blood vessel and/or may be configured to correspondingly press walls of the blood vessel outwardly. The term “bulb” is used herein in accordance with its plain and ordinary meaning and may refer to any outward- and/or inward-extending portion of a frame 1902. In some examples, the frame 1902 may increase in diameter at the bulb and/or may decrease in diameter on either side of the bulb.

The frame 1902 may be coupled to the sealing element 1904 and/or may be configured to mate with the sealing element 1904. In some examples, one or more sutures and/or other attachment features (e.g., clips, hooks, notches, stitches, adhesives, etc.) may be used to form an attachment between the frame 1902 and the sealing element 1904.

The stent 1900 may comprise various components and/or features at the proximal end 1908 of the frame 1902. For example, the frame 1902 may comprise one or more edges 1919 formed by the wires 1912 of the frame 1902. The frame 1902 can comprise edges 1919 extending variable and/or different lengths. In some examples, the frame 1902 can comprise one or more anchors configured to facilitate attachment to one or more delivery devices (e.g., catheters), native anatomy, and/or other components of the stent 1900.

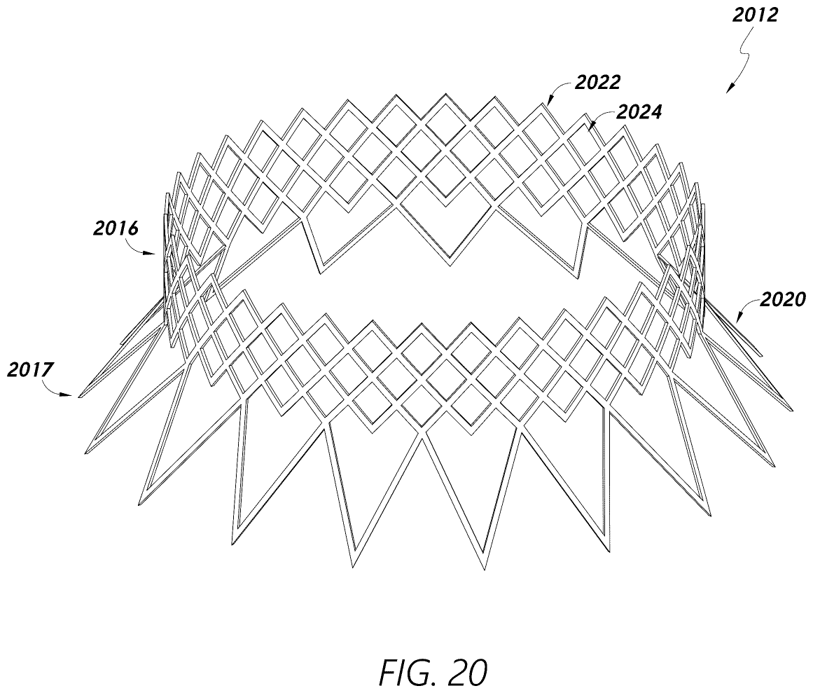

FIG. 20 illustrates a sealing element and/or a frame 2012 of a scaling element for use with various stents and/or implants described herein and/or otherwise. The frame 2012 can comprise any of a variety of suitable materials. For example, the frame 2012 may be at least partially composed of metal and/or various alloys. In some examples, the frame 2012 may be expandable. For example, the frame 2012 may be configured to compress prior to delivery to facilitate placement within a catheter and/or other delivery device. Upon delivery to a target location, the frame 2012 may be expanded to a desired size and/or shape to effectively form a seal between one or more prosthetic valves, stents, docking devices, and/or the anatomy. In some examples, the frame 2012 may be configured to be at least partially enclosed by a skirt and/or similar device having a generally soft and/or elastic form and/or configured to stretch and/or relax in response to movement of the frame 2012.

The frame 2012 may comprise a network of one or more wires 2022, struts, cords, and/or similar components interconnected in a network and/or configured to expand to form cells 2024 between the various components and/or compress to close the cells.

The frame 2012 can be at least partially collapsible and/or may be generally cylindrical and/or ring shaped. A height of the frame 2012 may be less than a radius and/or diameter of the frame 2012. The frame 2012 can have a bent and/or flared distal end 2017 forming a flange and/or configured to facilitate anchoring and/or sealing of a sealing element. The distal end 2017 may have a diameter that is greater than the diameters of other portions of the sealing element. In some examples, the frame 2012 may comprise any suitable material(s) and/or may be at least partially composed of one or more shape memory alloys (e.g., Nitinol) and/or self-expanding material(s). The frame 2012 may be at least partially composed of stainless steel and/or various balloon-expandable material(s). The frame 2012 may comprise one or more wires 2022 and/or wire-like components.

Delivery of the frame 2012 may be performed in any suitable method and/or process. In some examples, a stent and/or prosthetic valve may be first deployed into native anatomy at a target location within a patient's body. After the stent and/or valve is deployed, the frame 2012 can be positioned at a target location while in a compressed form before allowing self-expansion and/or balloon expansion of the frame 2012. Correct positioning of the frame 2012 may involve placing the frame 2012 such that the cylindrical portion 2016 of the frame 2012 is interior to the frame of the prosthetic valve and/or stent and/or such that a distal end of the prosthetic valve and/or stent is located at the bend point 2020 (e.g., juncture) between the cylinder 2016 and the flange 2017. Once properly positioned, the frame 2012 may be expanded and/or a skirt may be pressed against the anatomy to ensure the seal. Implantation of the frame 2012 can be performed immediately after implantation of the prosthetic valve and/or stent and/or at a time later if it is determined that leakage is occurring.

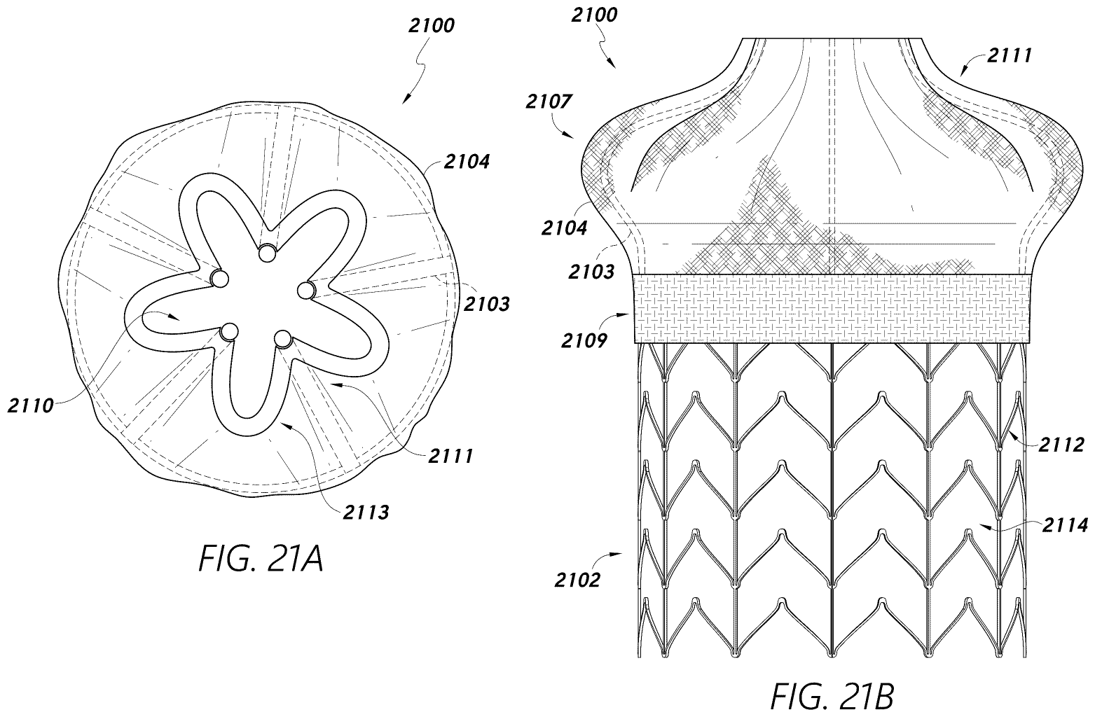

FIGS. 21A and 21B illustrate an implantable device 2100 with tissue covered and/or inwardly-biased distal arms 2103 configured to allow blood to flow in at least one direction and/or in only one direction in accordance with one or more examples. The one or more arms 2103 may be configured to form a valve (e.g., a one-way valve). In some examples, the one or more arms 2103 may be at least partially curved and/or may form a bulbous (e.g., bulb at a midsection), wavy, and/or hourglass shape. The one or more arms 2103 may be configured to at least partially straighten in response to pressure at the one or more arms 2103. For example, in response to increase blood flow through the device 2100, the one or more arms 2103 may be configured to at least partially straighten and/or otherwise assume an open form.

The device 2100 can comprise a self-expanding mesh frame 2102 and/or a sealing skirt 2104 at least partially enclosing the frame 2102. In some examples, the scaling skirt 2104 may at least partially enclose an exterior and/or interior surface of the frame 2102. The sealing skirt 2104 may be composed of any suitable material, which can include various tissues and/or polymers. The one or more (e.g., two or more) arms 2103 can extend from the frame 2102. In some examples, the arms 2103 can be disposed at a downstream and/or distal end of the frame 2102. The one or more arms 2103 can extend generally longitudinally from the frame 2102 and/or may not be interwoven and/or may not comprise the mesh form of a proximal portion of the frame 2102. The sealing skirt 2104 may be configured to at least partially enclose the one or more arms 2103. The sealing skirt 2104 may be composed of one or more suitable materials, which can include fabrics and/or polymers.

The device 2100 may comprise two or more arms 2103, which may be coupled to and/or may extend from the frame 2102. While the device 2100 is shown comprising five arms 2103, the device 2100 may comprise other numbers of arms 2103. In some examples, the 2103 may flare radially outwardly and/or may bend radially inwardly toward the cylindrical axis of the device 2100 to form a bulb 2107 and/or bulbous radial protrusion. The arms 2103 may be generally flexible and/or may be biased radially inwardly in a closed and/or default position. For example, the one or more arms 2103 may be biased to be pointed towards each other and/or towards a common point along a radial axis of the device 2100, as shown in FIG. 21A. The one or more arms 2103 may be configured to be flexed radially outwardly in an open position and/or in response to blood flow through the device 2100.

The frame 2102 may comprise a mesh of interwoven and/or interlocking struts 2112 forming cells 2114 between the struts 2112. A transition from a mesh region of the frame 2102 to the one or more arms 2103 may comprise a sealing zone 2109 which may comprise the sealing skirt 2104. For example, the sealing skirt 2104 may extend at least partially over the mesh of the frame 2102 and/or over the one or more arms 2103. The scaling skirt 2104 may be designed to provide a seal between the struts 2112 and/or arms 2103 and the patient's anatomy. Moreover, the sealing skirt 2104 may be configured to provide an attachment area for one or more prosthetic leaflets and/or may extend into one or more prosthetic leaflets 2111. In some examples, one or more radiopaque markers may be placed in the sealing zone 2109 to facilitate placement and/or alignment of the device 2100, thereby avoiding blockage of a hepatic vein and/or other flow pathway.

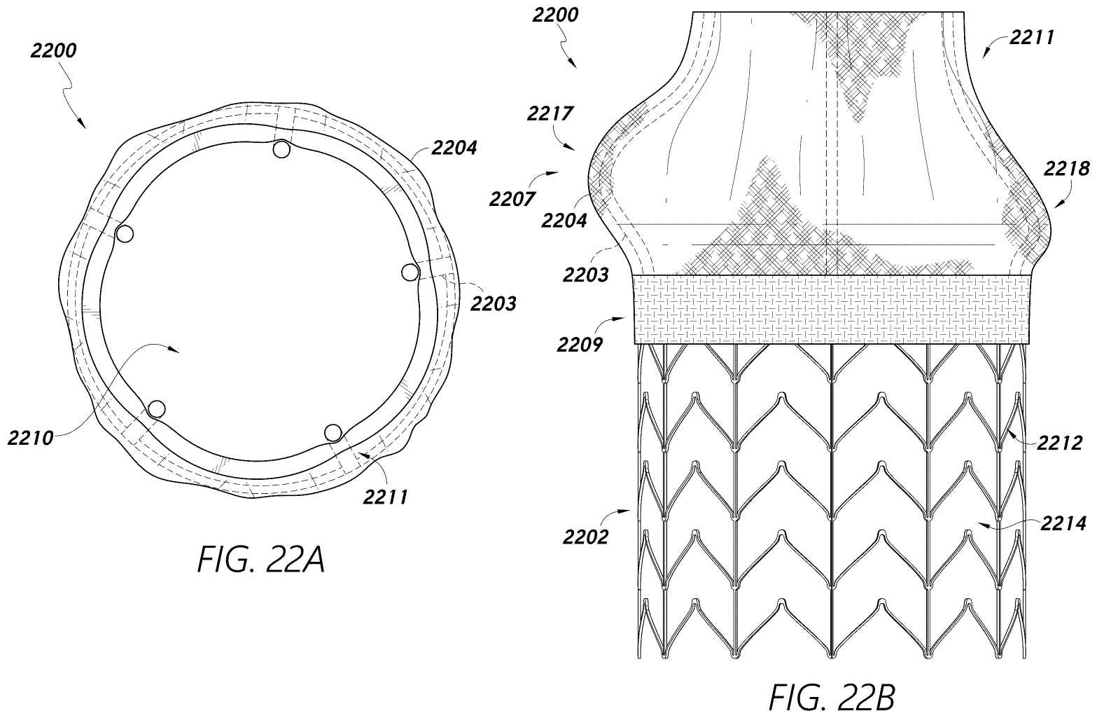

The leaflets 2111 may be attached to the skirt 2104 on one end and/or may extend distally to the distal ends of the arms 2103. The one or more leaflets 2111 may be disposed interior relative to the arms 2103 and/or may be disposed exterior to the arms 2103 to the arms 2103. The skirt 2104 and/or leaflets 2111 may comprise various materials and/or may be at least partially flexible. In some examples, the skirt 2104 and/or leaflets 2111 may comprise sufficient material to allow for radial expansion and/or straightening of the one or more arms 2103 (see, e.g., FIGS. 22A and 22B). When the device 2100 is in the open position illustrated in FIGS. 22A and 22B, the material of the leaflets 2111 may be relatively smooth and/or a clear opening and/or lumen through the device 2100 may be formed. When the device 2100 is in the closed position illustrated in FIGS. 21A and 21B, the excess material may be configured to form folds 2113 and/or leaflets to prevent backflow from the right atrium.

As blood flows from the inferior vena cava and/or superior vena cava into the right atrium, pressure from the veins may open and/or straighten the inwardly biased arms 2103, allowing blood to flow into the device 2100 and/or through the lumen 2110. As inward pressure subsides and the pressure in the right atrium increases, the arms 2103 may be configured to revert to the closed position shown in FIGS. 21A and 21B. Further increase in pressure from the right atrium side may push the arms 2103 closed even further, thereby preventing flow back into the inferior vena cava and/or superior vena cava.

The device may be configured for placement at least partially within an inferior vena cava, superior vena cava, right atrium, and/or other portions of anatomy. In some examples, the device 2100 can be adapted for use as a venous valve for use in a femoral vein and/or other vein and/or blood flow pathway. The device 2100 may be configured for use as a pulmonic valve replacement. The device 2100 may be configured to have any suitable length and/or diameter for a desired placement within a patient's body.

The device 2100 may comprise a stent and/or docking station configured for placement withing one or more blood vessels and/or for docking one or more prosthetic valves in accordance with one or more examples. The device 2100 can comprise a scaling element 2104 (e.g., sealing skirt) at or near a distal end and/or inlet side of the device 2100. The frame 2102 may be at least partially composed of wires, cords, struts, and/or similar components and/or may be at least partially expandable and/or compressible. In some examples, the sealing element 2104 and/or frame 2102 may comprise an inner lumen 2110 configured to allow a portion of blood to flow through the sealing element 2104 and/or frame 2102. Blood flow through the inner lumen 2110 and/or apertures may cause inflation of the sealing element 2104 to improve sealing of the sealing element 2104 and/or device 2100 against patient anatomy.

The sealing element 2104 may be stitched to the frame 2102 and/or implant framework along an inlet side of the device 2100. The sealing element 2104 can cover at least a portion of an outer surface of the frame 2102 and/or can wrap at least partially around one or more ends (e.g., the distal end) of the frame 2102 and/or can cover at least a portion of an inner surface of the frame 2102. The sealing element 2104 can be coupled to the frame 2102 by any suitable means. For examples, stitches near both ends of the sealing element 2104 can hold the sealing element 2104 in place on the frame 2102. The scaling element 2104 may be designed to provide a seal between the device 2100 and/or frame 2102 and the patient's anatomy to prevent blood leakage past the device 2100. The sealing element 2104 may be sufficiently long to create an adequate seal and/or sufficiently short to avoid blockage of the hepatic vein.

The device 2100 may be configured for delivery and/or placement at an SVC and/or IVC of a heart. For example, the device 2100 may be configured for placement at or near an in-flow junction of the SVC and/or IVC to the right atrium of the heart. The device 2100 may be configured for antegrade delivery (e.g., via the femoral vein) and/or retrograde delivery (e.g., via the subclavian and/or jugular vein).

The frame 2102 may be at least partially composed of one or more shape memory alloys (e.g., Nitinol) and/or may be laser-cut into a desired form. In some examples, the frame 2102 and/or sealing element 2104 may have a generally bulbous, flared, mushroom, and/or funnel shape and/or form at or near the distal end of the device 2100. For example, the distal end of the device 2100 may extend outwardly at or near a midsection of the device 2100 and/or may curve inwardly at or near the distal end of the device 2100. In some examples, the sealing element 2104 may be configured to extend beyond the frame 2102 and/or may form an extension of the frame 2102 to extend further laterally and/or longitudinally relative to the frame 2102. The frame 2102 may further comprise a proximal end having a straight or flared form.

The device 2100 may be configured to present minimal blockage of one or more hepatic veins and/or other branching blood vessels. For example, a heart may generally include one or more hepatic veins branching into the IVC at or near a junction between the IVC and the right atrium. In some examples, the sealing element 2104 may not extend along a complete length of the frame 2102. For example, the sealing element 2104 may extend from the distal end of the device 2100 to a midsection and/or midpoint of the device 2100. The device 2100 may be configured such that, when implanted at the junction between the IVC and the right atrium, the sealing element 2104 may not extend over the one or more hepatic veins and/or branching blood vessels. In some examples, the frame 2102 may at least partially extend over the hepatic veins and/or branching blood vessels and/or may allow blood flow from the branching blood vessels through cells 2114 of the frame 2102. The frame 2102 may comprise a network of wires and/or struts 2112 forming the cells 2114.

In some examples, the device 2100 may comprise one or more radiopaque markers configured to facilitate delivery and/or use of the device 2100. For example, one or more markers may be disposed along the sealing element 2104 to assist with aligning and/or docking one or more prosthetic valves at the device 2100 and/or sealing element 2104. In another example, the device 2100 may comprise one or more markers at the frame 2102 and/or sealing element 2104 at or near the distal end to facilitate positioning of the device 2100 and/or distal end at the atrium junction and/or other anatomy.

In some examples, the device 2100 may be configured to cause native anatomy to conform to a size and/or shape of the device 2100. For example, the frame 2102, sealing element 2104, and/or distal end of the device 2100 may be configured to press outwardly against the native tissue to create a larger opening to support a prosthetic valve. In some examples, the device 2100 may comprise one or more bulbs configured to press outwardly against walls of a blood vessel and/or may be configured to correspondingly press walls of the blood vessel outwardly. The term “bulb” is used herein in accordance with its plain and ordinary meaning and may refer to any outward- and/or inward-extending portion of a frame 2102. In some examples, the frame 2102 may increase in diameter at the bulb and/or may decrease in diameter on either side of the bulb.

The frame 2102 may be coupled to the sealing element 2104 and/or may be configured to mate with the sealing element 2104. In some examples, one or more sutures and/or other attachment features (e.g., clips, hooks, notches, stitches, adhesives, etc.) may be used to form an attachment between the frame 2102 and the sealing element 2104.

FIGS. 22A and 22B illustrate an implantable device 2200 with tissue covered and/or inwardly-biased distal arms 2203 configured to allow blood to flow in at least one direction and/or in only one direction in accordance with one or more examples. The one or more arms 2203 may be configured to form a valve (e.g., a one-way valve).

The device 2200 can comprise a self-expanding mesh frame 2202 and/or a sealing skirt 2204 at least partially enclosing the frame 2202. The sealing skirt 2204 may be composed of any suitable material, which can include various tissues and/or polymers. The one or more arms 2203 can extend from the frame 2202. In some examples, the arms 2203 can be disposed at a downstream and/or distal end of the frame 2202. The one or more arms 2203 can extend generally longitudinally from the frame 2202 and/or may not be interwoven and/or may not comprise the mesh form of a proximal portion of the frame 2202.

The device 2200 may comprise two or more arms 2203, which may be coupled to and/or may extend from the frame 2202. While the device 2200 is shown comprising five arms 2203, the device 2200 may comprise other numbers of arms 2203. In some examples, the 2203 may flare radially outwardly and/or may bend radially inwardly toward the cylindrical axis of the device 2200 to form a bulb 2207 and/or bulbous radial protrusion. The arms 2203 may be generally flexible and/or may be biased radially inwardly in a closed and/or default position. For example, the one or more arms 2203 may be biased to be pointed towards each other and/or towards a common point along a radial axis of the device 2200, as shown in FIG. 22A. The one or more arms 2203 may be configured to be flexed radially outwardly in an open position and/or in response to blood flow through the device 2200.

The frame 2202 may comprise a mesh of interwoven and/or interlocking struts 2212 forming cells 2214 between the struts 2212. A transition from a mesh region of the frame 2202 to the one or more arms 2203 may comprise a sealing zone 2209 which may comprise the sealing skirt 2204. For example, the sealing skirt 2204 may extend at least partially over the mesh of the frame 2202 and/or over the one or more arms 2203. The sealing skirt 2204 may be designed to provide a seal between the struts 2212 and/or arms 2203 and the patient's anatomy. Moreover, the sealing skirt 2204 may be configured to provide an attachment area for one or more prosthetic leaflets and/or may extend into one or more prosthetic leaflets 2211. In some examples, one or more radiopaque markers may be placed in the sealing zone 2209 to facilitate placement and/or alignment of the device 2200, thereby avoiding blockage of a hepatic vein and/or other flow pathway.

The leaflets 2211 may be attached to the skirt 2204 on one end and/or may extend distally to the distal ends of the arms 2203. The one or more leaflets 2211 may be disposed interior relative to the arms 2203 and/or may be disposed exterior to the arms 2203 to the arms 2203. The skirt 2204 and/or leaflets 2211 may comprise various materials and/or may be at least partially flexible. In some examples, the skirt 2204 and/or leaflets 2211 may comprise sufficient material to allow for radial expansion and/or straightening of the one or more arms 2203 (see, e.g., FIGS. 22A and 22B). When the device 2200 is in the open position illustrated in FIGS. 22A and 22B, the material of the leaflets 2211 may be relatively smooth and/or a clear opening and/or lumen through the device 2200 may be formed. When the device 2200 is in the closed position illustrated in FIGS. 21A and 21B, the excess material may be configured to form folds and/or leaflets to prevent backflow from the right atrium. The folds may be configured to be pulled taught and/or straightened in the open position of FIGS. 22A and 22B.

As blood flows from the inferior vena cava and/or superior vena cava into the right atrium, pressure from the veins may open and/or straighten the inwardly biased arms 2203, allowing blood to flow into the device 2200 and/or through the lumen 2210. As inward pressure subsides and the pressure in the right atrium increases, the arms 2203 may be configured to revert to the closed position shown in FIGS. 22A and 22B. Further increase in pressure from the right atrium side may push the arms 2203 closed even further, thereby preventing flow back into the inferior vena cava and/or superior vena cava.

The device may be configured for placement at least partially within an inferior vena cava, superior vena cava, right atrium, and/or other portions of anatomy. In some examples, the device 2200 can be adapted for use as a venous valve for use in a femoral vein and/or other vein and/or blood flow pathway. The device 2200 may be configured for use as a pulmonic valve replacement. The device 2200 may be configured to have any suitable length and/or diameter for a desired placement within a patient's body.

In some examples, the device 2200 may have an at least partially asymmetrical form. For example, the various arms 2203 of the device 2200 may have different shape-set forms and/or shapes. In some examples, the two or more arms 2203 may be shape-set such that a first side 2217 of the bulb 2207 extends further out radially than a second side 2218 of the bulb 2207. Moreover, the second side 2218 may be seated lower relative to the scaling zone 2209 than the first side 2217.

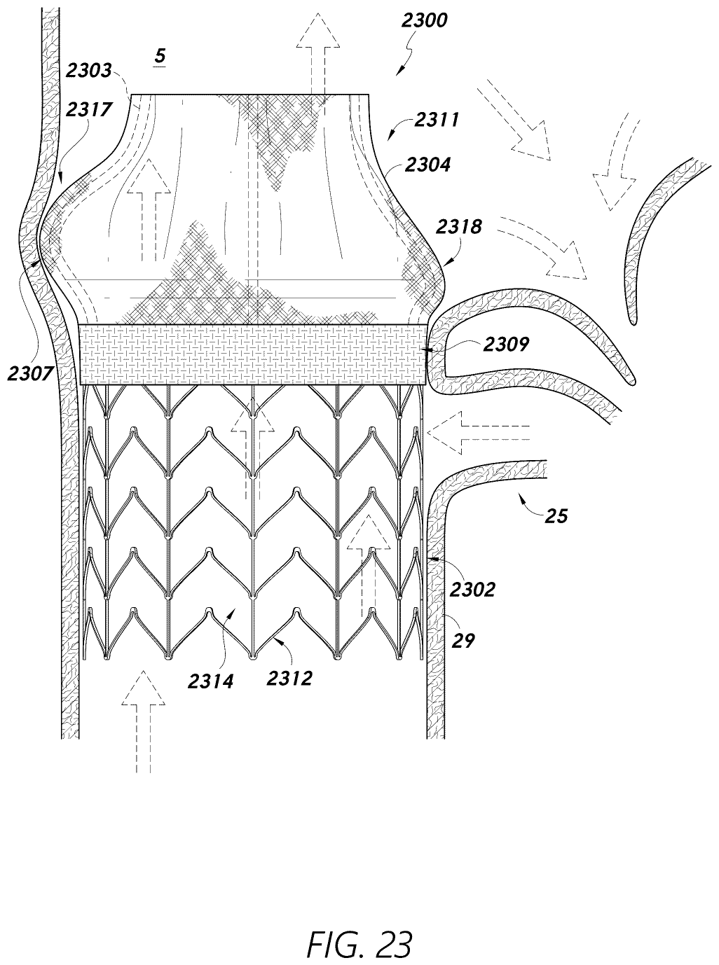

FIG. 23 illustrates an example device 2300 implanted at a junction between an inferior vena cava 29 and a right atrium 5 of a heart in accordance with one or more examples. The device 2300 may comprise one or more tissue covered and/or inwardly-biased distal arms 2303 configured to allow blood to flow in at least one direction and/or in only one direction in accordance with one or more examples. The one or more arms 2303 may be configured to form a valve (e.g., a one-way valve).

The device 2300 may be configured to present minimal blockage of one or more hepatic veins 25 and/or other branching blood vessels. For example, a heart may generally include one or more hepatic veins 25 branching into the IVC 29 at or near a junction between the IVC 29 and the right atrium 5. In some examples, the sealing element 2304 may not extend along a complete length of the frame 2302. For example, the scaling element 2304 may extend from the distal end of the device 2300 to a midsection and/or midpoint of the device 2300. The device 2300 may be configured such that, when implanted at the junction between the IVC 29 and the right atrium 5, the sealing element 2304 may not extend over the one or more hepatic veins 25 and/or branching blood vessels. In some examples, the frame 2302 may at least partially extend over the hepatic veins 25 and/or branching blood vessels and/or may allow blood flow from the branching blood vessels through cells 2314 of the frame 2302. The frame 2302 may comprise a network of wires and/or struts 2312 forming the cells 2314. The sealing element 2304 may comprise a sealing zone 2309 and/or sealing strip configured to securely attach to the frame 2302 and/or arms 2303. For example, the sealing zone 2309 may comprise an elasticity and/or may be configured to compress over the frame 2302 to maintain an attachment to the frame 2302. The skirt 2304 may be configured to form one or more leaflets 2311 extending at least partially across an inner lumen of the device 2300 and/or frame 2302.

In some examples, the device 2300 may have an at least partially asymmetrical form. For example, the various arms 2303 of the device 2300 may have different shape-set forms and/or shapes. In some examples, the two or more arms 2303 may be shape-set such that a first side 2317 of the bulb 2307 extends further out radially than a second side 2318 of the bulb 2307. Moreover, the second side 2318 may be seated lower relative to the sealing zone 2309 than the first side 2317.

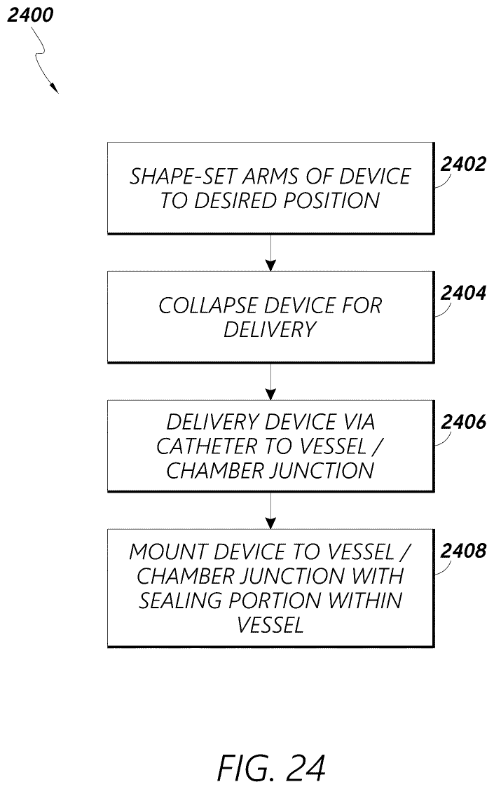

FIG. 24 provides a flowchart illustrating an example process 2400 for delivering various blood flow devices described herein, including the devices described in FIGS. 21-23.

At step 2402, the process 2400 involves shape-setting one or more arms of the device to a desired position. In some examples, the one or more arms may be shape-set to a wavy and/or bulbous form. The one or more arms may extend from an end of a generally cylindrical frame. In some examples, the one or more arms may extend radially outwardly relative to the frame and/or radially inwardly relative to the frame to form a bulbous shape.

At step 2404, the process 2400 involves collapsing and/or compressing the device for delivery via one or more catheters and/or similar devices. For example, the device may comprise a generally self-expanding network of struts and/or arms configured to elastically bend and/or compress in response to pressure.

At step 2406, the process 2400 involves delivering the device via a catheter to a blood vessel and/or chamber, and/or to a junction between a blood vessel and heart chamber (e.g., at the IVC/RA junction). The device may be at least partially disposed within the blood vessel and/or may extend at least partially into the chamber. For example, a bulbous portion of the device may extend into the chamber.

At step 2408, the process 2400 involves mounting the device to the junction of the blood vessel and chamber with a sealing portion of the device within the blood vessel. For example, the device may comprise a sealing skirt configured to at least partially extend along a cylindrical frame of the device. The sealing skirt may comprise a scaling zone configured to be disposed fully within the blood vessel.

The IVC/RA junction can include pouches, branching blood vessel (e.g., hepatic vein) entry points, and/or other anatomical variations that can create difficult surfaces against which to seal implants, including docking stations.

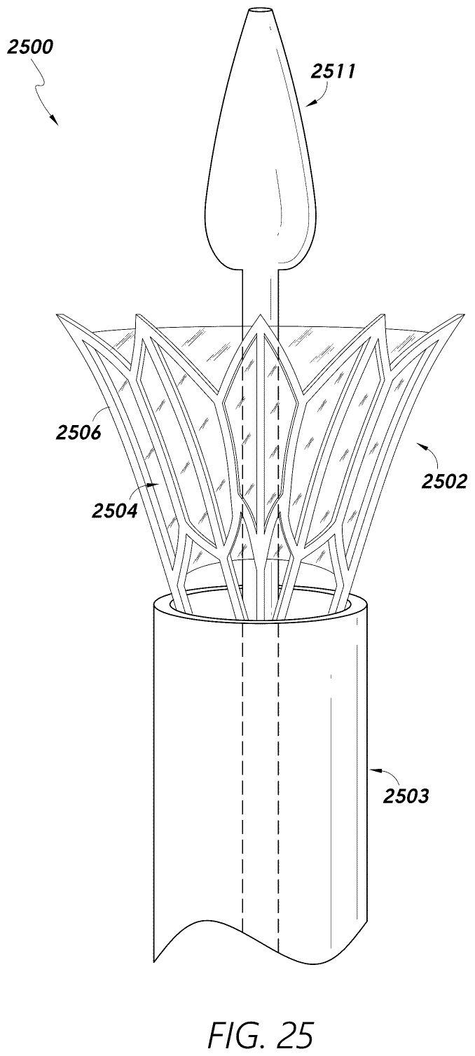

FIG. 25 illustrates an example delivery system 2500 for delivering an implant 2502 (e.g., stent and/or docking station) configured to form a seal with surrounding anatomy, in accordance with one or more examples. In some examples, the implant 2502 may be delivered via a catheter 2503 and/or other delivery device. A nose cone 2511 may be used as a leading edge of the delivery system 2500 and/or may be disposed within the catheter 2503 distally of the implant 2502 during delivery.

The implant 2502 may comprise one or more balloons 2504 exterior and/or interior to the implant 2502 and/or configured to form and/or facilitate a seal between the implant 2502 and the surrounding anatomy. The one or more balloons 2504 may be configured to inflate to seal against irregularities in the patient's anatomy and/or prevent leakage.

The implant 2502 can be configured for docking one or more prosthetic implants and/or may be configured for other/different uses. In some examples, the implant 2502 may comprise a frame 2506 composed of Nitinol, cobalt chromium, and/or other materials which may be configured to be balloon expanded from the interior and/or exterior of the implant 2502 to a predetermined size and/or to a size of a blood vessel in which the implant may be deployed.

The implant 2502 may comprise an interior and/or exterior balloon 2504 and/or can include a sealing skirt disposed at and/or around the interior and/or exterior balloon 2504. Inflation of an exterior balloon 2504 can create contact with the surrounding anatomy and/or the implant 2502, thereby forming a seal to prevent leakage. Inflation of an interior balloon 2504 can press the frame 2506 against the surrounding anatomy to facilitate scaling of the frame 2506 against the anatomy. The balloon 2504 can be configured to conform to various deviations in the anatomy that otherwise would be difficult to seal against. The balloon 2504 may be further configured to remain with the frame 2506 to maintain the seal following removal of the catheter 2503, nose cone 2511, and/or other delivery devices.

In some examples, the frame 2506 may be self-expandable. For example, upon removal from the catheter 2503, the frame 2506 may be configured to expand to a first expanded state. Inflation of the balloon 2504 may cause further expansion of the frame 2506.

The balloon 2504 may be configured to extend along only a portion of the frame 2506 and/or sealing skirt. For example, the balloon 2504 may be disposed at or near a proximal end of the frame 2506 (e.g., at or near an RA/IVC junction).

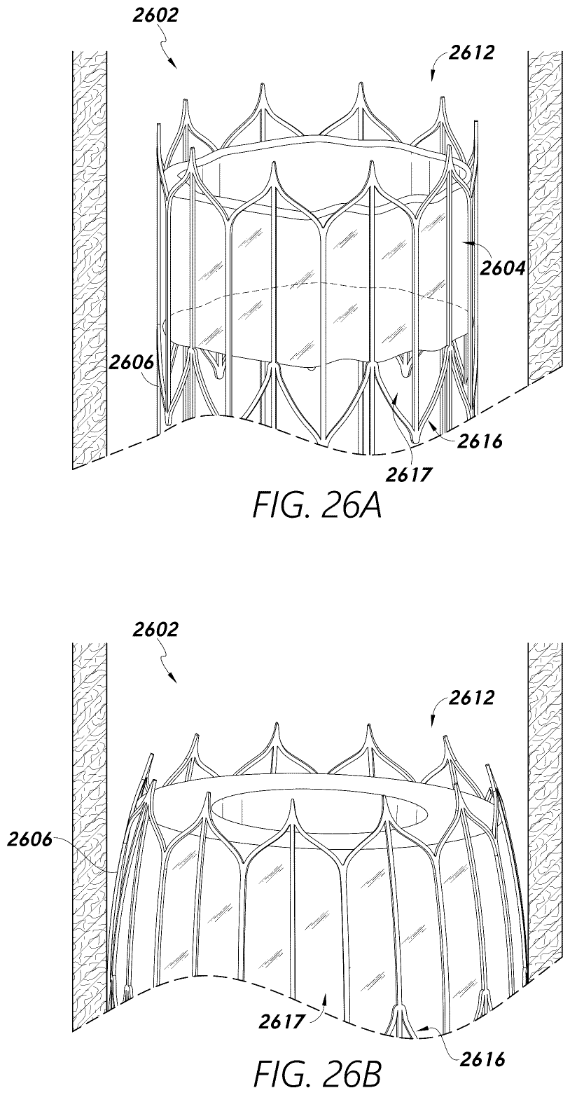

FIGS. 26A-26B illustrate an example implant 2602 comprising an interior balloon 2604 configured to form and/or facilitate a seal between a frame 2606 of the implant 2602 and the surrounding anatomy, in accordance with one or more examples. The implant 2602 is shown deployed within a blood vessel (e.g., the IVC). FIG. 26A illustrates the implant 2602 with the balloon 2604 in a deflated state. The frame 2606 may have a first expanded form in the deflated state. FIG. 26B illustrates the implant 2602 with the balloon 2604 in an inflated state. The frame 2606 may have a second expanded form in the inflated state in which a width and/or diameter of the frame 2606 exceeds the width and/or diameter in the first expanded form. The interior balloon 2604 may be configured to inflate to press the frame 2606 against irregularities in the patient's anatomy and/or prevent leakage. However, inflation of the balloon 2604 may not necessarily press the frame 2606 into contact with the surrounding anatomy. For example, gaps may exist between the frame 2606 and the walls of the blood vessel.

The implant 2602 can be configured for docking one or more prosthetic implants and/or may be configured for other/different uses. In some examples, the implant 2602 may comprise a frame 2606 composed of Nitinol, cobalt chromium, and/or other materials which may be configured to be balloon expanded from the interior and/or exterior of the implant 2602 to a predetermined size and/or to a size of a blood vessel in which the implant may be deployed.

The implant 2602 may comprise an interior balloon 2604 and can include a scaling skirt disposed at and/or around the balloon 2604. The balloon 2604 can be configured to conform to various deviations in the anatomy that otherwise would be difficult to seal against. The balloon 2604 may be further configured to remain with the frame 2606 to maintain the seal.

In some examples, the frame 2606 may be self-expandable. The balloon 2604 may be configured to extend along only a portion of the frame 2606 and/or sealing skirt. In some examples, a prosthetic valve may be docked at least partially within the frame 2606 and/or balloon 2604. For example, the balloon 2604 may provide a platform and/or base for placing one or more prosthetic valves.

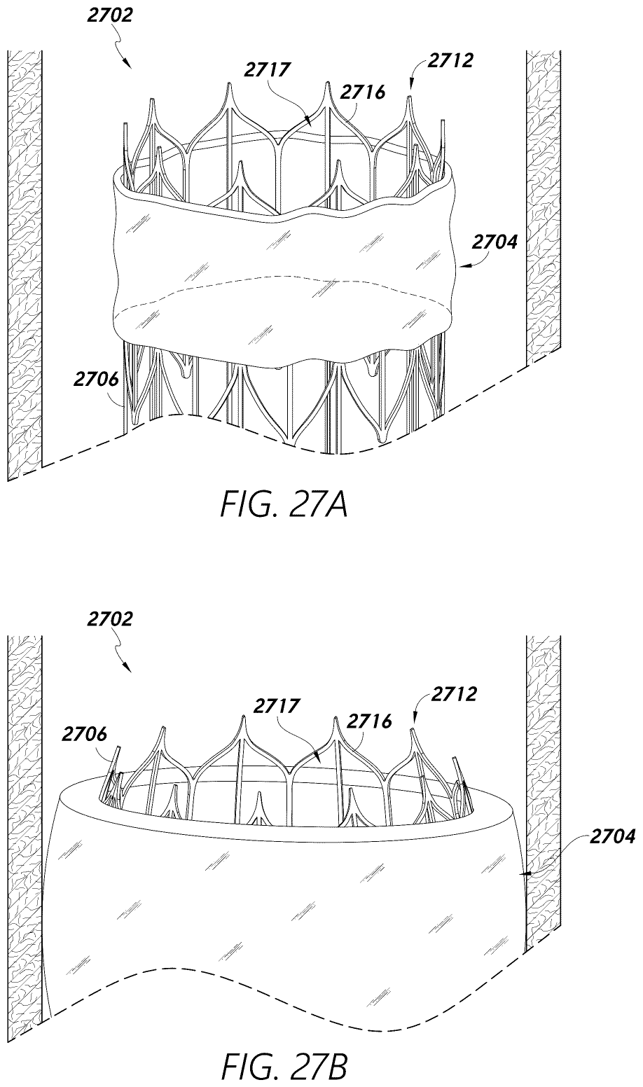

FIGS. 27A-27B illustrate an example implant 2702 comprising an exterior balloon 2704 configured to form a seal between a frame 2706 of the implant 2702 and the surrounding anatomy. FIG. 27A illustrates the implant 2702 with the balloon 2704 in a deflated state. The frame 2706 may have a first expanded form in the deflated state. FIG. 27B illustrates the implant 2702 with the balloon 2704 in an inflated state. The frame 2706 may have a second expanded form in the inflated state in which a width and/or diameter of the frame 2706 exceeds the width and/or diameter in the first expanded form. The exterior balloon 2704 may be configured to inflate to press against irregularities in the patient's anatomy and/or prevent leakage. However, inflation of the balloon 2704 may not necessarily press the balloon 2704 into contact with the surrounding anatomy. For example, gaps may exist between the balloon 2704 and the walls of the blood vessel.

The implant 2702 can be configured for docking one or more prosthetic implants and/or may be configured for other/different uses. In some examples, the implant 2702 may comprise a frame 2706 composed of Nitinol, cobalt chromium, and/or other materials which may be configured to be balloon expanded from the interior and/or exterior of the implant 2702 to a predetermined size and/or to a size of a blood vessel in which the implant may be deployed.

The implant 2702 may comprise an exterior balloon 2704 and can include a scaling skirt disposed at and/or around the balloon 2704. For example, a skirt may form a covering around the balloon 2704 and/or may provide an interface between the frame 2706 and the balloon 2704. The balloon 2704 can be configured to conform to various deviations in the anatomy that otherwise would be difficult to seal against. The balloon 2704 may be further configured to remain with the frame 2706 to maintain the seal.

In some examples, the frame 2706 may be self-expandable. The balloon 2704 may be configured to extend along only a portion of the frame 2706 and/or sealing skirt.

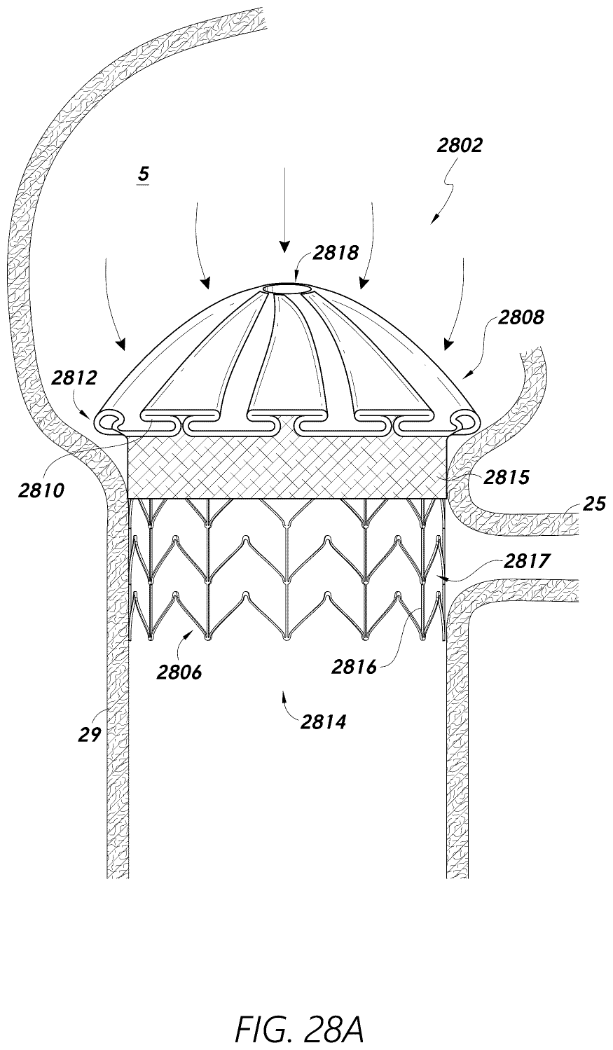

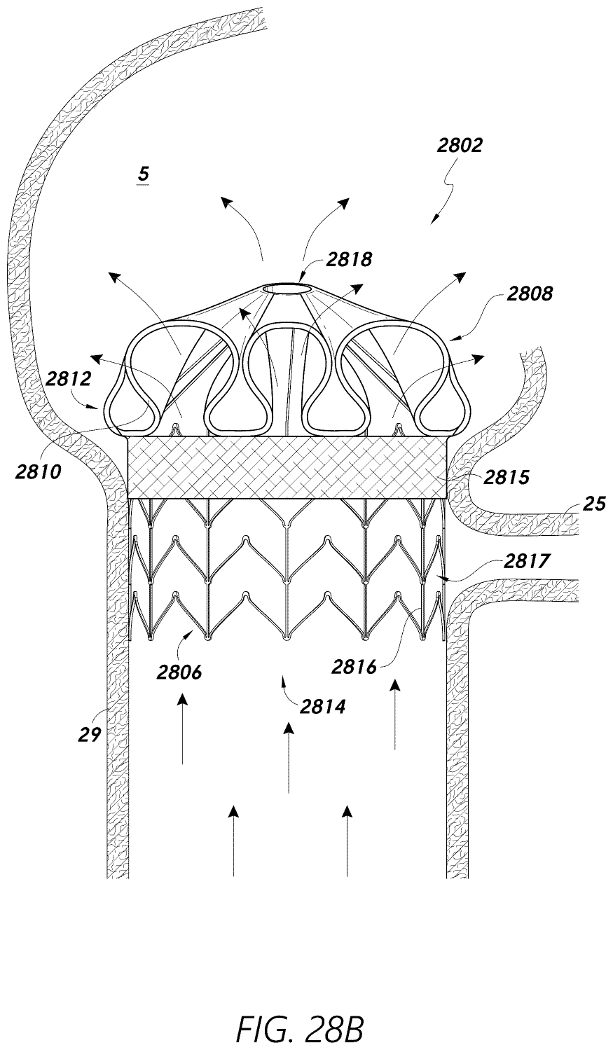

FIGS. 28A-28B illustrate an example implant 2802 configured to mitigate and/or prevent backflow (e.g., from the right atrium 5 into the IVC 29) in accordance with one or more examples. The implant 2802 is shown deployed at an RA/IVC junction, however the implant 2802 may be configured for placement at other anatomical areas. FIG. 28A illustrates that the implant 2802 may be configured to at least partially block downward blood flow (e.g., from the right atrium 5 to the IVC 29). FIG. 28B illustrates that the implant 2802 may additionally or alternatively be configured to at least partially allow upward blood flow (e.g., from the IVC 29 to the right atrium 5).

The implant 2802 may comprise a frame 2806 and/or a cover 2808 extending at least partially along an exterior and/or interior of the frame 2806. The cover 2808 may be disposed at a proximal end 2812 of the frame 2806 (e.g., near and/or at least partially within the right atrium). In some examples, the cover 2808 may be configured to from folds and/or pleats 2810 configured to create one-way flow through the cover 2808. The cover 2808 may be configured to attach and/or couple to the frame 2806 (e.g., to extensions of the frame 2806 at the proximal end 2812 of the frame 2806).

The frame 2806 may be configured expand (e.g., self-expansion and/or balloon expansion). In some examples, the frame 2806 may comprise a network of wires and/or struts 2816 forming one or more cells 2817. The implant 2802 may be configured for implantation at, for example, the RA/IVC junction. However, the implant 2802 may be configured for placement at other anatomical areas. In some examples, the frame 2806 may comprise openings and/or cells 2817 configured to allow blood flow from hepatic veins 25 and/or other branching blood vessels. The implant 2802 may be deployed such that the cells 2817 of the frame 2806 extend over branching blood vessels.

The implant 2802 may comprise a sealing skirt 2815 configured to seal against the surrounding anatomy and/or prevent leakage. The proximal end 2812 end of the frame 2804 may include a cover 2808 over the proximal end 2812 of the frame 2806. The cover 2808 and the sealing skirt 2815 may comprise a common material and/or may extend into each other. However, the cover 2808 and the sealing skirt 2815 may be disconnected and/or separate devices.

The cover 2808 may comprise one or more pleats 2810 (e.g., folds) that may be folded externally to the frame 2806. For example, the cover 2808 may be sized to have a greater surface area than the proximal end 2812 of the frame 2806. In some examples, the cover 2808 may include two or more pleats 2810. For example, pleats 2810 may be disposed around an entire circumference of the proximal end 2812 of the frame 2806. In some examples, the cover 2808 may be attached to extensions of the frame 2806 which may extend generally diagonally from the frame 2806 and/or towards a central point of the frame 2806. The pleats 2810 may comprise folds of the cover 2808 and/or skirt 2815 configured to overlap with other portions of the cover 2808 and/or skirt 2815.

The cover 2808 may include one or more orifices 2818 to allow some backflow through the cover 2808 to preventing clot formation. For example, the cover 2808 may comprise an orifice 2818 at a center point of the cover 2808 (e.g., providing an opening into a lumen of the frame 2806).

As shown in FIG. 28A, the pleats 2810 may be configured to lay flatly and/or form a seal when pressure at the proximal end 2812 of the implant 2802 (e.g., in the right atrium 5) exceeds pressure at a distal end 2814 of the implant 2802 (e.g., in the IVC 29). For example, the cover 2808 may be configured to form a seal during systole to prevent backflow through the pleats 2810. The orifice 2818 may be configured to allow some backflow. As shown in FIG. 28B, the pleats 2810 may be configured to billow and/or open (e.g., stretch and/or extend away from the frame 2806) when pressure at the proximal end 2812 of the implant 2802 (e.g., in the right atrium 5) is below pressure at the distal end 2814 of the implant 2802 (e.g., in the IVC 29). For example, the cover 2808 may be configured to open during diastole to allow forward flow through the pleats 2810.

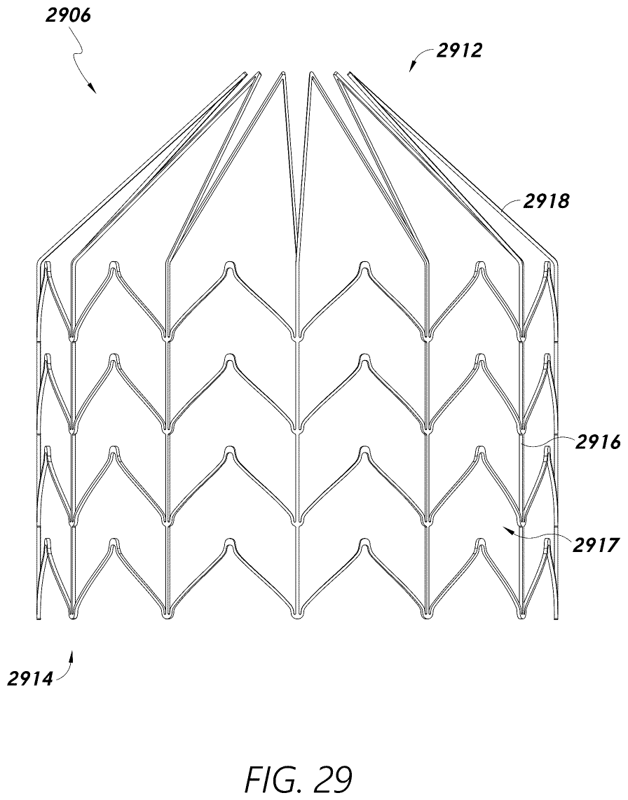

FIG. 29 illustrates an example frame 2906 configured to support and/or provide a skeleton for one or more covers in accordance with one or more examples. In some examples, the frame 2906 may comprise one or more extensions 2918 at a proximal end 2912 of the frame 2906. The extensions 2918 and/or frame 2906 may comprise a network of struts 2916 forming cells 2917. The struts 2916 of the extensions 2918 may have generally triangular forms and/or may be configured to form generally triangle-shaped cells 2917. In some examples, the extensions 2918 may decrease in width towards the proximal end 2912 of the frame 2906 and/or may form a dome-like structure in which the extensions 2918 extend towards each other to create a relatively small opening between end points of the extensions 2918.

The extensions 2918 may be configured to provide a platform and/or base for a cover (e.g., pleated cover). In some examples, struts 2916 of the extensions 2918 may be attached to a cover.

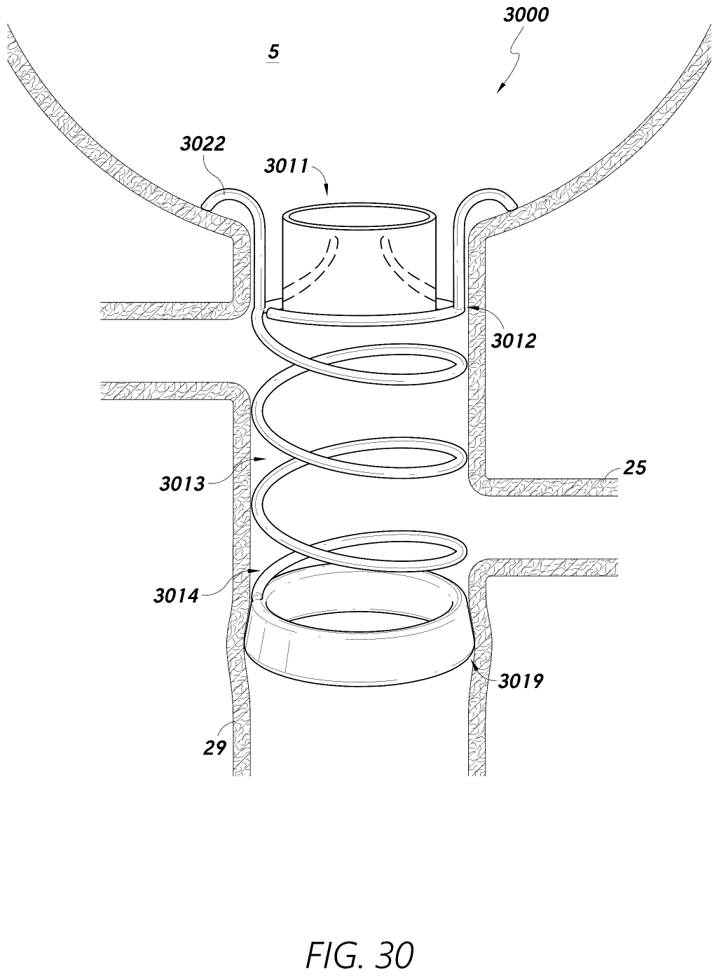

FIG. 30 illustrates an example docking system 3000 for docking one or more prosthetic valves 3011 in accordance with one or more examples. In some examples, the system 3000 may be configured for docking a valve 3011 at or near an IVC/RA junction. The system 3000 may comprise a proximal end 3012 configured to grasp and/or secure the valve 3011. The proximal end 3012 may be configured to be disposed at least partially within the IVC 29 and/or at least partially within the right atrium 5. In some examples, the proximal end 3012 may comprise one or more proximal arms 3022 configured to extend into the right atrium 5 and/or to anchor to surrounding tissue. The one or more proximal arms 3022 may have generally curved forms and/or may be configured to press into and/or against the surrounding tissue.

The system 3000 may comprise a body portion 3013 configured to extend at least partially along the IVC 29 and/or other blood vessel. The body portion 3013 may be configured to extend across inflow junctions of one or more hepatic veins 25 and/or other branching blood vessels. In some examples, the body portion 3013 may comprise a coiled wire, which may form a helical shape. Coils of the body portion 3013 may be configured to form gaps to allow blood flow from the branching blood vessels through the body portion 3013 and/or to prevent blockage of the branching blood vessels. In some examples, the body portion 3013 may extend into the proximal end 3012 and/or a distal end 3014.

The distal end 3014 may comprise a sealing device 3019, which may include a sealing skirt. The sealing device 3019 may be positioned at least partially within the IVC 29 and/or other blood vessel and/or may be disposed inferior to the hepatic veins 25 and/or other branching blood vessels. In some cases, the IVC 29 may have a generally uniform and/or consistent cross-sectional shape inferior to the hepatic veins 25, thereby facilitating creation of a seal. In some examples, the body portion 3013 and/or sealing device 3019 may have an outward bias to press against the blood vessel and/or facilitate anchoring of the system 3000.

The sealing device 3019 may have any suitable size and/or shape. In some examples, the sealing device 3019 may comprise a partial cone shape in which a width and/or diameter of the sealing device 3019 is variable and/or increase towards a proximal or distal side. The increase in width and/or diameter may facilitate anchoring and/or sealing of the sealing device 3019.

In some examples, the proximal end 3012 may comprise a flange and/or other anchoring means. For example, in addition to and/or in place of the proximal arms 3022, the proximal end 3012 may attach to and/or extend into a cylindrical and/or conical flange configured to extend into the right atrium 5.

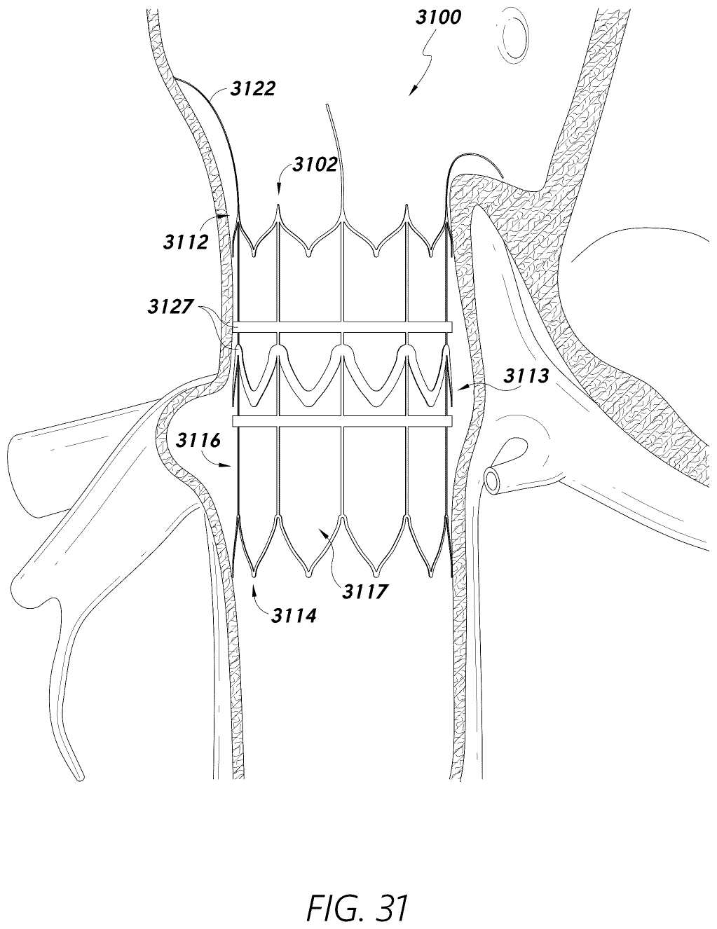

FIG. 31 illustrates an example docking stent 3100 configured for docking one or more prosthetic valves in accordance with one or more examples. The system 3000 may comprise a frame 3102 having a docking portion 3113. The frame 3102 may comprise a network of wires and/or struts 3116 forming one or more cells 3117. The docking portion 3113 may comprise additional struts 3127 which may be configured to extend at least partially across cells 3117 of the frame 3102. In some examples, the additional struts 3127 and/or struts 3116 of the frame 3102 at the docking portion 3113 may be relatively thicker and/or wider relative to other struts 3116 of the frame 3102. The struts 3116 and/or additional struts 3127 of the docking portion 3113 may cause the docking portion 3113 to be relatively stiff and/or rigid relative to other portions of the frame 3102, including a proximal end 3112 and/or distal end 3114 of the frame 3102. In some examples, struts 3116 and/or additional struts 3127 at the docking portion 3113 may have a geometry configured to reinforce and/or stiffens the struts 3116, 3127. Such geometry can include thickening the struts 3116, 3127, adding cross brace struts 3127, and/or other geometries that increase stiffness. This increased stiffness can form an extended landing and/or docking zone for one or more prosthetic valves.

In some patients, an IVC/RA junction may be relatively short. In such cases, the docking portion 3113 may create an extended landing zone to increase an area of the system 3000 for receiving the one or more prosthetic valves. Moreover, the docking portion 3113 may reduce the possibility of the one or more prosthetic valves shifting.

The frame 3102 and/or docking portion 3113 may comprise an inner lumen configured to receive a prosthetic valve. In some examples, the frame 3102 and/or docking portion 3113 may comprise one or more inner walls and/or arms configured to grasp and/or receive a prosthetic valve.

The frame 3102 and/or docking portion 3113 may be composed of any suitable material, which can include Nitinol. In some examples, the system 3000 may comprise a sealing skirt sewn and/or attached to the frame 3102.

In some examples, the proximal end 3112 may comprise one or more proximal arms 3122 configured to extend into the right atrium and/or to anchor to surrounding tissue. The one or more proximal arms 3122 may have generally curved forms and/or may press into and/or against the surrounding tissue.

The RA/IVC junction may provide little anatomical space for anchoring various docking systems. Moreover, the RA/IVC junction may have irregularity and/or variability from patient to patient, which may create uncertainty and/or instability in anchoring at the RA/IVC junction. Examples described herein may comprise various features to facilitate anchoring at such anatomies.

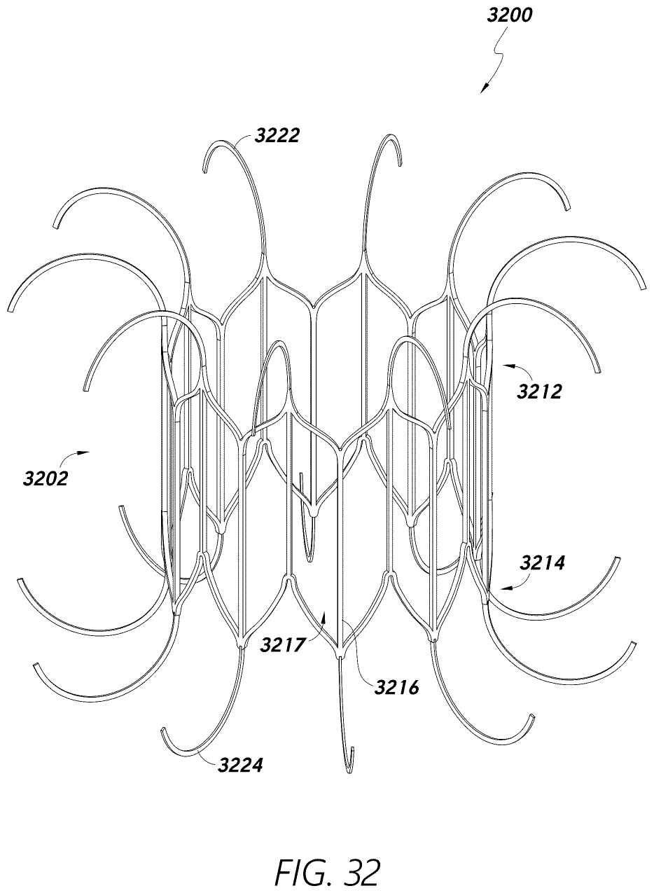

FIG. 32 illustrates an example docking stent 3200 comprising one or more proximal arms 3222 and/or distal arms 3224 and configured for docking one or more prosthetic valves in accordance with one or more examples. The system 3000 may comprise a frame 3202 and/or midsection configured for docking one or more prosthetic valves. The frame 3202 may comprise a network of wires and/or struts 3216 forming one or more cells 3217 (e.g., openings).

The frame 3202 and/or arms may be composed of any suitable material, which can include Nitinol. In some examples, the stent 3000 may comprise a scaling skirt sewn and/or attached to the frame 3202. For example, a cylindrical skirt may extend along an inner and/or outer surface of the frame 3202 (e.g., between the proximal arms 3222 and the distal arms 3224).

The frame 3202 may comprise a proximal end 3212 and/or a distal end 3214. In some examples, the proximal end 3212 may comprise one or more proximal arms 3222 configured to extend into the right atrium and/or to anchor to surrounding tissue and/or the distal end 3214 may comprise one or more distal arms 3224 configured to extend along the IVC and/or extend into various branching blood vessels (e.g., hepatic veins). The one or more proximal arms 3222 and/or distal arms 3224 may have generally curved forms and/or may be configured to press into and/or against the surrounding tissue.

In some examples, the proximal arms 3222 and/or distal arms 3224 may extend from struts 3216 of the frame 3202. The proximal arms 3222 and/or distal arms 3224 may be shape-set to a curled and/or curved form such that the proximal arms 3222 may extend towards the distal end 3214 and/or the distal arms 3224 may extend towards the proximal end 3212. The proximal arms 3222 and/or distal arms 3224 may be configured to grip the surrounding anatomy (e.g., the hepatic veins). The proximal arms 3222 and distal arms 3224 may be configured to work together to hold the stent 3200 in place and/or prevent migration of the stent 3200.

The stent 3200 may comprise a sealing skirt. The sealing skirt may have a cylindrical form and/or may at least partially enclose a cylindrical area of the frame 3202 (e.g., at a midsection of the frame 3202). In some examples, the sealing skirt may extend along an exterior surface of the frame 3202. The sealing skirt may be disposed between the proximal end 3212 and the distal end 3214.

The stent 3200 may comprise any number of proximal arms 3222 and/or any number of distal arms 3224. In some examples, the stent 3200 may comprise three or more proximal arms 3222 and/or three or more distal arms 3224. The stent 3200 may comprise an outer frame and an inner frame. In some examples, one or more of the proximal arms 3222 and/or distal arms 3224 may extend from the inner frame and/or from the outer frame. The inner frame and/or arms extending from the inner frame may be configured to receive and/or dock a prosthetic valve.

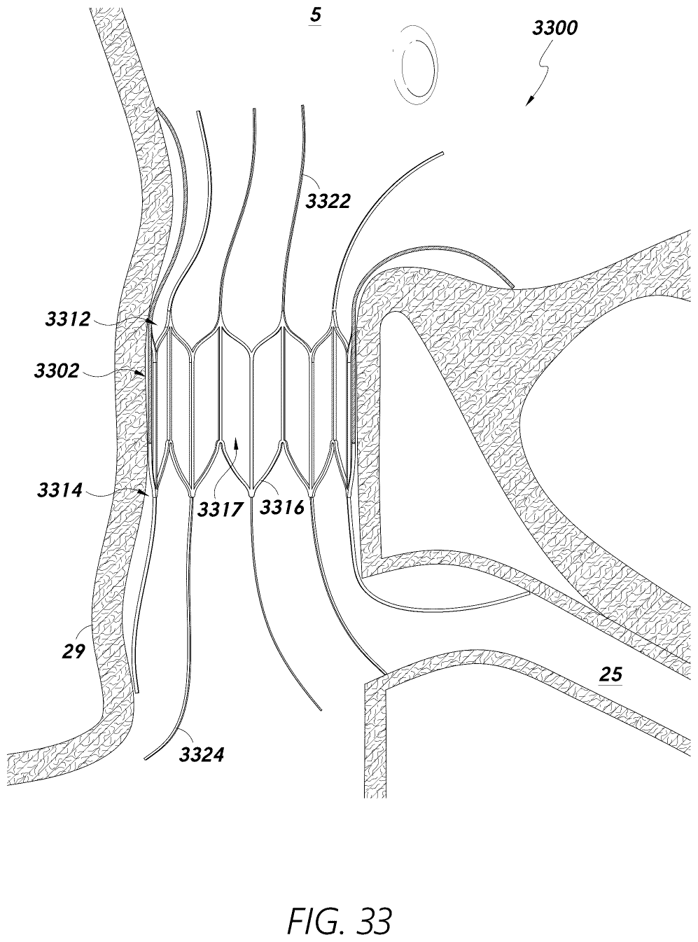

FIG. 33 illustrates an example docking stent 3300 comprising one or more proximal arms 3322 and/or distal arms 3324 deployed at an RA/IVC junction in accordance with one or more examples. The system 3000 may comprise a frame 3302 and/or midsection configured for docking one or more prosthetic valves. The frame 3302 may comprise a network of wires and/or struts 3316 forming one or more cells 3317.

The frame 3302 and/or arms may be composed of any suitable material, which can include Nitinol. In some examples, the stent 3000 may comprise a sealing skirt sewn and/or attached to the frame 3302.

The frame 3302 may comprise a proximal end 3312 and/or a distal end 3314. In some examples, the proximal end 3312 may comprise one or more proximal arms 3322 configured to extend into the right atrium 5 and/or to anchor to surrounding tissue and/or the distal end 3314 may comprise one or more distal arms 3324 configured to extend along the IVC 29 and/or extend into various branching blood vessels (e.g., hepatic veins 25). The one or more proximal arms 3322 and/or distal arms 3324 may have generally curved forms and/or may press into and/or against the surrounding tissue.

In some examples, the proximal arms 3322 and/or distal arms 3324 may extend from struts 3316 of the frame 3302. The proximal arms 3322 and/or distal arms 3324 may be shape set to a curled and/or curved form such that the proximal arms 3322 may extend towards the distal end 3314 and/or the distal arms 3324 may extend towards the proximal end 3312. The proximal arms 3322 and/or distal arms 3324 may be configured to grip the surrounding anatomy (e.g., the hepatic veins). The proximal arms 3322 and distal arms 3324 may be configured to work together to hold the stent 3300 in place and/or prevent migration of the stent 3300.

The stent 3300 may comprise a sealing skirt. The sealing skirt may have a cylindrical form and/or may at least partially enclose a cylindrical area of the frame 3302 (e.g., at a midsection of the frame 3302). In some examples, the sealing skirt may extend along an exterior surface of the frame 3302. The sealing skirt may be disposed between the proximal end 3312 and the distal end 3314.

The stent 3300 may comprise any number of proximal arms 3322 and/or any number of distal arms 3324. In some examples, the stent 3300 may comprise three or more proximal arms 3322 and/or three or more distal arms 3324. The stent 3300 may comprise an outer frame and an inner frame. In some examples, one or more of the proximal arms 3322 and/or distal arms 3324 may extend from the inner frame and/or from the outer frame. The inner frame and/or arms extending from the inner frame may be configured to receive and/or dock a prosthetic valve.

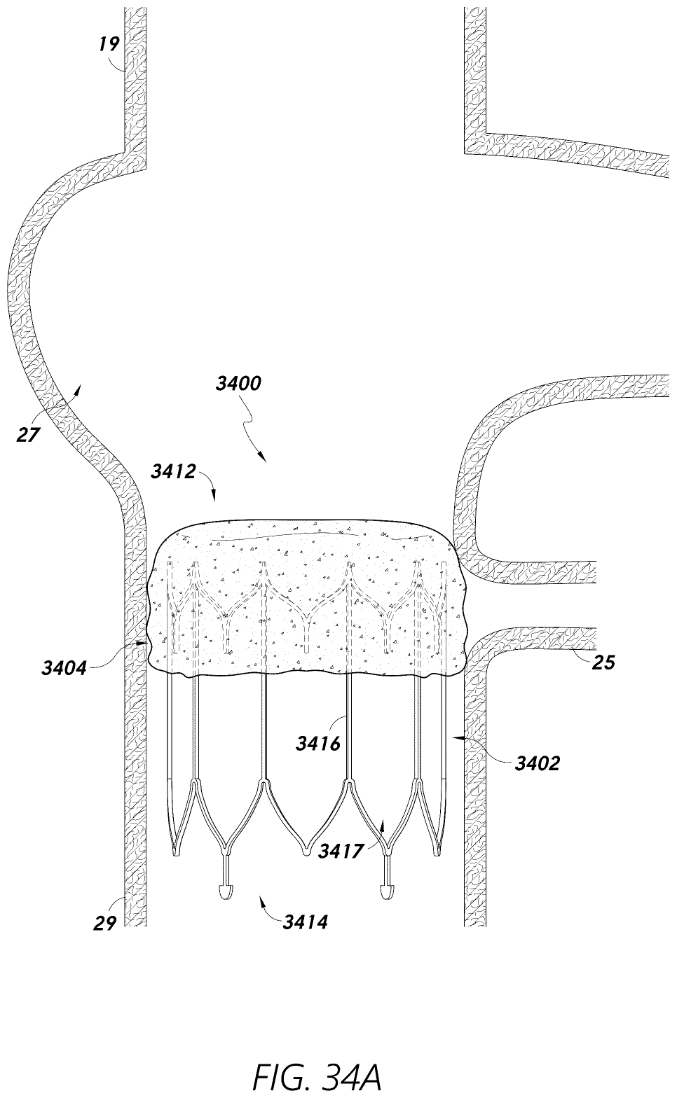

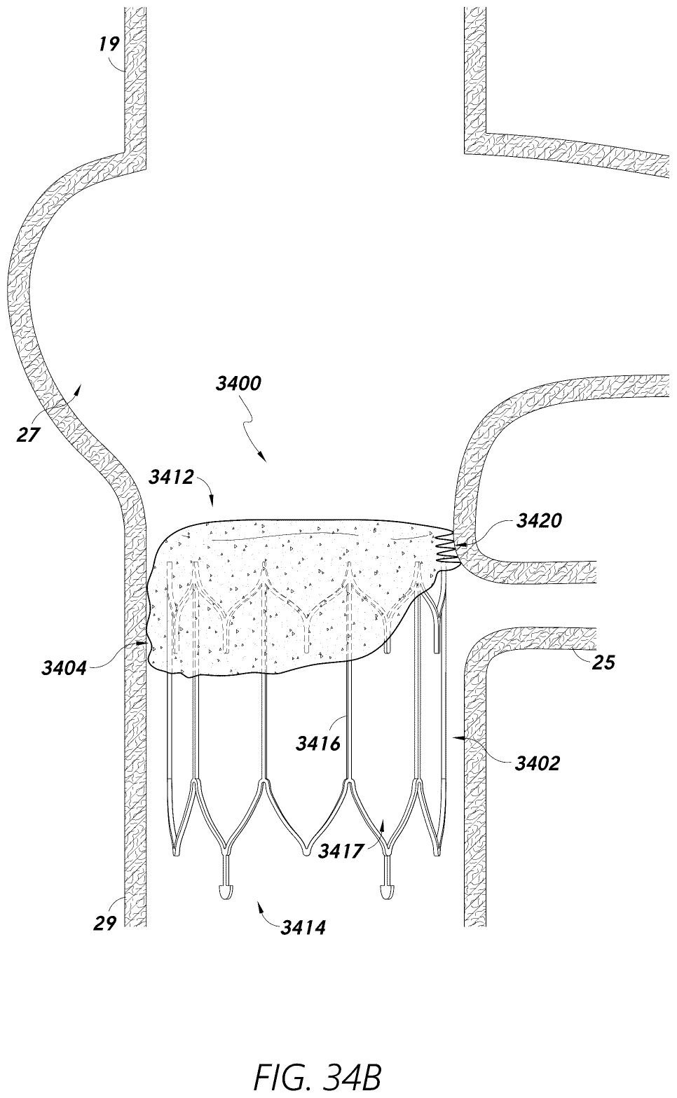

FIGS. 34A and 34B illustrate an example docking stent 3400 comprising an adjustable sealing skirt 3404 in accordance with one or more examples. FIG. 34A illustrates the stent 3400 disposed within an IVC 29 in an unadjusted form. FIG. 34B illustrates the stent 3400 disposed within the IVC 29 in an adjusted form in which the sealing skirt 3404 is raised at least partially on at least one side to form a compressed portion 3420 of the skirt 3404. In some examples, the stent 3400 may be configured for sealing below a posterior pouch 27 of a heart. For example, the stent 3400 may be configured for placement at least partially within an IVC 29 and/or may be configured to extend across one or more hepatic veins 25 and/or branching blood vessels. The stent 3400 may comprise various means for adjusting the skirt 3404 to prevent and/or mitigate blockage of the branching blood vessels.

The skirt 3404 may be configured to be adjustable along a length of the frame 3402 and/or may be capable of being raised and/or lowered to a more proximal position (e.g., closer to a proximal end 3412 and/or further from a distal end 3414) relative to other portions of the skirt 3404. In this way, the stent 3400 may be anchored at a distance away from the posterior pouch 27 without fully blocking one or more branching blood vessels.

The skirt 3404 may be adjusted by any suitable means. For example, one or more sutures and/or lines may be attached to and/or may extend at least partially through the skirt 3404 to attach to and/or form a loop around the skirt 3404. At least a portion of the skirt 3404 can be raised by pulling on one or more ends of the attached suture and/or line to at least partially withdraw the suture and/or line and/or to cause pushing and/or pulling force at at least a portion of the skirt 3404.

In some examples, the stent 3400 may comprise a frame 3402 comprising a network of struts 3416 forming one or more cells 3417. The skirt 3404 may extend along only a portion of the frame 3402 (e.g., at or near the proximal end 3412 of the stent 3400). In some cases, a hepatic vein 25 and/or branching blood vessel may be approximately 10 mm or less (e.g., 4 mm) below the RA/IVC junction. In some examples, the skirt 3404 may have a length greater than 4 mm and/or 10 mm to facilitate proper sealing of the stent 3400. While the stent 3400 may be deployed higher in the IVC 25 (e.g., extending into the RA 5) to avoid extending across the hepatic veins 25, doing so may create undesired and/or unnecessary leak paths around and/or through the stent 3400. The adjustable skirt 3404 may allow for the stent 3400 to be deployed lower (e.g., fully within the IVC 29) without fully blocking one or more branching blood vessels by placing cells 3417 (e.g., openings) across the branching blood vessels.

In some cases, gaps may form between stents disposed within an IVC 29 and the vessel walls of the IVC 29. It may be advantageous to fill such gaps to create a proper seal and/or anchor.

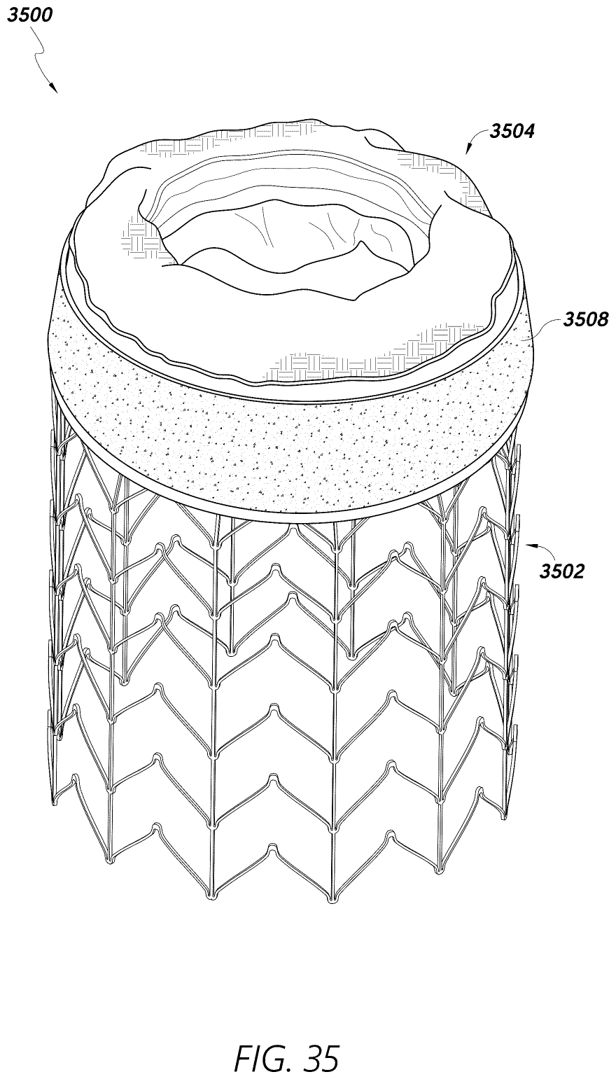

FIG. 35 illustrates a docking stent 3500 comprising a sealing portion 3508 (e.g., band, strip, ring) configured to close gaps between the stent 3500 and the surrounding tissue, in accordance with one or more examples. The sealing portion 3508 may be disposed at and/or around an external surface of a sealing skirt 3504 extending at least partially along a frame 3502 of the stent 3500. In some examples, the sealing portion 3508 may be at least partially compressible and/or expandable. The sealing portion 3508 may be attached to and/or may extend from the sealing skirt 3504 of the stent 3500.

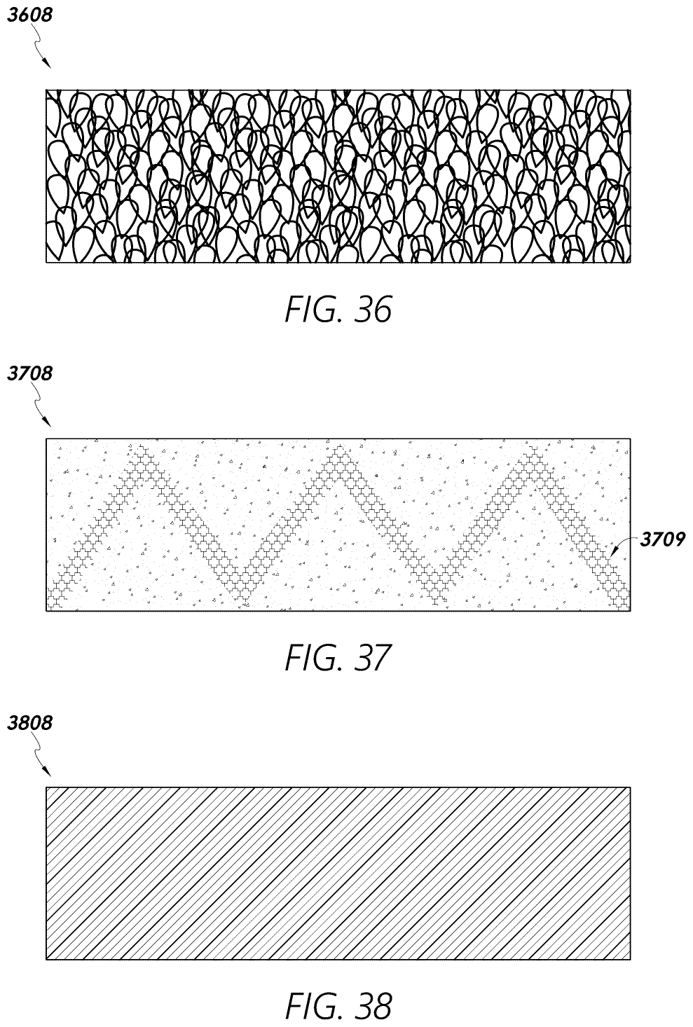

In some examples, the sealing skirt 3504 may comprise one or more generally flat and/or woven textiles and/or materials. The sealing portion 3508 may have any suitable structure and/or may comprise any suitable materials (see, e.g., FIGS. 36-38). For example, the sealing portion 3508 may comprise a looped material (e.g., terrycloth-like) and/or can fill gaps around a circumferential area of the sealing skirt 3504 and/or frame 3502.

FIG. 36 illustrates an example sealing portion 3608 comprising terry-cloth and/or other material comprising loops and/or fibers, in accordance with one or more examples. The sealing portion 3608 is shown from a side view and/or may have a generally cylindrical form to at least partially and/or fully surround a scaling skirt and/or frame of a stent. The material(s) of the scaling portion 3608 can support clotting of blood in the scaling area to close off the sealing portion 3608 and/or stent, thereby preventing leaks.

FIG. 37 illustrates an example scaling portion 3708 comprising tufts 3709 added to the sealing portion 3708 material using multifilament thread or sutures, in accordance with one or more examples. The tufts 3709 may be knotted, trimmed, and/or separated to create fuzz and/or filaments that can close gaps. While the tufts 3709 are shown in a zig-zag pattern, other patterns may be used. The scaling portion 3708 is shown from a side view and/or may have a generally cylindrical form to at least partially and/or fully surround a sealing skirt and/or frame of a stent. The material(s) of the sealing portion 3708 can support clotting of blood in the sealing area to close off the scaling portion 3708 and/or stent, thereby preventing leaks.

FIG. 38 illustrates an example scaling portion 3808 comprising a polymer coating configured to react to blood to expand and/or fill gaps between a stent and surrounding tissue, in accordance with one or more examples. The sealing portion 3808 is shown from a side view and/or may have a generally cylindrical form to at least partially and/or fully surround a sealing skirt and/or frame of a stent.

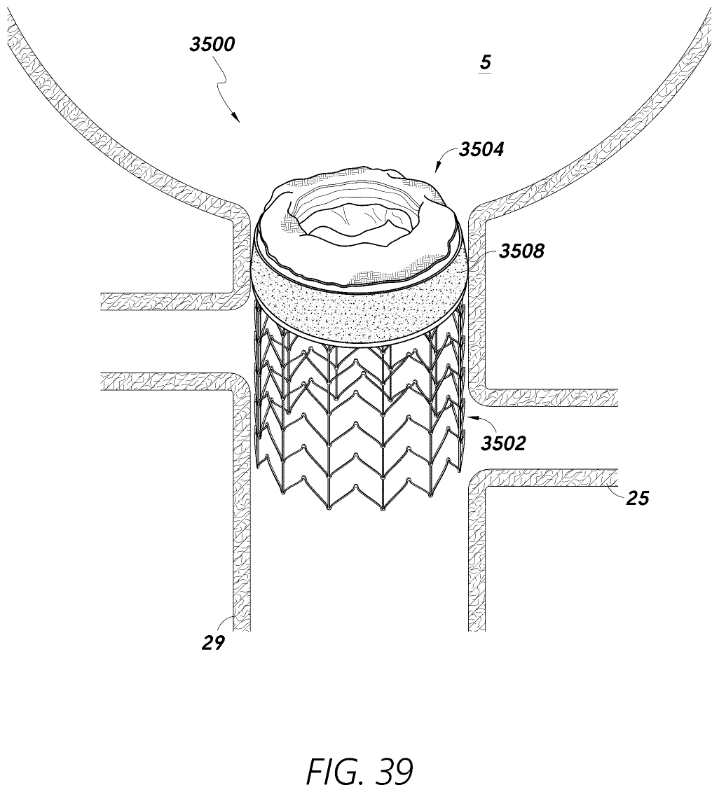

FIG. 39 illustrates a docking stent 3900 comprising a scaling portion 3908 configured to close gaps between the stent 3900 and the surrounding tissue, in accordance with one or more examples. The sealing portion 3908 may be disposed at an external surface of a sealing skirt 3904 extending at least partially along a frame 3902 of the stent 3900. In some examples, the scaling portion 3908 may be at least partially compressible and/or expandable. The sealing portion 3908 may be attached to and/or may extend from the scaling skirt 3904 of the stent 3900.

In some examples, the sealing skirt 3904 may comprise one or more generally flat and/or woven textiles and/or materials. The scaling portion 3908 may have any suitable structure and/or may comprise any suitable materials (see, e.g., FIGS. 36-38). For example, the scaling portion 3908 may comprise a looped material (e.g., terrycloth-like) and/or can fill gaps around a circumferential area of the sealing skirt 3904 and/or frame 3902.

The stent 3900 may be configured for placement at least partially within an IVC 29 of a heart. The sealing portion 3908 may be configured for placement between the RA 5 and/or a hepatic vein 25 and/or other branching blood vessel.

### Additional Examples

The treatment techniques, methods, steps, etc. described or suggested herein or in references incorporated herein can be performed on a living animal or on a non-living simulation, such as on a cadaver, cadaver heart, anthropomorphic ghost, simulator (e.g., with the body parts, tissue, etc. being simulated), etc.

Depending on the example, certain acts, events, or functions of any of the processes or algorithms described herein can be performed in a different sequence, may be added, merged, or left out altogether. Thus, in certain examples, not all described acts or events are necessary for the practice of the processes.

Example 1: A cardiac implant device comprising: a frame configured for placement at least partially within a blood vessel of a heart; and a skirt configured to improve a seal of the frame within the blood vessel.

Example 2: The cardiac implant device of any example herein, in particular example 1, wherein the skirt at least partially encloses an outer surface of the frame.

Example 3: The cardiac implant device of any example herein, in particular example 1, wherein the skirt at least partially encloses an inner surface of the frame.

Example 4: The cardiac implant device of any example herein, in particular example 1, wherein the skirt extends from a distal end of the frame to midsection of the frame.

Example 5: The cardiac implant device of any example herein, in particular example 1, wherein the skirt comprises one or more fabrics.

Example 6: The cardiac implant device of any example herein, in particular example 1, wherein the skirt comprises a relatively thin portion and a relatively thick portion.

Example 7: The cardiac implant device of any example herein, in particular example 6, wherein the relatively thick portion has a zigzag form.

Example 8: The cardiac implant device of any example herein, in particular example 7, wherein the relatively thick portion extends around a circumference of the skirt.

Example 9: The cardiac implant device of any example herein, in particular example 6, wherein the relatively thick portion forms an elongate column extending longitudinally along the frame.

Example 10: The cardiac implant device of any example herein, in particular example 9, wherein the skirt comprises multiple relatively thin portions and multiple relatively thick portions, and wherein the relatively thin portions and the relatively thick portions are positioned in an alternating manner.

Example 11: The cardiac implant device of any example herein, in particular example 10, wherein the relatively thin portions extend gradually into the relatively thick portions.

Example 12: The cardiac implant device of any example herein, in particular example 6, wherein the relatively thick portion comprises an elongate form and extends laterally relative to the frame.

Example 13: The cardiac implant device of any example herein, in particular example 12, wherein the skirt comprises multiple relatively thick portions.

Example 14: The cardiac implant device of any example herein, in particular example 13, wherein the multiple relatively thick portions are disposed in multiple rows and in a staggered manner relative to adjacent rows.

Example 15: The cardiac implant device of any example herein, in particular example 6, wherein the relatively thick portion has a half dome shape.

Example 16: The cardiac implant device of any example herein, in particular example 1, further comprising one or more flaps coupled to an exterior surface of the skirt.

Example 17: The cardiac implant device of any example herein, in particular example 16, wherein the one or more flaps have triangular shapes.

Example 18: The cardiac implant device of any example herein, in particular example 16, wherein the one or more flaps comprise base portions coupled to the skirt and further comprise free ends.

Example 19: The cardiac implant device of any example herein, in particular example 16, wherein the one or more flaps are arranged in series.

Example 20: The cardiac implant device of any example herein, in particular example 19, wherein the one or more flaps form a helical pattern around the skirt.

Example 21: The cardiac implant device of any example herein, in particular example 1, wherein the skirt comprises a base coupled to a distal end of the frame and further comprises a free end.

Example 22: The cardiac implant device of any example herein, in particular example 21, wherein the skirt has a tubular form.

Example 23: The cardiac implant device of any example herein, in particular example 1, wherein the frame comprises a lateral extension configured for placement within a branching blood vessel.

Example 24: The cardiac implant device of any example herein, in particular example 23, wherein the lateral extension has a tubular form.

Example 25: The cardiac implant device of any example herein, in particular example 1, wherein the skirt has a ring shape.

Example 26: The cardiac implant device of any example herein, in particular example 25, wherein the skirt has a non-circular shape and extends further from the frame in a first direction than in a second direction.

Example 27: The cardiac implant device of any example herein, in particular example 1, wherein the skirt comprises a rigid portion and a flexible portion.

Example 28: The cardiac implant device of any example herein, in particular example 27, wherein the flexible portion at least partially encloses the rigid portion.

Example 29: The cardiac implant device of any example herein, in particular example 27, wherein the rigid portion is expandable.

Example 30: The cardiac implant device of any example herein, in particular example 27, wherein the rigid portion and the flexible portion comprise flared ends.

Example 31: The cardiac implant device of any example herein, in particular example 1, wherein the skirt comprises one or more apertures disposed at a distal end of the skirt.

Example 32: The cardiac implant device of any example herein, in particular example 1, wherein the frame comprises an inner arm extending into an inner lumen of the frame.

Example 33: The cardiac implant device of any example herein, in particular example 32, wherein the skirt loops at least partially around the inner arm.

Example 34: The cardiac implant device of any example herein, in particular example 33, wherein the skirt is at least partially stretchy.

Example 35: The cardiac implant device of any example herein, in particular example 1, wherein the frame comprises one or more proximally extending arms.

Example 36: A valve docking device comprising: a frame configured for placement at least partially within a blood vessel of a heart; and a skirt configured to improve a seal of the frame within the blood vessel.

Example 37: The valve docking device of any example herein, in particular example 36, wherein the skirt at least partially encloses an outer surface of the frame.

Example 38: The valve docking device of any example herein, in particular example 36, wherein the skirt at least partially encloses an inner surface of the frame.

Example 39: The valve docking device of any example herein, in particular example 36, wherein the skirt extends from a distal end of the frame to midsection of the frame.

Example 40: The valve docking device of any example herein, in particular example 36, wherein the skirt comprises one or more fabrics.

Example 41: The valve docking device of any example herein, in particular example 36, wherein the skirt comprises a relatively thin portion and a relatively thick portion.

Example 42: The valve docking device of any example herein, in particular example 41, wherein the relatively thick portion has a zigzag form.

Example 43: The valve docking device of any example herein, in particular example 42, wherein the relatively thick portion extends around a circumference of the skirt.

Example 44: The valve docking device of any example herein, in particular example 41, wherein the relatively thick portion forms an elongate column extending longitudinally along the frame.

Example 45: The valve docking device of any example herein, in particular example 44, wherein the skirt comprises multiple relatively thin portions and multiple relatively thick portions, and wherein the relatively thin portions and the relatively thick portions are positioned in an alternating manner.

Example 46: The valve docking device of any example herein, in particular example 45, wherein the relatively thin portions extend gradually into the relatively thick portions.

Example 47: The valve docking device of any example herein, in particular example 41, wherein the relatively thick portion comprises an elongate form and extends laterally relative to the frame.

Example 48: The valve docking device of any example herein, in particular example 47, wherein the skirt comprises multiple relatively thick portions.

Example 49: The valve docking device of any example herein, in particular example 48, wherein the multiple relatively thick portions are disposed in multiple rows and in a staggered manner relative to adjacent rows.

Example 50: The valve docking device of any example herein, in particular example 41, wherein the relatively thick portion has a half dome shape.

Example 51: The valve docking device of any example herein, in particular example 36, further comprising one or more flaps coupled to an exterior surface of the skirt.

Example 52: The valve docking device of any example herein, in particular example 51, wherein the one or more flaps have triangular shapes.

Example 53: The valve docking device of any example herein, in particular example 51, wherein the one or more flaps comprise base portions coupled to the skirt and further comprise free ends.

Example 54: The valve docking device of any example herein, in particular example 51, wherein the one or more flaps are arranged in series.

Example 55: The valve docking device of any example herein, in particular example 54, wherein the one or more flaps form a helical pattern around the skirt.

Example 56: The valve docking device of any example herein, in particular example 36, wherein the skirt comprises a base coupled to a distal end of the frame and further comprises a free end.

Example 57: The valve docking device of any example herein, in particular example 56, wherein the skirt has a tubular form.

Example 58: The valve docking device of any example herein, in particular example 36, wherein the frame comprises a lateral extension configured for placement within a branching blood vessel.

Example 59: The valve docking device of any example herein, in particular example 58, wherein the lateral extension has a tubular form.

Example 60: The valve docking device of any example herein, in particular example 36, wherein the skirt has a ring shape.

Example 61: The valve docking device of any example herein, in particular example 60, wherein the skirt has a non-circular shape and extends further from the frame in a first direction than in a second direction.

Example 62: The valve docking device of any example herein, in particular example 36, wherein the skirt comprises a rigid portion and a flexible portion.

Example 63: The valve docking device of any example herein, in particular example 62, wherein the flexible portion at least partially encloses the rigid portion.

Example 64: The valve docking device of any example herein, in particular example 62, wherein the rigid portion is expandable.

Example 65: The valve docking device of any example herein, in particular example 62, wherein the rigid portion and the flexible portion comprise flared ends.

Example 66: The valve docking device of any example herein, in particular example 36, wherein the skirt comprises one or more apertures disposed at a distal end of the skirt.

Example 67: The valve docking device of any example herein, in particular example 36, wherein the frame comprises an inner arm extending into an inner lumen of the frame.

Example 68: The valve docking device of any example herein, in particular example 67, wherein the skirt loops at least partially around the inner arm.

Example 69: The valve docking device of any example herein, in particular example 68, wherein the skirt is at least partially stretchy.

Example 70: The valve docking device of any example herein, in particular example 36, wherein the frame comprises one or more proximally extending arms.

Example 71: A cardiac implant device and/or the valve docking device any example herein, in particular example 36, wherein the skirt is inflatable and configured to press the frame towards walls of the blood vessel.

Example 72: A cardiac implant device and/or the valve docking device any example herein, in particular example 36, wherein the frame comprises extensions at a proximal end of the frame and the skirt forms flaps over the extensions.

Example 73: A cardiac implant device and/or the valve docking device any example herein, in particular example 36, wherein the frame comprises a coiled body portion.

Example 74: A cardiac implant device and/or the valve docking device any example herein, in particular example 36, wherein the frame comprises a network of struts, and wherein a first set of struts at a midsection of the frame are thicker than other struts of the network of struts.

Example 75: A cardiac implant device and/or the valve docking device any example herein, in particular example 36, wherein the frame comprises one or more proximal arms extending from a proximal end of the frame and one or more distal arms extending from a distal end of the frame, and wherein the one or more proximal arms and/or one or more distal arms are shape-set in a curved form.

Example 76: A cardiac implant device and/or the valve docking device any example herein, in particular example 36, wherein the skirt is adjustable along a length of the frame to mitigate blockage of one or more branching blood vessels.

Example 77: A cardiac implant device and/or the valve docking device any example herein, in particular example 36, further comprising a sealing band disposed around the skirt.

Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.).

Depending on the example, certain acts, events, or functions of any of the processes or algorithms described herein can be performed in a different sequence, may be added, merged, or left out altogether. Thus, in certain examples, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain examples include, while other examples do not include, certain features, elements and/or steps. Thus, such conditional language is not generally intended to imply that features, elements and/or steps are in any way required for one or more examples or that one or more examples necessarily include logic for deciding, with or without author input or prompting, whether these features, elements and/or steps are included or are to be performed in any particular example. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain examples require at least one of X, at least one of Y and at least one of Z to each be present.

It should be appreciated that in the above description of examples, various features are sometimes grouped together in a single example, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular example herein can be applied to or used with any other example(s). Further, no component, feature, step, or group of components, features, or steps are necessary or indispensable for each example. Thus, it is intended that the scope of the inventions herein disclosed and claimed below should not be limited by the particular examples described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example examples belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality.

What is claimed is:

1. A cardiac implant device comprising:
a frame configured for placement at least partially within a blood vessel of a heart;
a skirt configured to at least partially enclose the frame; and
two or more arms extending from an end of the frame.

2. The cardiac implant device of claim 1, wherein the two or more arms are at least partially curved.

3. The cardiac implant device of claim 2, wherein the two or more arms have a wavy form.

4. The cardiac implant device of claim 2 or claim 3, wherein the two or more arms form a bulb.

5. The cardiac implant device of claim 2, wherein the two or more arms are configured to straighten in response to blood pressure through the frame.

6. The cardiac implant device of claim 1, wherein the skirt is further configured to at least partially enclose the two or more arms.

7. A cardiac implant device comprising:
a frame configured for placement at least partially within a blood vessel of a heart; and
a skirt configured to improve a seal of the frame within the blood vessel,
wherein the skirt comprises a relatively thin portion and a relatively thick portion.

8. The cardiac implant device of claim 7, wherein the relatively thick portion has a zigzag form.

9. The cardiac implant device of claim 8, wherein the relatively thick portion extends around a circumference of the skirt.

10. The cardiac implant device of claim 7, wherein the relatively thick portion forms an elongate column extending longitudinally along the frame.

11. The cardiac implant device of claim 10, wherein the skirt comprises multiple relatively thin portions and multiple relatively thick portions, and wherein the relatively thin portions and the relatively thick portions are positioned in an alternating manner.

12. The cardiac implant device of claim 7, wherein the frame comprises a lateral extension configured for placement within a branching blood vessel.

13. The cardiac implant device of claim 7, wherein the frame comprises an inner arm extending into an inner lumen of the frame.

14. The cardiac implant device of claim 7, wherein the skirt is inflatable.

15. The cardiac implant device of claim 7, wherein the frame comprises extensions at a proximal end of the frame and the skirt forms flaps over the extensions.

16. The cardiac implant device of claim 7, wherein the frame comprises a coiled body portion.

17. The cardiac implant device of claim 7, wherein the frame comprises one or more proximal arms extending from a proximal end of the frame and one or more distal arms extending from a distal end of the frame.

18. The cardiac implant device of claim 17, wherein the one or more proximal arms and/or one or more distal arms are shape-set in a curved form.

19. A cardiac implant device comprising:
a frame configured for placement at least partially within a blood vessel of a heart;
a skirt configured to improve a seal of the frame within the blood vessel, and
one or more flaps coupled to an exterior surface of the skirt, the one or more flaps arranged in series forming a helical pattern around the skirt.

20. The cardiac implant device of claim 19, wherein the frame comprises extensions at a proximal end of the frame.
